(19) United States
(12) Patent Application Publication
(10) Pub. No.: US 2006/0058361 A1
(43) Pub. Date: Mar. 16, 2006

(54) Therapeutic diphenyl ether ligands

(75) Inventors: Anton J. Fliri, Stonington, CT (US); Christopher J. O'Donnell, Mystic, CT (US); Michelle Claffey, Stonington, CT (US); Randall J. Gallaschun, Lebanon, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/223,516

(22) Filed: Sep. 9, 2005

Related U.S. Application Data
(60) Provisional application No. 60/608,994, filed on Sep. 10, 2004.
(60) Provisional application No. 60/699,175, filed on Jul. 14, 2005.

(51) Int. Cl. A61K 31/428; A61K 31/423; A61K 31/421; A61K 31/4184; A61K 31/277; A61K 31/137

(52) U.S. Cl. 514/367; 514/374; 514/375; 514/394; 514/522; 514/534; 514/649

(57) ABSTRACT

This invention is directed to compounds of formula Ia, Ib or Ic and to pharmaceutical compositions thereof: 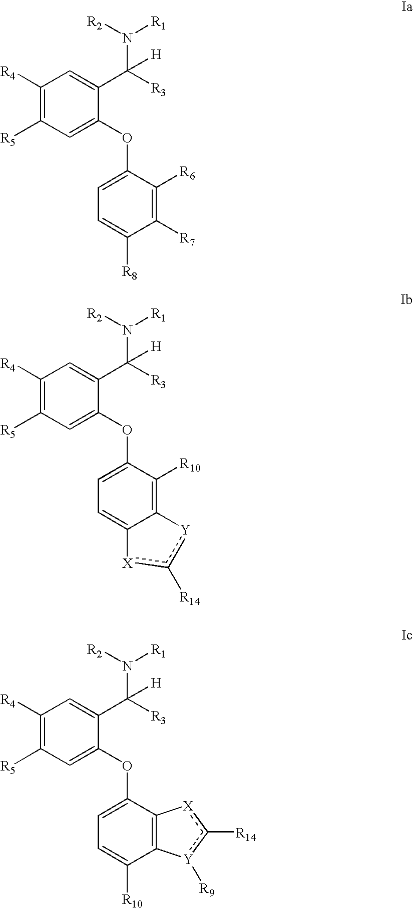 or a prodrug thereof and a pharmaceutically acceptable carrier, wherein the R groups are defined in the specification; and, in which the dashed line represents an optional double bond. The invention is also directed to methods of treating, diagnosing, and preventing disorders of the central nervous system that are associated with 5HT receptors, including obesity, attention deficit disorder, migraine, depression, epilepsy, anxiety, Alzheimer's disease, withdrawal from drug abuse, pain, schizophrenia, stress-related disorders, panic disorder, sleep disorders, phobias, obsessive compulsive disorder, post-traumatic-stress syndrome, immune system depression, stress-induced gastrointestinal dysfunction, stress-induced cardiovascular dysfunction, and sexual dysfunction.

### FIELD OF THE INVENTION

The present invention provides diphenyl ether derivatives, and, more specifically, provides compounds of Formula I described hereinbelow. These compounds are serotonin (“5HT”) receptor ligands and are useful for treating diseases wherein modulation of serotonin activity is desired.

### BACKGROUND OF THE INVENTION

5HT receptor-specific agonists and antagonists have been used or considered for the treatment of a wide range of disorders, including anxiety, depression, hyper-tension, migraine, obesity, compulsive disorders, schizophrenia, autism, neurodegenerative disorders (e.g. Alzheimer's disease, Parkinson's disease, and Huntington's chorea), and chemotherapy-induced vomiting. See

M. D. Gershon, et al., The Peripheral Actions of 5-Hydroxytryptamine, 246 (1989); P. R. Saxena, et al., Journal of Cardiovascular Pharmacology, 15 (Supplement), 7 (1990).

Recent discoveries concerning subtype identity, distribution, structure, and function of 5HT receptors suggest the potential for novel, subtype-specific agents with greater effectiveness and fewer side effects. For example, results relevant to the treatment of male sexual dysfunction may be summarized as follows. The erection is caused by the relaxation of corpus cavernosum penis (CCP), and under normal conditions, the sympathetic nervous system more strongly affects the erectile function than does the 5HT system. Thirty years ago Gessa et al. showed that 5HT inhibits sexual function in the central nervous system. Adv. Biochem. Psychopharmacol. 1974; 11: 217-228. It is known that serotonin-releasing agents such as fenfluramine induce the penile erection through the central nervous system in rats. More recent studies have shown that the penile erection is induced by stimulation of the 5HT_{2C} receptor, but is inhibited by stimulation of 5HT_{1A} or 5HT₂ receptors. Rosen et al. described experiments indicating that selective serotonin re-uptake inhibitors (SSRIs), which allow accumulation of 5HT in the nerve endings and potentiate the effect of 5HT, cause sexual dysfunctions such as reduced sexual desire (libido) and orgasm and ejaculatory and erectile dysfunctions. J Clin. Psychopharmacol. 1999; 19: 67-85. However, 5HT_{2A} receptor antagonists have been shown to counteract erectile dysfunction by antagonizing the effect of serotonin on CCP, suggesting a therapeutic role for compounds combining SSRIs and 5HT_{2A} antagonists for treating depression without causing sexual dysfunction.

U.S. Pat. No. 4,018,830, issued Apr. 19, 1997, refers to phenylthioaralkylamines and 2-phenylthiobenzylamines which are active as antiarrhythmics. WO 97/17325, published May 15, 1997, refers to derivatives of N,N-dimethyl-2-(arylthio)benzylamine which selectively influence serotonin transport in the central nervous system and are useful as antidepressants. U.S. Pat. No. 5,190,965, issued Mar. 2, 1993, and U.S. Pat. No. 5,430,063, issued Jul. 4, 1995, refer to phenoxyphenyl derivatives which have utility in the treatment of depression.

WO01/72687, published Oct. 4, 2001, refers to biaryl ethers which inhibit monoamine reuptake and exhibit selective serotonin reuptake activity. U.S. Pat. No. 4,161,529, issued Jul. 17, 1979, refers to pyrrolidine derivatives that possess anticholesteremic and hypolipemic activity. U.S. Pat. No. 6,410,736, issued Jun. 25, 2002, and U.S. Pat. No. 6,677,378, issued Jan. 13, 2004, refer to biaryl ethers that have activity in inhibiting reuptake of serotonin, norepinephrine and/or dopamine.

This invention relates to novel diaryl ether derivatives that exhibit activity as 5HT2 antagonists, including 5HT2A and 5HT2C subtypes, to pharmaceutical compositions containing such compounds and to methods of using such compounds to treat central nervous system (CNS) and other disorders associated with 5HT receptors.

### SUMMARY OF THE INVENTION

The present invention provides a 5HT2 antagonist having the formula Ia, Ib or Ic: 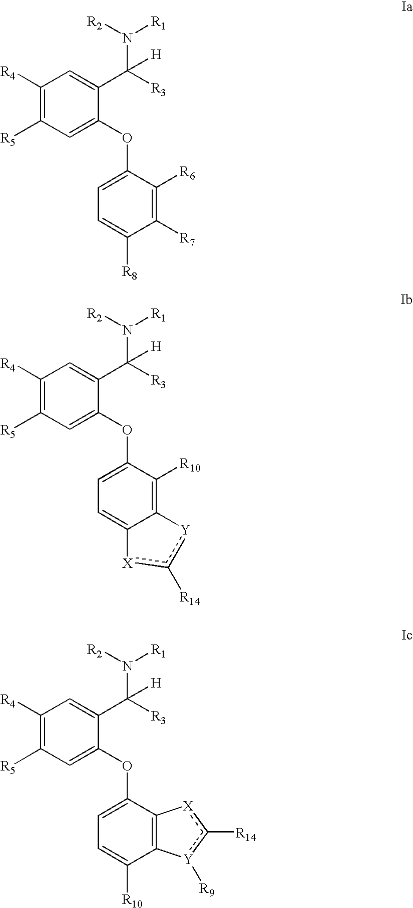
wherein X and Y are independently O, O(CH₂)ₙ, S, S(CH₂)ₙ, N, NR₁₈, NR₁₈N, NR₁₈(CH₂)ₙ, CR₁₈R₁₉(CH₂)ₙ or (CR₁₈R₁₉)ₖ, where R₁₈ and R₁₉ are independently H, straight chain or branched C₁-C₆ alkyl, CF₃, CN;

R₁, R₂ and R₃ are, independently, H or CH₃;

R₄ is H, F, Cl or CH₃;

R₅ is F, Cl, Br, C₁-C₆ alkyl, (CH₂)ₙCN, (CH₂)ₙOH, (CH₂)ₙCO₂Et, C₁-C₆ cycloalkyl, oxazolyl or substituted oxazolyl, CR₁₁R₁₂—(CH₂)ₙCH₃, or S(O)ₘ—(CH₂)ₚCH₃;

R₆ is H, F, Cl, Br, O(CH₂)ᵣCH₃, C₁-C₆ alkyl, or CN;

R₇ is H, F, Cl, Br, C₁-C₆ alkyl, O—(CH₂)ₛCH₃, Cl, CN, N(R₁₃)(R₁₅), or OH;

R₈ is H, F, Cl, Br, C₁-C₆ alkyl, O—(C₁-C₆ alkyl), S—(C₁-C₆ alkyl), OH, NH—R₁₆, or S(O)ₜ—(C₁-C₆) alkyl;

R₉ is H, CH₃, OH, or, if Y is C, R₉ may alternatively be ═O;

R₁₀ is H, Cl, F, Br, C₁-C₆ alkyl, O—(C₁-C₆ alkyl), S—(C₁-C₆ alkyl), OH, NH—R₁₇, or S(O)ᵤ—(C₁-C₆ alkyl); or, R₆ and R₇, or R₇ and R₈, or R₉ and R₁₀, together with the atoms to which they are attached, form a 5- to 8-membered ring containing one or more heteroatoms selected from the group consisting of N, O, and S;

R₁₁ and R₁₂ are, independently, H, OH, O—(C₁-C₆ alkyl), C₁-C₆ alkyl, S(O)ᵥ—(C₁-C₆ alkyl), CO—NH—(C₁-C₆ alkyl), O—(C₁-C₆ alkyl), (CH₂)ₙ—S(O)ₘ—(C₁-C₆ alkyl), or CO—NH-aryl;

R₁₃, R₁₅, R₁₆, and R₁₇ are, independently, H, or C₁-C₆ alkyl;

R₁₄ is H, CH₃, Cl, OH, O, O—(C₁-C₆ alkyl), NH₂, NHCH₃, or ═O;

k is 1 or 2; m, u, and v are, independently, 0, 1, or 2; n, p, q, r, s, and t are, independently, 0, 1, 2, 3, 4, 5, or 6; and, the dashed line represents an optional double bond; or, a pharmaceutically acceptable salt thereof. In one aspect of the invention, R₁, R₂, and R₃ are, independently, hydrogen or methyl. In another aspect of the invention, R₁, R₂, and R₃ are, independently, hydrogen or methyl; and, R₇ is O(CH₂)ᵣCH₃. In another aspect of the invention, R₄ is hydrogen, fluoro, or methyl; and R₅ is CR₁₁R₁₂—(CH₂)ₙCH₃.

In another aspect of the invention, R₁ and R₃ are both hydrogen; and, R₂ is methyl. In another aspect of the invention, R₁₁ and R₁₂ are, independently, H, OH, CO—NH—(C₁-C₆ alkyl), CO—NH-aryl, or O—(C₁-C₆ alkyl), wherein R₈ may be fluoro. In another aspect of the invention, R₇ is OCH₃, wherein R₁ and R₃ may both be hydrogen, and R₂ is methyl. Additionally, in another aspect of the invention, R₄ and R₅ are, independently, halogens. In yet another aspect of the invention, R₁ and R₃ are both hydrogen, R₂ is methyl, R₅ is a halogen, and, R₄ is not hydrogen. In a particular aspect, R₄ is fluoro or methyl.

In another aspect of the invention, R₁ and R₃ are both hydrogen; R₂ is methyl; R₄ is fluoro, methyl, or hydrogen; and, R₅ is methyl or CR₁₁R₁₂—(CH₂)ₙCH₃, and in a more particular aspect, R₄ is fluoro and R₅ is chloro.

In certain aspects of the invention, the 5HT2 antagonist is a 5HT2A or 5HT2C antagonist.

Specific embodiments of the 5HT2 antagonist of the invention include:

[4-Chloro-5-fluoro-2-(3-methoxy-2-methylphenoxy)benzyl]methylamine, hydrochloride salt;

3-(5-Chloro-4-fluoro-2-methylaminomethylphenoxy)-2-methylphenol;

[4-Bromo-2-(3-methoxy-2-methylphenoxy)benzyl]methylamine;

2-[3-(3-Methoxy-2-methylphenoxy)-4-methylaminomethylphenyl]propan-2-ol;

[4-Chloro-2-(3-methoxy-2-methylphenoxy)-5-methylbenzyl]methylamine;

[2-(4-Chlorophenoxy)-4-methanesulfonylbenzyl]methylamine;

1-[3-(4-Chloro-3-methoxy-2-methylphenoxy)-4-methylaminomethylphenyl]propan-1-ol;

[4-Chloro-2-(1-methyl-1H-indol-4-yloxy)benzyl]methylamine;

[4-Chloro-2-(4-chloro-3-methoxy-2-methylphenoxy)-5-fluorobenzyl]methylamine;

[4-Bromo-2-(4-chloro-3-methoxy-2-methylphenoxy)benzyl]methylamine;

6-Chloro-3-(5-chloro-4-fluoro-2-methylaminomethylphenoxy)-2-methylphenol;

[4-Chloro-2-(3-methoxy-2-methylphenoxy)benzyl]methylamine;

[4-Bromo-2-(3-methoxy-2-methylphenoxy)-5-methylbenzyl]methylamine;

1-[3-(4-Chloro-2,3-dimethylphenoxy)-4-methylaminomethylphenyl]propan-1-ol;

[4-Chloro-2-(4-chloro-2,3-dimethylphenoxy)-5-fluorobenzyl]methylamine;

[2-(4-Chloro-2,3-dimethylphenoxy)-4-methanesulfonylbenzyl]methylamine;

[4-Chloro-2-(4-chloro-2,3-dimethylphenoxy)-5-methylbenzyl]methylamine;

[4-Bromo-2-(4-chloro-2,3-dimethylphenoxy)benzyl]methylamine;

[2-(4-Chloro-2,3-dimethylphenoxy)-4-methanesulfonyl-5-methylbenzyl]methylamine;

[2-(4-Chloro-2,3-dimethylphenoxy)-4-methanesulfinyl-5-methylbenzyl]methylamine;

[4-Chloro-2-(4-chloro-2,3-dimethylphenoxy)benzyl]methylamine;

1-[3-(4-Chloro-2,3-dimethylphenoxy)-4-methylaminomethylphenyl]ethanol;

[3-(4-Chloro-2,3-dimethylphenoxy)-4-methylaminomethylphenyl]methanol;

[2-(4-Chloro-2,3-dimethylphenoxy)-4-(pyrrolidine-1-sulfonyl)benzyl]methylamine;

[2-(4-Chloro-2,3-dimethylphenoxy)-4-methoxymethylbenzyl]methylamine;

[2-(4-Chloro-2,3-dimethylphenoxy)-4-(1-methoxypropyl)benzyl]methylamine;

[2-(4-Chloro-2,3-dimethylphenoxy)-4-(1-methoxyethyl)benzyl]methylamine;

[4-Chloro-2-(4-chloro-2,3-dimethylphenoxy)-5-fluorobenzyl]methylamine;

1-[3-(4-Chloro-2,3-dimethylphenoxy)-4-methylaminomethylphenyl]prop-2-yn-1-ol;

1-[3-(4-Chloro-2,3-dimethylphenoxy)-4-methylaminomethylphenyl]-pent-4-en-1-ol;

1-[3-(4-Chloro-2,3-dimethylphenoxy)-4-methylaminomethylphenyl]-3-phenyl-prop-2-yn-1-ol; and,

[3-(4-Chloro-2,3-dimethylphenoxy)-4-methylaminomethylphenyl]-(2-methoxy-phenyl)-methanol.

Other specific embodiments of the 5HT2 antagonist of the invention include:

(S)-1-[2-Fluoro-5-(7-fluoroindan-4-yloxy)-4-methylaminomethylphenyl]propan-1-ol;

1-[3-(2-Chloro-4-fluoro-3-methylphenoxy)-4-methylaminomethylphenyl]propan-1-ol;

1-[3-(2-Chloro-4-fluoro-3-methylphenoxy)-4-methylaminomethylphenyl]propan-1-ol;

1-[3-(4-Chloro-2,3-dimethylphenoxy)-4-methylaminomethylphenyl]-2-methyl-prop-2-en-1-ol;

1-[3-(4-Chloro-2,3-dimethylphenoxy)-4-methylaminomethylphenyl]but-3-en-1-ol;

[3-(4-Chloro-2,3-dimethylphenoxy)-4-methylaminomethylphenyl](3-fluorophenyl)-methanol;

1-[3-(3-Methoxy-2-methylphenoxy)-4-methylaminomethylphenyl]propan-1-ol;

[2-(3-Methoxy-2-methylphenoxy)-4-(1-methoxypropyl)benzyl]methylamine;

[2-(4-Chloro-3-methoxy-2-methylphenoxy)-4-(1-methoxypropyl)benzyl]methylamine;

[4-Chloro-2-(4-chloro-2-fluorophenoxy)benzyl]methylamine;

[4-Chloro-2-(4-chloro-2-fluorophenoxy)-5-fluorobenzyl]methylamine;

[4-Chloro-2-(4-chloro-2-fluorophenoxy)-5-fluorobenzyl]dimethylamine;

[2-(4-Chloro-2-fluorophenoxy)-5-fluoro-4-methylbenzyl]methylamine;

{1-[4-Chloro-2-(4-chloro-2-fluorophenoxy)-5-fluorophenyl]ethyl}methylamine;

{1-[4-Chloro-2-(4-chloro-2-fluorophenoxy)-5-fluorophenyl]ethyl}dimethylamine;

[4-Chloro-2-(4-chloro-2-fluorophenoxy)-5-methylbenzyl]methylamine;

[5-Chloro-2-(4-chloro-2-fluorophenoxy)-4-methylbenzyl]methylamine;

[2-(4-Chloro-2-fluorophenoxy)-4,5-dimethylbenzyl]methylamine;

[2-(4-Chloro-2-fluorophenoxy)-4-methoxy-benzyl]methylamine;

[2-(4-Chloro-2-fluorophenoxy)-4-methylbenzyl]methylamine;

[2-(4-Chloro-2-fluorophenoxy)-5-fluoro-4-propylsulfanylbenzyl]methylamine;

[2-(4-Chloro-2-fluorophenoxy)-5-fluoro-4-isopropylsulfanylbenzyl]methylamine;

[4-Bromo-2-(4-chloro-2-fluorophenoxy)benzyl]methylamine;

[4-Chloro-2-(4-chloro-2-fluoro-3-methylphenoxy)-5-fluorobenzyl]methylamine;

[2-(4-Chloro-2-fluorophenoxy)-5-fluoro-4-methanesulfonylbenzyl]methylamine;

[4-(Butane-1-sulfonyl)-2-(4-chloro-2-fluorophenoxy)-5-fluorobenzyl]methylamine;

[2-(4-Chloro-2-fluorophenoxy)-5-fluoro-4-(propane-1-sulfonyl)benzyl]methylamine;

[2-(4-Chloro-2-fluoro-3-methylphenoxy)-4-methanesulfonylbenzyl]methylamine;

1-[3-(4-Chloro-2-fluorophenoxy)-4-methylaminomethylphenyl]propan-1-ol;

[4-Chloro-2-(1,3-dihydrobenzo[c]thiophen-5-yloxy)benzyl]methylamine;

[4-Chloro-2-(2,3-dihydrobenzo[b]thiophen-6-yloxy)benzyl]methylamine;

[4-Chloro-2-(2,3-dihydrobenzo[b]thiophen-5-yloxy)benzyl]methylamine;

[4-Chloro-2-(1,3-dihydro-isobenzofuran-5-yloxy)benzyl]methylamine;

[4-Chloro-2-(7-fluoroindan-4-yloxy)benzyl]methylamine;

[2-(Indan-5-yloxy)-4-methanesulfonylbenzyl]methylamine;

[4-Methanesulfonyl-2-(naphthalen-2-yloxy)benzyl]methylamine;

[4-Chloro-2-(1-methyl-1,2,3,4-tetrahydro-quinolin-6-yloxy)benzyl]methylamine;

[2-(4-Chlorophenoxy)-4-ethylsulfanylbenzyl]methylamine;

4-(5-Chloro-4-fluoro-2-methylaminomethylphenoxy)-2-methylphenol;

[4-Bromo-2-(1,4-dimethyl-1H-indol-5-yloxy)benzyl]methylamine;

[4-Chloro-2-(1,4-dimethyl-2,3-dihydro-1H-indol-5-yloxy)-5-fluorobenzyl]methylamine; and,

[4-Chloro-2-(1,4-dimethyl-1H-indol-5-yloxy)-5-methylbenzyl]methylamine.

In another aspect, the invention provides a pharmaceutical composition for use in treating a disorder or condition in a mammal selected from depression, anxiety, depression with concomitant anxiety, dysthymia, post traumatic stress disorder, panic phobias, obsessive compulsive disorder (OCD), OCD with comorbid Tourette's Syndrome, borderline personality disorder, sleep disorder, psychosis, seizures, dyskinesis, symptoms of Huntington's or Parkinson's diseases, spasticity, suppression of seizures resulting from epilepsy, cerebral ischemia, anorexia, faintness attacks, hypokinesia, cranial traumas, chemical dependencies, premature ejaculation, premenstrual syndrome (PMS) associated mood and appetite disorder, inflammatory bowel disease, modification of feeding behavior, blocking carbohydrate cravings, late luteal phase dysphoric disorder, tobacco withdrawal-associated symptoms, panic disorder, bipolar disorder, sleep disorders, jet lag, cognitive dysfunction, hypertension, bulimia, anorexia, obesity, cardiac arrhythmias, chemical dependencies and addictions selected from dependencies on, or addictions to nicotine or tobacco products, alcohol, benzodiazepines, barbiturates, opioids or cocaine; pathological gambling; trichotilomania; headache, stroke, traumatic brain injury (TBI), psychosis, Huntington's Chorea, tardive dyskinesia, hyperkinesia, dyslexia, schizophrenia, multi-infarct dementia, epilepsy, senile dementia of the Alzheimer's type (AD), Parkinson's disease (PD), attention deficit hyperactivity disorder (ADHD) and Tourette's Syndrome, comprising an amount of the 5HT2 antagonist of the invention, or a pharmaceutically acceptable salt thereof, that is effective in treating such disorder or condition and a pharmaceutically acceptable carrier.

In another aspect, the invention provides a method of treating a disorder or condition in a mammal selected from depression, anxiety, depression with concomitant anxiety, dysthymia, post traumatic stress disorder, panic phobias, obsessive compulsive disorder (OCD), OCD with comorbid Tourette's Syndrome, borderline personality disorder, sleep disorder, psychosis, seizures, dyskinesis, symptoms of Huntington's or Parkinson's diseases, spasticity, suppression of seizures resulting from epilepsy, cerebral ischemia, anorexia, faintness attacks, hypokinesia, cranial traumas, chemical dependencies, premature ejaculation, premenstrual syndrome (PMS) associated mood and appetite disorder, inflammatory bowel disease, modification of feeding behavior, blocking carbohydrate cravings, late luteal phase dysphoric disorder, tobacco withdrawal-associated symptoms, panic disorder, bipolar disorder, sleep disorders, jet lag, cognitive dysfunction, hypertension, bulimia, anorexia, obesity, cardiac arrhythmias, chemical dependencies and addictions selected from dependencies on, or addictions to nicotine or tobacco products, alcohol, benzodiazepines, barbiturates, opioids or cocaine; pathological gambling; trichotilomania; headache, stroke, traumatic brain injury (TBI), psychosis, Huntington's Chorea, tardive dyskinesia, hyperkinesia, dyslexia, schizophrenia, multi-infarct dementia, epilepsy, senile dementia of the Alzheimer's type (AD), Parkinson's disease (PD), attention deficit hyperactivity disorder (ADHD) and Tourette's Syndrome, comprising administering to a mammal in need of such treatment an amount of a 5HT2 antagonist of the invention, or a pharmaceutically acceptable salt thereof, that is effective in treating such disorder or condition.

In another aspect, the invention provides a pharmaceutical composition for use in treating a disorder or condition in a mammal selected from depression, anxiety, depression with concomitant anxiety, dysthymia, post traumatic stress disorder, panic phobias, obsessive compulsive disorder (OCD), OCD with comorbid Tourette's Syndrome, borderline personality disorder, sleep disorder, psychosis, seizures, dyskinesis, symptoms of Huntington's or Parkinson's diseases, spasticity, suppression of seizures resulting from epilepsy, cerebral ischemia, anorexia, faintness attacks, hypokinesia, cranial traumas, chemical dependencies, premature ejaculation, premenstrual syndrome (PMS) associated mood and appetite disorder, inflammatory bowel disease, modification of feeding behavior, blocking carbohydrate cravings, late luteal phase dysphoric disorder, tobacco withdrawal-associated symptoms, panic disorder, bipolar disorder, sleep disorders, jet lag, cognitive dysfunction, hypertension, bulimia, anorexia, obesity, cardiac arrhythmias, chemical dependencies and addictions selected from dependencies on, or addictions to nicotine or tobacco products, alcohol, benzodiazepines, barbiturates, opioids or cocaine; pathological gambling; trichotilomania; headache, stroke, traumatic brain injury (TBI), psychosis, Huntington's Chorea, tardive dyskinesia, hyperkinesia, dyslexia, schizophrenia, multi-infarct dementia, epilepsy, senile dementia of the Alzheimer's type (AD), Parkinson's disease (PD), attention deficit hyperactivity disorder (ADHD) and Tourette's Syndrome, comprising an amount of a 5HT2 antagonist of the invention that is an effective to ameliorate said disorder or condition by modulation of a 5HT function and a pharmaceutically acceptable carrier.

The present invention also provides a compound having the formula Ia, Ib or Ic: 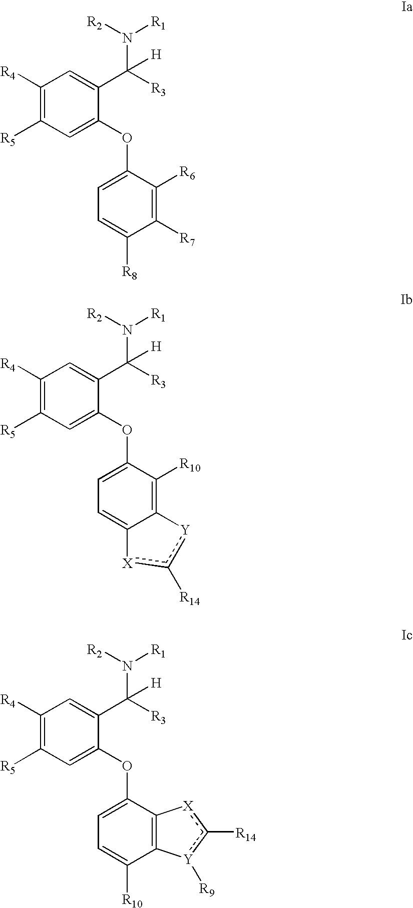
wherein X and Y are independently O, O(CH₂)ₙ, S, S(CH₂)ₙ, N, NR₁₈, NR₁₈N, NR₁₈(CH₂)ₙ, CR₁₈R₁₉(CH₂)ₙ or (CR₁₈R₁₉)ₖ, where R₁₈ and R₁₉ are independently H, straight chain or branched C₁-C₆ alkyl, CF₃, CN;

R₁, R₂ and R₃ are, independently, H or CH₃;

R₄ is H, F, Cl or CH₃;

R₅ is F, Cl, Br, C₁-C₆ alkyl, (CH₂)ₙCN, (CH₂)ₙOH, (CH₂)ₙCO₂Et, C₁-C₆ cycloalkyl, oxazolyl or substituted oxazolyl, CR₁₁R₁₂—(CH₂)ₙCH₃, or S(O)ₘ—(CH₂)ₚCH₃;

R₆ is H, F, Cl, Br, O(CH₂)ᵣCH₃, C₁-C₆ alkyl, or CN;

R₇ is H, F, Cl, Br, C₁-C₆ alkyl, O—(CH₂)ₛCH₃, Cl, CN, N(R₁₃)(R₁₅), or OH;

R₈ is H, F, Cl, Br, C₁-C₆ alkyl, O—(C₁-C₆ alkyl), S—(C₁-C₆ alkyl), OH, NH—R₁₆, or S(O)ₜ—(C₁-C₆) alkyl; with the proviso that when R₄ is H, then only one of R₆, R₇ and R₈ may be H, and no two of R₆, R₇ and R₈ are the same;

R₉ is H, CH₃, OH, or, if Y is C, R₉ may alternatively be ═O;

R₁₀ is H, Cl, F, Br, C₁-C₆ alkyl, O—(C₁-C₆ alkyl), S—(C₁-C₆ alkyl), OH, NH—R₁₇, or S(O)ᵤ—(C₁-C₆ alkyl); or, R₆ and R₇, or R₇ and R₈, or R₉ and R₁₀, together with the atoms to which they are attached, form a 5- to 8-membered ring containing one or more heteroatoms selected from the group consisting of N, O, and S;

R₁₁ and R₁₂ are, independently, H, OH, O—(C₁-C₆ alkyl), C₁-C₆ alkyl, S(O)ᵥ—(C₁-C₆ alkyl), CO—NH—(C₁-C₆ alkyl), O—(C₁-C₆ alkyl), (CH₂)ₙ—S(O)ₘ—(C₁-C₆ alkyl), or CO—NH-aryl;

R₁₃, R₁₅, R₁₆, and R₁₇ are, independently, H, or C₁-C₆ alkyl;

R₁₄ is H, CH₃, Cl, OH, O, O—(C₁-C₆ alkyl), NH₂, NHCH₃, or ═O;

k is 1 or 2; m, u, and v are, independently, 0, 1, or 2; n, p, q, r, s, and t are, independently, 0, 1, 2, 3, 4, 5, or 6; and, the dashed line represents an optional double bond; or, a pharmaceutically acceptable salt thereof. In one aspect of the invention, R₁, R₂, and R₃ are, independently, hydrogen or methyl. In another aspect of the invention, R₁, R₂, and R₃ are, independently, hydrogen or methyl; and, R₇ is O(CH₂)ᵣCH₃. In another aspect of the invention, R₄ is hydrogen, fluoro, or methyl; and R₅ is CR₁₁R₁₂—(CH₂)ₙCH₃.

In another aspect of the invention, R₁ and R₃ are both hydrogen; and, R₂ is methyl. In another aspect of the invention, R₁₁ and R₁₂ are, independently, H, OH, CO—NH—(C₁-C₆ alkyl), CO—NH-aryl, or O—(C₁-C₆ alkyl), wherein R₈ may be fluoro. In another aspect of the invention, R₇ is OCH₃, wherein R₁ and R₃ may both be hydrogen, and R₂ is methyl. Additionally, in another aspect of the invention, R₄ and R₅ are, independently, halogens. In yet another aspect of the invention, R₁ and R₃ are both hydrogen, R₂ is methyl, R₅ is a halogen, and, R₄ is not hydrogen. In a particular aspect, R₄ is fluoro or methyl.

In another aspect of the invention, R₁ and R₃ are both hydrogen; R₂ is methyl; R₄ is fluoro, methyl, or hydrogen; and, R₅ is methyl or CR₁₁R₁₂—(CH₂)ₙCH₃, and in a more particular aspect, R₄ is fluoro and R₅ is chloro.

In certain aspects of the invention, the compound is a 5HT2A or 5HT2C antagonist.

The invention further provides a compound selected from the group consisting of:

[4-Chloro-5-fluoro-2-(3-methoxy-2-methylphenoxy)benzyl]methylamine, hydrochloride salt;

3-(5-Chloro-4-fluoro-2-methylaminomethylphenoxy)-2-methylphenol;

[4-Bromo-2-(3-methoxy-2-methylphenoxy)benzyl]methylamine;

2-[3-(3-Methoxy-2-methylphenoxy)-4-methylaminomethylphenyl]propan-2-ol;

[4-Chloro-2-(3-methoxy-2-methylphenoxy)-5-methylbenzyl]methylamine;

[2-(4-Chlorophenoxy)-4-methanesulfonylbenzyl]methylamine;

1-[3-(4-Chloro-3-methoxy-2-methylphenoxy)-4-methylaminomethylphenyl]propan-1-ol;

[4-Chloro-2-(1-methyl-1H-indol-4-yloxy)benzyl]methylamine;

[4-Chloro-2-(4-chloro-3-methoxy-2-methylphenoxy)-5-fluorobenzyl]methylamine;

[4-Bromo-2-(4-chloro-3-methoxy-2-methylphenoxy)benzyl]methylamine;

6-Chloro-3-(5-chloro-4-fluoro-2-methylaminomethylphenoxy)-2-methylphenol;

[4-Chloro-2-(3-methoxy-2-methylphenoxy)benzyl]methylamine;

[4-Bromo-2-(3-methoxy-2-methylphenoxy)-5-methylbenzyl]methylamine;

1-[3-(4-Chloro-2,3-dimethylphenoxy)-4-methylaminomethylphenyl]propan-1-ol;

[4-Chloro-2-(4-chloro-2,3-dimethylphenoxy)-5-fluorobenzyl]methylamine;

[2-(4-Chloro-2,3-dimethylphenoxy)-4-methanesulfonylbenzyl]methylamine;

[4-Chloro-2-(4-chloro-2,3-dimethylphenoxy)-5-methylbenzyl]methylamine;

[4-Bromo-2-(4-chloro-2,3-dimethylphenoxy)benzyl]methylamine;

[2-(4-Chloro-2,3-dimethylphenoxy)-4-methanesulfonyl-5-methylbenzyl]methylamine;

[2-(4-Chloro-2,3-dimethylphenoxy)-4-methanesulfinyl-5-methylbenzyl]methylamine;

[4-Chloro-2-(4-chloro-2,3-dimethylphenoxy)benzyl]methylamine;

1-[3-(4-Chloro-2,3-dimethylphenoxy)-4-methylaminomethylphenyl]ethanol;

[3-(4-Chloro-2,3-dimethylphenoxy)-4-methylaminomethylphenyl]methanol;

[2-(4-Chloro-2,3-dimethylphenoxy)-4-(pyrrolidine-1-sulfonyl)benzyl]methylamine;

[2-(4-Chloro-2,3-dimethylphenoxy)-4-methoxymethylbenzyl]methylamine;

[2-(4-Chloro-2,3-dimethylphenoxy)-4-(1-methoxypropyl)benzyl]methylamine;

[2-(4-Chloro-2,3-dimethylphenoxy)-4-(1-methoxyethyl)benzyl]methylamine;

[4-Chloro-2-(4-chloro-2,3-dimethylphenoxy)-5-fluorobenzyl]methylamine;

1-[3-(4-Chloro-2,3-dimethylphenoxy)-4-methylaminomethylphenyl]prop-2-yn-1-ol;

1-[3-(4-Chloro-2,3-dimethylphenoxy)-4-methylaminomethylphenyl]-pent-4-en-1-ol;

1-[3-(4-Chloro-2,3-dimethylphenoxy)-4-methylaminomethylphenyl]-3-phenyl-prop-2-yn-1-ol; and,

[3-(4-Chloro-2,3-dimethylphenoxy)-4-methylaminomethylphenyl]-(2-methoxy-phenyl)-methanol.

The invention also provides a compound selected from the group consisting of:

(S)-1-[2-Fluoro-5-(7-fluoroindan-4-yloxy)-4-methylaminomethylphenyl]propan-1-ol;

1-[3-(2-Chloro-4-fluoro-3-methylphenoxy)-4-methylaminomethylphenyl]propan-1-ol;

1-[3-(2-Chloro-4-fluoro-3-methylphenoxy)-4-methylaminomethylphenyl]propan-1-ol;

1-[3-(4-Chloro-2,3-dimethylphenoxy)-4-methylaminomethylphenyl]-2-methyl-prop-2-en-1-ol;

1-[3-(4-Chloro-2,3-dimethylphenoxy)-4-methylaminomethylphenyl]-but-3-en-1-ol;

[3-(4-Chloro-2,3-dimethylphenoxy)-4-methylaminomethylphenyl]-(3-fluorophenyl)-methanol;

1-[3-(3-Methoxy-2-methylphenoxy)-4-methylaminomethylphenyl]propan-1-ol;

[2-(3-Methoxy-2-methylphenoxy)-4-(1-methoxypropyl)benzyl]methylamine;

[2-(4-Chloro-3-methoxy-2-methylphenoxy)-4-(1-methoxypropyl)benzyl]methylamine;

[4-Chloro-2-(4-chloro-2-fluorophenoxy)benzyl]methylamine;

[4-Chloro-2-(4-chloro-2-fluorophenoxy)-5-fluorobenzyl]methylamine;

[4-Chloro-2-(4-chloro-2-fluorophenoxy)-5-fluorobenzyl]-dimethylamine;

[2-(4-Chloro-2-fluorophenoxy)-5-fluoro-4-methylbenzyl]methylamine;

{1-[4-Chloro-2-(4-chloro-2-fluorophenoxy)-5-fluorophenyl]ethyl}-methylamine;

{1-[4-Chloro-2-(4-chloro-2-fluorophenoxy)-5-fluorophenyl]ethyl}-dimethylamine;

[4-Chloro-2-(4-chloro-2-fluorophenoxy)-5-methylbenzyl]methylamine;

[5-Chloro-2-(4-chloro-2-fluorophenoxy)-4-methylbenzyl]methylamine;

[2-(4-Chloro-2-fluorophenoxy)-4,5-dimethylbenzyl]methylamine;

[2-(4-Chloro-2-fluorophenoxy)-4-methoxy-benzyl]methylamine;

[2-(4-Chloro-2-fluorophenoxy)-4-methylbenzyl]methylamine;

[2-(4-Chloro-2-fluorophenoxy)-5-fluoro-4-propylsulfanylbenzyl]methylamine;

[2-(4-Chloro-2-fluorophenoxy)-5-fluoro-4-isopropylsulfanylbenzyl]methylamine;

[4-Bromo-2-(4-chloro-2-fluorophenoxy)benzyl]methylamine;

[4-Chloro-2-(4-chloro-2-fluoro-3-methylphenoxy)-5-fluorobenzyl]methylamine;

[2-(4-Chloro-2-fluorophenoxy)-5-fluoro-4-methanesulfonylbenzyl]methylamine;

[4-(Butane-1-sulfonyl)-2-(4-chloro-2-fluorophenoxy)-5-fluorobenzyl]methylamine;

[2-(4-Chloro-2-fluorophenoxy)-5-fluoro-4-(propane-1-sulfonyl)benzyl]methylamine;

[2-(4-Chloro-2-fluoro-3-methylphenoxy)-4-methanesulfonylbenzyl]methylamine;

1-[3-(4-Chloro-2-fluorophenoxy)-4-methylaminomethylphenyl]propan-1-ol;

[4-Chloro-2-(1,3-dihydrobenzo[c]thiophen-5-yloxy)benzyl]methylamine;

[4-Chloro-2-(2,3-dihydrobenzo[b]thiophen-6-yloxy)benzyl]methylamine;

[4-Chloro-2-(2,3-dihydrobenzo[b]thiophen-5-yloxy)benzyl]methylamine;

[4-Chloro-2-(1,3-dihydro-isobenzofuran-5-yloxy)benzyl]methylamine;

[4-Chloro-2-(7-fluoroindan-4-yloxy)benzyl]methylamine;

[2-(Indan-5-yloxy)-4-methanesulfonylbenzyl]methylamine;

[4-Methanesulfonyl-2-(naphthalen-2-yloxy)benzyl]methylamine;

[4-Chloro-2-(1-methyl-1,2,3,4-tetrahydro-quinolin-6-yloxy)benzyl]methylamine;

[2-(4-Chlorophenoxy)-4-ethylsulfanylbenzyl]methylamine;

4-(5-Chloro-4-fluoro-2-methylaminomethylphenoxy)-2-methylphenol;

[4-Bromo-2-(1,4-dimethyl-1H-indol-5-yloxy)benzyl]methylamine;

[4-Chloro-2-(1,4-dimethyl-2,3-dihydro-1H-indol-5-yloxy)-5-fluorobenzyl]methylamine; and,

[4-Chloro-2-(1,4-dimethyl-1H-indol-5-yloxy)-5-methylbenzyl]methylamine.

In another aspect, the invention provides a pharmaceutical composition for use in treating a disorder or condition in a mammal selected from depression, anxiety, depression with concomitant anxiety, dysthymia, post traumatic stress disorder, panic phobias, obsessive compulsive disorder (OCD), OCD with comorbid Tourette's Syndrome, borderline personality disorder, sleep disorder, psychosis, seizures, dyskinesis, symptoms of Huntington's or Parkinson's diseases, spasticity, suppression of seizures resulting from epilepsy, cerebral ischemia, anorexia, faintness attacks, hypokinesia, cranial traumas, chemical dependencies, premature ejaculation, premenstrual syndrome (PMS) associated mood and appetite disorder, inflammatory bowel disease, modification of feeding behavior, blocking carbohydrate cravings, late luteal phase dysphoric disorder, tobacco withdrawal-associated symptoms, panic disorder, bipolar disorder, sleep disorders, jet lag, cognitive dysfunction, hypertension, bulimia, anorexia, obesity, cardiac arrhythmias, chemical dependencies and addictions selected from dependencies on, or addictions to nicotine or tobacco products, alcohol, benzodiazepines, barbiturates, opioids or cocaine; pathological gambling; trichotilomania; headache, stroke, traumatic brain injury (TBI), psychosis, Huntington's Chorea, tardive dyskinesia, hyperkinesia, dyslexia, schizophrenia, multi-infarct dementia, epilepsy, senile dementia of the Alzheimer's type (AD), Parkinson's disease (PD), attention deficit hyperactivity disorder (ADHD) and Tourette's Syndrome, comprising an amount of the compound having formula Ia, Ib or Ic, or a pharmaceutically acceptable salt thereof, that is effective in treating such disorder or condition and a pharmaceutically acceptable carrier.

In another aspect, the invention provides a method of treating a disorder or condition in a mammal selected from depression, anxiety, depression with concomitant anxiety, dysthymia, post traumatic stress disorder, panic phobias, obsessive compulsive disorder (OCD), OCD with comorbid Tourette's Syndrome, borderline personality disorder, sleep disorder, psychosis, seizures, dyskinesis, symptoms of Huntington's or Parkinson's diseases, spasticity, suppression of seizures resulting from epilepsy, cerebral ischemia, anorexia, faintness attacks, hypokinesia, cranial traumas, chemical dependencies, premature ejaculation, premenstrual syndrome (PMS) associated mood and appetite disorder, inflammatory bowel disease, modification of feeding behavior, blocking carbohydrate cravings, late luteal phase dysphoric disorder, tobacco withdrawal-associated symptoms, panic disorder, bipolar disorder, sleep disorders, jet lag, cognitive dysfunction, hypertension, bulimia, anorexia, obesity, cardiac arrhythmias, chemical dependencies and addictions selected from dependencies on, or addictions to nicotine or tobacco products, alcohol, benzodiazepines, barbiturates, opioids or cocaine; pathological gambling; trichotilomania; headache, stroke, traumatic brain injury (TBI), psychosis, Huntington's Chorea, tardive dyskinesia, hyperkinesia, dyslexia, schizophrenia, multi-infarct dementia, epilepsy, senile dementia of the Alzheimer's type (AD), Parkinson's disease (PD), attention deficit hyperactivity disorder (ADHD) and Tourette's Syndrome, comprising administering to a mammal in need of such treatment an amount of the compound having formula Ia, Ib or Ic, or a pharmaceutically acceptable salt thereof, that is effective in treating such disorder or condition.

The present invention further relates to a pharmaceutical composition for treating a condition or disorder that can be treated by enhancing serotonergic neurotransmission in a mammal, preferably a human, including:

a) a pharmaceutically acceptable carrier;

b) a compound of the formula Ia, Ib, or Ic or a pharmaceutically acceptable salt thereof; and,

c) a 5-HT re-uptake inhibitor, preferably sertraline, or a pharmaceutically acceptable salt thereof, or a norepinephrine reuptake inhibitor or pharmaceutically acceptable salt thereof, wherein the norepinephrine reuptake inhibitor is selected from the group consisting of racemic reboxetine, [S,S]-reboxetine, amoxapine, and maprotiline, wherein the amount of the active compounds (i.e., the compound of formula Ia, Ib, or Ic and the 5-HT re-uptake inhibitor) are such that the combination is effective in treating such disorder or condition.

The present invention also relates to a method for treating a disorder or condition that can be treated by enhancing serotonergic neurotransmission in a mammal, preferably a human, including administering to a mammal requiring such treatment:

a) a compound of the formula Ia, Ib, or Ic, defined above, or a pharmaceutically acceptable salt thereof; and,

b) a 5-HT re-uptake inhibitor, preferably sertraline, or a pharmaceutically acceptable salt thereof, or a norepinephrine reuptake inhibitor or pharmaceutically acceptable salt thereof, wherein the norepinephrine reuptake inhibitor is selected from the group consisting of racemic reboxetine, [S,S]-reboxetine, amoxapine, and maprotiline, wherein the amounts of the active compounds (i.e., the compound of formula Ia, Ib, or Ic, the 5-HT re-uptake inhibitor and the norepinephrine reuptake inhibitor ) are such that the combination is effective in treating such disorder or condition.

The present invention also relates to a method for treating a disorder or condition that can be treated by enhancing serotonergic neurotransmission in a mammal, preferably a human, including administering to the mammal requiring such treatment:

a) a 5-HT_{1A} antagonist or a pharmaceutically acceptable salt thereof; and

b) a compound of formula Ia, Ib, or Ic or a pharmaceutically acceptable salt thereof, wherein the amounts of each active compound (i.e., the 5-HT_{1A} antagonist and the compound of formula Ia, Ib, or Ic) are such that the combination is effective in treating such disorder or condition.

The present invention also relates to a pharmaceutical composition for treating a disorder or condition that can be treated by enhancing serotonergic neurotransmission in a mammal, preferably a human, including:

a) a 5-HT_{1A} antagonist or a pharmaceutically acceptable salt thereof; and

b) a compound of formula Ia, Ib, or Ic or a pharmaceutically acceptable salt thereof, wherein the amounts of each active compound (i.e., the 5-HT_{1A} antagonist and the compound of formula Ia, Ib, or Ic) are such that the combination is effective in treating such disorder or condition.

The terms “pharmaceutically acceptable salts” and “pharmaceutically acceptable acid salts” of compounds of the Formula I refer to those salts which are, within the scope of sound medical judgment, suitable for use in contact with the tissues of patients without undue toxicity, irritation, allergic response, and the like, as well as zwitterionic forms, where possible, of compounds of the invention. The compounds of Formula I are basic in nature and are thus capable of forming a wide variety of salts with various inorganic and organic acids. The acids that can be used to prepare pharmaceutically acceptable acid addition salts of those compounds of Formula I are those that form non-toxic acid addition salts, i.e., salts containing pharmacologycally acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, fumarate, gluconate, glucaronate, saccharate, formate, benzoate, glutamate, methanesulfonate, ethanesulfonate, benzenesulfonate, and p-toluenesulfonate. (See, for example, Berge, S. M., et al., “Pharmaceutical Salts,” J. Pharm. Sci. (1977) vol. 66, pp. 1-19, which is incorporated herein by reference.)

The term “one or more substituents,” as used herein, includes from one to the maximum number of substituents possible based on the number of available bonding sites.

The term “disorders of the serotonin system,” as used herein, refers to disorders the treatment of which can be effected or facilitated by altering (i.e., increasing or decreasing) serotonin-mediated neurotransmission.

The term “treating,” as used herein, refers to retarding or reversing the progress of, or alleviating or preventing either the disorder or condition to which the term “treating” applies, or one or more symptoms of such disorder or condition. The term “treatment,” as used herein, refers to the act of treating a disorder or condition, as the term “treating” is defined above.

The terms “therapeutically effective amount” and “treatment effective amount,” as used herein, refers to an amount sufficient to detectably treat, ameliorate, prevent or detectably retard the progression of an unwanted condition or symptom associated with disorders of the serotonin system.

The term “serotonin-mediated neurotransmission-altering effective amount,” as used herein, refers to an amount sufficient to increase or decrease neurotransmission in systems controlled by serotonin.

The term “modulation,” as in modulation of 5HT receptor function, refers to a fine-tuning of 5HT receptor function—either increasing or decreasing receptor function—through the use of agonists or antagonists. Such modulation is a treatment option for disorders of bodily states such as temperature, blood pressure, sleep, as well as for obesity, depression, epilepsy, anxiety, Alzheimer's disease, withdrawal from drug abuse, schizophrenia, schizophreniform disorder, schizo-affective disorder, delusional disorder, a stress-related disease (e.g. general anxiety disorder), panic disorder, a phobia, obsessive compulsive disorder, post-traumatic-stress syndrome, immune system depression, a stress-induced problem with the urinary, gastrointestinal or cardiovascular system (e.g., stress incontinence), pain disorders including neuropathic pain disorders, neurodegenerative disorders, autism, chemotherapy-induced vomiting, hypertension, migraine, headaches, cluster headaches, sexual dysfunction in a mammal (e.g. a human), addictive disorder and withdrawal syndrome, an adjustment disorder, an age-associated learning and mental disorder, anorexia nervosa, apathy, an attention-deficit disorder due to general medical conditions, attention-deficit hyper-activity disorder, behavioral disturbance (including agitation in conditions associated with diminished cognition (e.g., dementia, mental retardation or delirium)), bipolar disorder, bulimia nervosa, chronic fatigue syndrome, conduct disorder, cyclothymic disorder, dysthymic disorder, fibromyalgia and other somatoform disorders, generalized anxiety disorder, an inhalation disorder, an intoxication disorder, movement disorder (e.g., Huntington's disease or Tardive Dyskinesia), oppositional defiant disorder, peripheral neuropathy, post-traumatic stress disorder, premenstrual dysphoric disorder, a psychotic disorder (brief and long duration disorders, psychotic disorder due to medical condition, psychotic disorder NOS), mood disorder (major depressive or bipolar disorder with psychotic features) seasonal affective disorder, a sleep disorder, a specific development disorder, agitation disorder, selective serotonin reuptake inhibition (SSRI) “poop out” syndrome or a tic disorder (e.g., Tourette's syndrome).

The term “prodrug,” as used herein, refers to a chemical compound that is converted by metabolic processes in vivo to a compound of the above formula. An example of such a metabolic process is hydrolysis in blood. Thorough discussions of prodrugs are provided in the following: T. Higuchi and V. Stella, “Prodrugs as Novel Delivery Systems,” Vol. 14, ACS Symposium Series; H. Bundgaard, “Design of Prodrugs”; and “Bioreversible Carriers in Drug Design,” ed. Edward Roche, American Pharmaceutical Association and Pergamon Press, 1987, all of which are incorporated herein by reference. Preferred prodrugs for compounds of the invention include: carboxylate esters, carbonate esters, hemi-esters, phosphate esters, nitro esters, sulfate esters, sulfoxides, amides, carbamates, azo compounds, phosphamides, glycosides, ethers, acetals, and ketals.

The chemist of ordinary skill will recognize that certain combinations of substituents included within the scope of Formula I may be chemically unstable. The skilled chemist will either avoid these combinations or protect sensitive groups with well-known protecting groups. As used herein, the term “deprotecting” refers to the removal of such well-known protecting groups by methods that are well known in the art.

The term “alkyl,” as used herein, unless otherwise indicated, includes saturated monovalent hydrocarbon radicals with 1-12 carbon atoms having straight, branched or cyclic moieties or combinations thereof. The term “lower alkyl” refers to an alkyl group having one to six carbon atoms. Examples include methyl, ethyl, propyl, isopropyl, cyclopropyl, butyl, isobutyl, sec-butyl, t-butyl, pentyl, neopentyl, cyclopentylmethyl, and hexyl. It is preferred that the alkyl group is lower alkyl. The preferred cyclic alkyl groups are cyclobutyl and cyclopentyl. The preferred lower alkyl group contains 1-3 carbon atoms.

The term “alkoxy,” as used herein, unless otherwise indicated, refers to radicals having the formula —O-alkyl, wherein “alkyl” is defined as above. As used herein, the term “lower alkoxy” refers to an alkoxy group having 1-6 carbon atoms. It may be straight-chain or branched or an alkoxy-substituted alkyl group may form a cyclic ether, such as tetrahydropyran or tetrahydrofuran. Examples of acyclic alkoxy groups include methoxy, ethoxy, propoxy, isopropoxy, butoxy, isobutoxy, pentoxy and the like. It is preferred that alkoxy is lower alkoxy. It is more preferred that alkoxy contains 1-3 carbon atoms. The most preferred alkoxy group is methoxy. The most preferred substituted alkoxy group is trifluoromethoxy.

The halogen atoms contemplated by the present invention are F, Cl, Br, and I. Chlorine and fluorine are preferred. Alkyl groups substituted with one or more halogen atoms include chloromethyl, 2,3-dichloropropyl, and trifluoromethyl. It is preferred that the halo groups are the same. The most preferred halogen-substituted alkyl group is trifluoromethyl.

The term “alkenyl,” as used herein, refers to a hydrocarbon radical with two to eight carbon atoms and at least one double bond. The alkenyl group may be straight-chained, branched, or cyclic, and may be in either the Z or E form. Examples include ethenyl, 1-propenyl, 2-propenyl, 1-butenyl, 2-butenyl, isopropenyl, isobutenyl, 1-pentenyl, (Z)-2-pentenyl, (E)-2-pentenyl, (Z)-4-methyl-2-pentenyl, (E)-4-methyl-2-pentenyl, 1,3-pentadienyl, 2,4-pentadienyl, 1,3-butadienyl, cyclopentadienyl, and the like. The preferred alkenyl group is ethenyl.

The term “alkynyl” refers to a hydrocarbon radical with two to eight carbon atoms and at least one carbon-carbon triple bond. The alkynyl group may be straight chained or branched. Examples include 1-propynyl, 2-propynyl, 1-butynyl, 2-butynyl, 1-pentynyl, 2-pentynyl, 3-methyl-1-pentynyl, 3-pentynyl, 1-hexynyl, 2-hexynyl, 3-hexynyl, and the like. The preferred alkynyl group is ethynyl.

The term “aryl,” as used herein, unless otherwise indicated, includes an organic radical derived from a C₆-C₁₄ aromatic hydrocarbon by removal of one or more hydrogen(s). Examples include phenyl and naphthyl.

The term “heteroaryl,” as used herein, unless otherwise indicated, includes an organic radical derived from an aromatic heterocyclic compound by removal of one or more hydrogen atoms. The term “heterocyclic compound” denotes a ring system made up of 5-14 ring atoms and made up of carbon and at least one other element selected from the group consisting of oxygen, nitrogen, and sulfur. Examples of heteroaryl groups include benzimidazolyl, benzofuranyl, benzofurazanyl, 2H-1-benzopyranyl, benzothia-diazine, benzothiazinyl, benzothiazolyl, benzothiophenyl, benzoxazolyl, furazanyl, furopyridinyl, furyl, imidazolyl, indazolyl, indolinyl, indolizinyl, indolyl, 3H-indolyl, isoindolyl, isoquinolinyl, isothiazolyl, isoxazolyl, naphthyridinyl, oxadiazolyl, oxazolyl, phthalazinyl, pteridinyl, purinyl, pyrazinyl, pyridazinyl, pyridinyl, pyrimidinyl, pyrazolyl, pyrrolyl, quinazolinyl, quinolinyl, quinoxalinyl, tetrazolyl, thiazolyl, thiadiazolyl, thienyl, triazinyl, and triazolyl. Additional examples of heteroaryl groups include pyridyl N-oxide, pyrimidinyl N-oxide, pyridazinyl N-oxide, quinolinyl N-oxide, and isoquinolyl N-oxide.

Certain compounds of Formula Ia, Ib or Ic contain one or more chiral centers and therefore exist in different enantiomeric and diasteriomeric forms. As defined above, Formula I includes—and this invention relates to the use of—all optical isomers and other stereoisomers of compounds of the Formula I and mixtures thereof. It is to be understood that the present invention encompasses any racemic, optically active, polymorphic, tautomeric, or stereoisomeric form, or mixture thereof, of a compound of the invention, which possesses the useful properties described herein. Where compounds of this invention exist in different tautomeric forms, this invention relates to all tautomers of Formula I.

### DETAILED DESCRIPTION OF THE INVENTION

The 5HT2 antagonists and compounds of the invention can be prepared in a variety of ways, as shown below in Schemes 1-4. In the following reaction schemes and hereafter, unless otherwise stated, R¹ to R¹⁷, X, Y n, m, and p are as defined above. These processes form further embodiments of the invention.

Throughout the specification, general formulae are designated by Roman numerals I, II, III, IV, V, etc. Subsets of these general formulae are defined as Ia, Ib, Ic, etc. Designations such as A1, B1, A2B2, A1B2, A2B2, etc. are used to describe various A and B subunits and combinations thereof, where A and B subunits are the various substituted A and B rings as shown in the generic formulas. Commonly used abbreviations and acronyms for organic reagents include DMF for N,N-dimethylformamide, THF for tetrahydrofuran, LDA for lithium diisopropylamide, DCM for dichloromethane.

In any of the following synthetic sequences, it may be determined by the skilled chemist that it is necessary and/or desirable to protect sensitive or reactive groups on any of the molecules concerned. This may be achieved by means of conventional protecting groups, such as those described in T. W. Greene and P. G. M. Wuts, Protective Groups in Organic Synthesis, John Wiley & Sons, 1999.

The 5HT2 antagonists and compounds of the invention can generally be prepared as illustrated in Scheme 1.

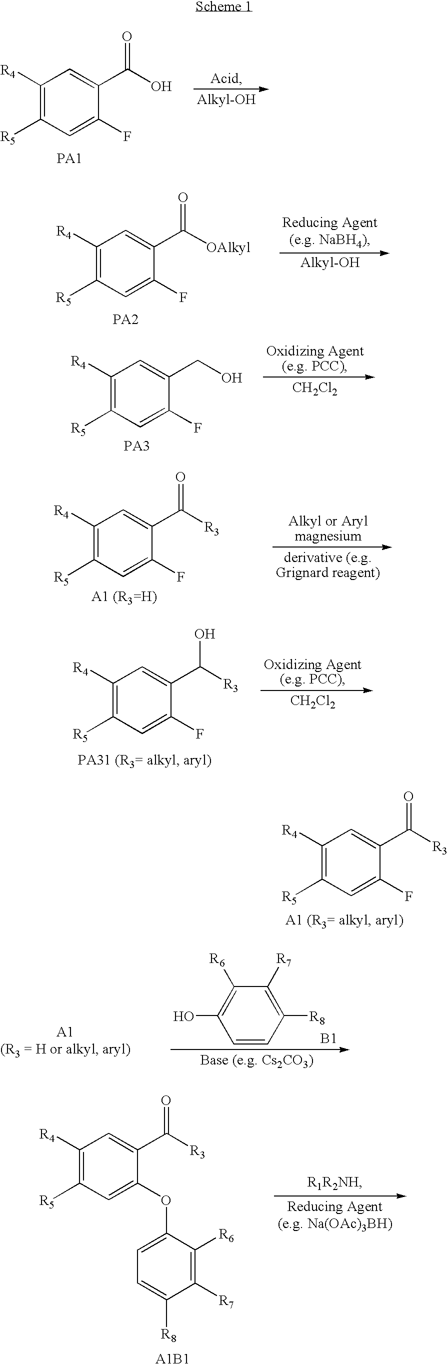

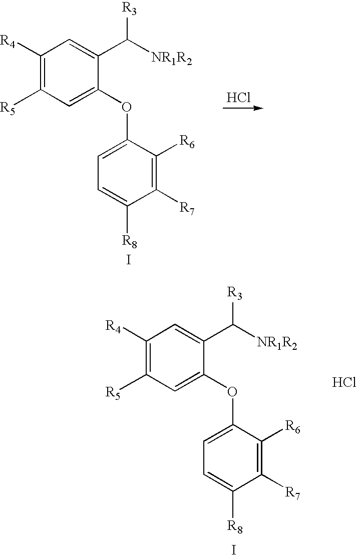

As shown in Scheme 1, a compound of general formula A1B1, a precursor to Formula I, can be prepared by the reaction of a compound with general formula A1 with a compound of general formula B1 in presence of a base such as sodium hydride, potassium carbonate, cesium carbonate and triethylamine in solvents such as DMF, THF, acetonitrile, C₁-C₆ alkyl alcohols, or mixtures thereof, at temperatures ranging from ambient to the boiling point of said mixtures. In turn, a compound of general formula I can be prepared, according to methods well known in the art, by the reaction of a compound A1B1 with a primary or secondary amine NHR¹R² in the presence of a reducing agent. The typical reducing agent would be a borohydride derivative such as sodium triacetoxy borohydride, sodium cyanoborohydride, or sodium borohydride. Typical solvents for this reaction are DCM, 1,2-dichloromethane, methanol, ethanol, or THF, or mixtures thereof. (See, for example, Lane, C. F., “Sodium Cyanoborohydride—A Highly Selective Reducing Agent for Organic Functional Groups,” Synthesis, 1975, 135). The compounds included in claim 3 and 4 above can also be prepared by the route described in Scheme 1. In some cases the B-ring will require an appropriate protecting group prior to the coupling procedure to make the diaryl ether (A1B1) with deprotection occurring after the alkyl amino group is installed. An example of this is provided in Scheme 5 below.

Scheme 1 also shows the preparation of starting material A1 from commercially available precursors PA1 and PA2 by esterification of an appropriately substituted ortho fluoro benzoic acid derivative PA1. The esterification takes place in the presence of an acid, in an alcohol solvent, at temperature ranging from ambient to the boiling point of the solvent. The ester derivative PA2 is then reduced to a benzyl alcohol derivative PA3 with a reducing agent such as, but not limited to, sodium borohydride in solvents such as alcohols, and the benzyl alcohol derivative PA3 is then oxidized with a reagent such, but not limited to PCC in an inert organic solvent such as methylene chloride, at temperatures ranging from ambient temperature to the boiling point of said solvent, to a benzaldehyde derivate with general formula A1 where R³ is hydrogen.

Compounds of general formula A1 where R³ is hydrogen can be converted to a secondary benzyl alcohol derivative of general formula PA31 with an alkyl or aryl magnesium derivative in aprotic solvents such as diethyl ether, THF, toluene or similar solvents and mixtures thereof, at temperatures ranging from about −80° C. to ambient temperature. The benzyl alcohol derivative PA31 is then oxidized in an inert organic solvent such as dichloromethane (methylene chloride), at temperatures ranging from ambient temperature to the boiling point of said solvent, to produce a compound with general formula A1, where R³ is not hydrogen.

Many alternative methods are available for the preparation of compounds of general formula A1. Examples include the reaction (in THF, ether, or hexane) of a suitably substituted fluorobenzene derivative with either i) an alkyl lithium derivative to provide compounds with general formula A1 where R³=hydrogen, or ii) an alkyl or aryl aldehyde derivative in the presence of a metal salt such as anhydrous ZnCl₂, to provide compounds with general formula A1 where R³ is not hydrogen. Scheme 1a shows one variation.

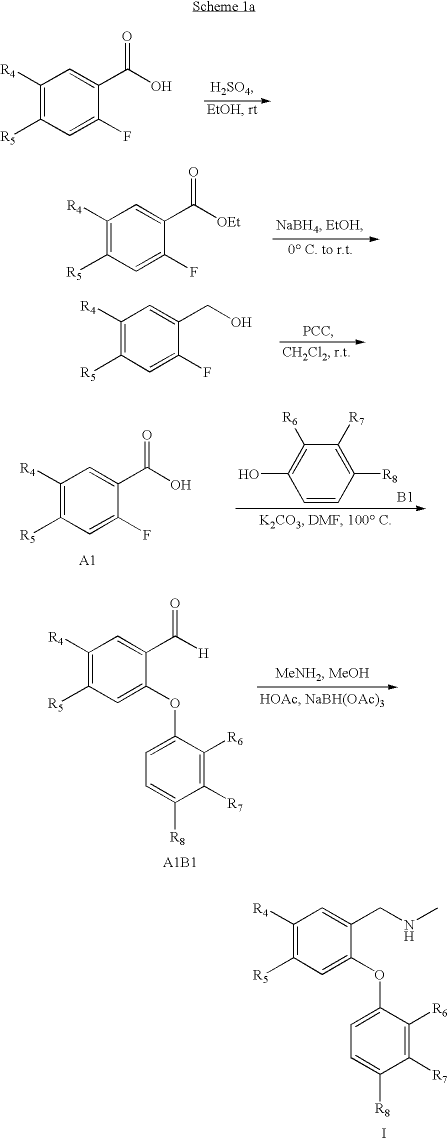

Starting materials A1 and B1 are commercially available or can be prepared by procedures that are well known to one of ordinary skill in organic chemistry.

The variables in Scheme 1a are as defined above or as in the claims. In the final step, standard reductive alkylation conditions can be used to derivatize a secondary amine. As is well known in the art, such alkylation conditions involve treatment with an aldehyde in the presence of sodium triacetoxyborohydride (see, for example, Abdel-Magid, A. F., et al, “Reductive Amination of Aldehydes and Ketones with Sodium Triacetoxyborohydride. Studies on Direct and Indirect Reductive Amination Procedures,” Journal of Organic Chemistry, 1996, v61, 3849-62.) or sodium cyanoborohydride (see, for example, Lane, C. F., “Sodium Cyanoborohydride—A Highly Selective Reducing Agent for Organic Functional Groups,” Synthesis, 1975, 135).

Compounds wherein R⁵ is other than H and Cl can also be prepared according to procedures shown in another variation, Scheme 1b.

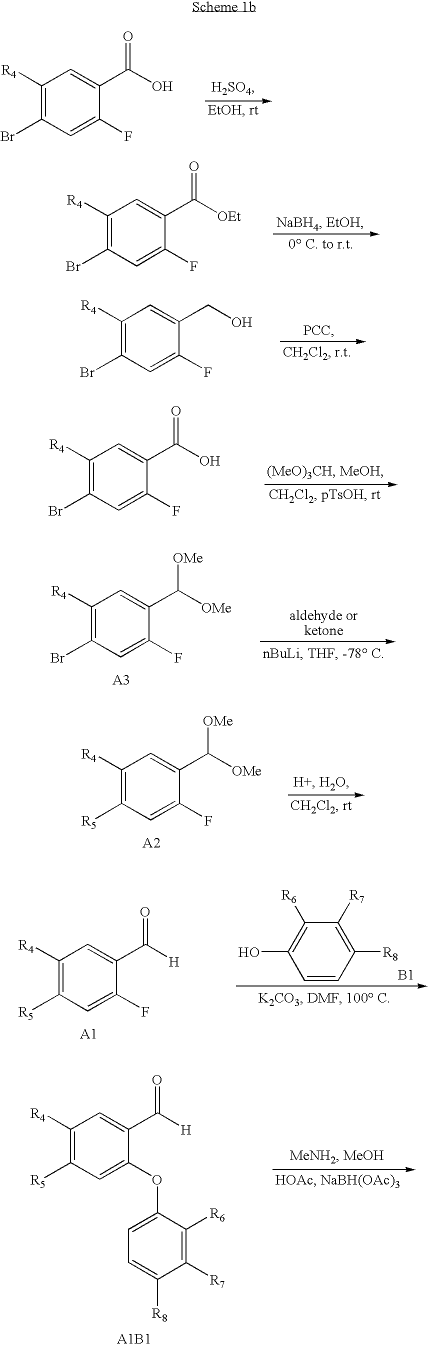

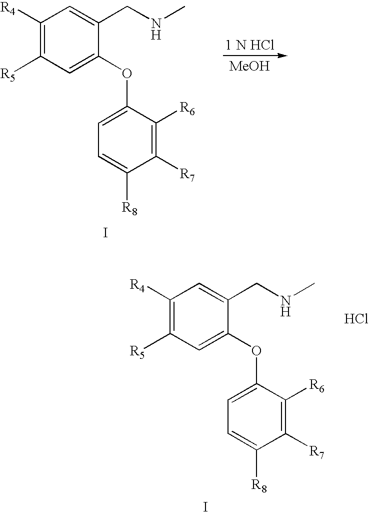

The variables used in Scheme 1b are as defined herein. In step 5, the arylbromide intermediate A3 can be converted into an organometallic reagent such as an aryl lithium or an aryl Grignard by treatment with an alkyl lithium reagent or a Grignard reagent respectively. The resulting aryl anion can be trapped with an electrophile such as and aldehyde or ketone to generate substituents at R5 that contain a functional group such as an alcohol. The material generated in step 5 can be carried on directly to compounds of formula I. Alternatively the material generate in step 5 can be alkylated with a strong base, such as lithium diisopropylamide (LDA), sodium hydride, or a similar reagent and an alkylating reagent such as methyl iodide or ethyl iodide to introduce O-alkoxy moieties for R¹¹ or R¹² which in turn can be converted to compounds of formula I.

Compounds of general formula I can also be prepared according to the procedure shown in Scheme 2. Referring to Scheme 2, an A-ring containing a carboxylic acid can readily be converted to an amide by a wide variety of amide coupling methods known to those skilled in the art, but preferable using an amine in the presence of a carbodiimide reagent such as CDI. The resulting fluoroamide can be reacted with a phenol B-ring in the presence of a base, preferable sodium tert-butoxide in an inert reaction solvent such as THF or 2-methyl THF to give the analogous A1B1 amide product. The amide is then reduced to the desired amino compound in the presence of a reducing agent in an inert reaction solvent, preferably sodium borohydride in Me-THF.

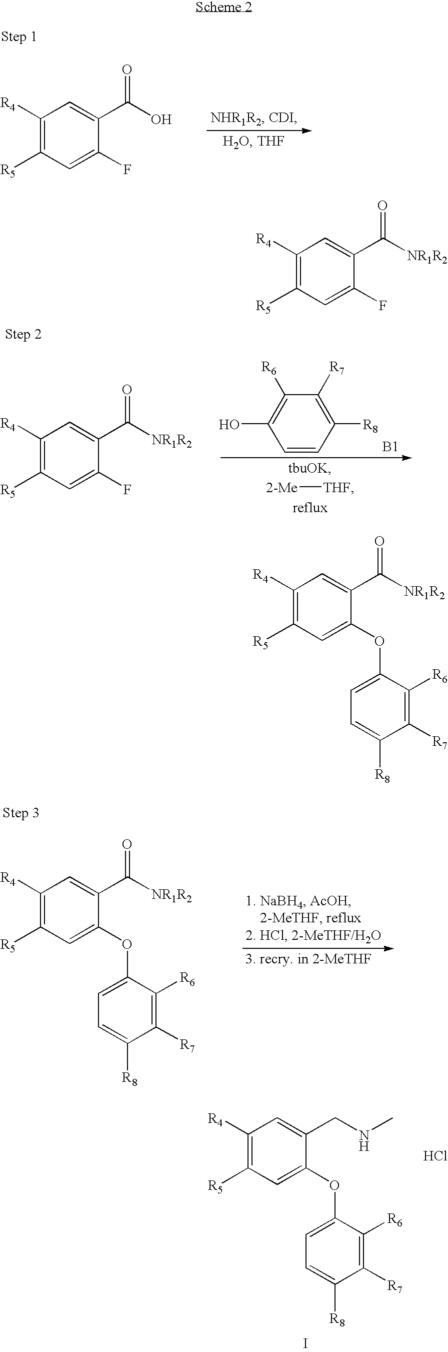

Compounds of general formula I can also be prepared according to the Scheme 3 shown below. In particular a compound of formula A1B1-1 can be reacted with an organothiolate to give an aryl thio-ether compound of formula A1B1-2. After installation of the amino group and protection, the thioether can be oxidized to a sulfoxide or a sulfone in the presence of an oxidizing agent, preferable mCPBA or hydrogen peroxide. Removal of the amine protecting group provides compounds of formula I where R5 is a sulfoxide or a sulfone. 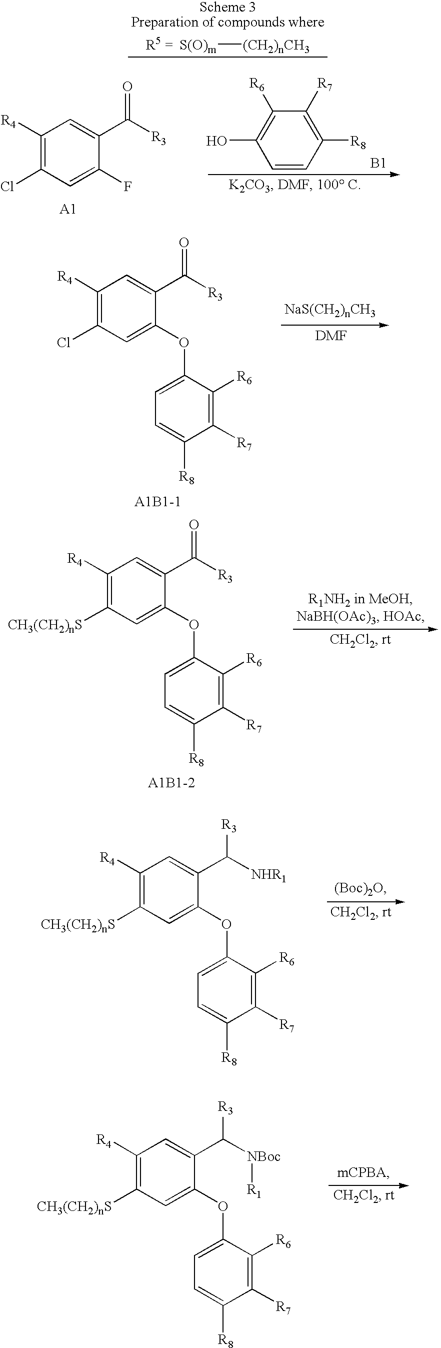 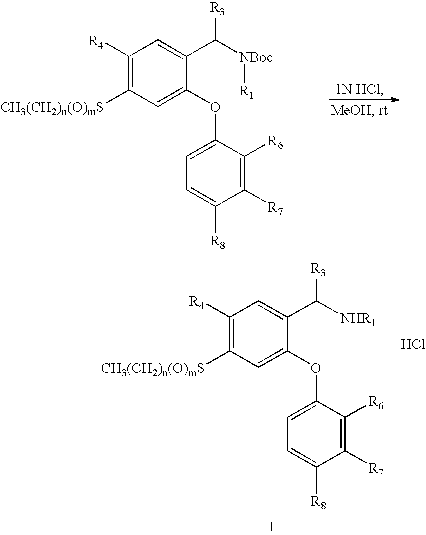
Compounds of general formula I can also be prepared according Scheme 4. Referring to Scheme 4 a compound of formula I where R═CR¹¹R¹²—(CH₂)ₙCH₃, and more specifically where R¹¹ or R¹² is hydroxyl can be prepared by reacting a compound of formula A1B1 where R5=CHO with a Grignard reagent followed by installation of the amino group (NR1R2) via the methods described previously. A compound of formula A1B1 where R5=CHO can be prepared by reacting a compound of formula A1B1 where R═Br with an organolithium reagent followed by DMF.

Compounds of Formula Ib can be prepared according to Scheme 5 which describes the removal of a protecting group after the reductive amination process (a similar process is described in Scheme 3).

Compounds of Formula I where R⁵═C₁-C₆ alkyl, C₁-C₆ branched alkyl or cycloalkyl can be prepared according to the method of Hayashi, T., et al. J. Amer. Chem. Soc. 1984, 106, 158. An example is shown in Scheme 6.

In some cases where the B-group in Formula I contains a heterocycle, the heterocyclic portion of the molecule is installed after the diarylether coupling step. Routes exemplifying, but not limiting, this are shown in the Schemes 7-10. 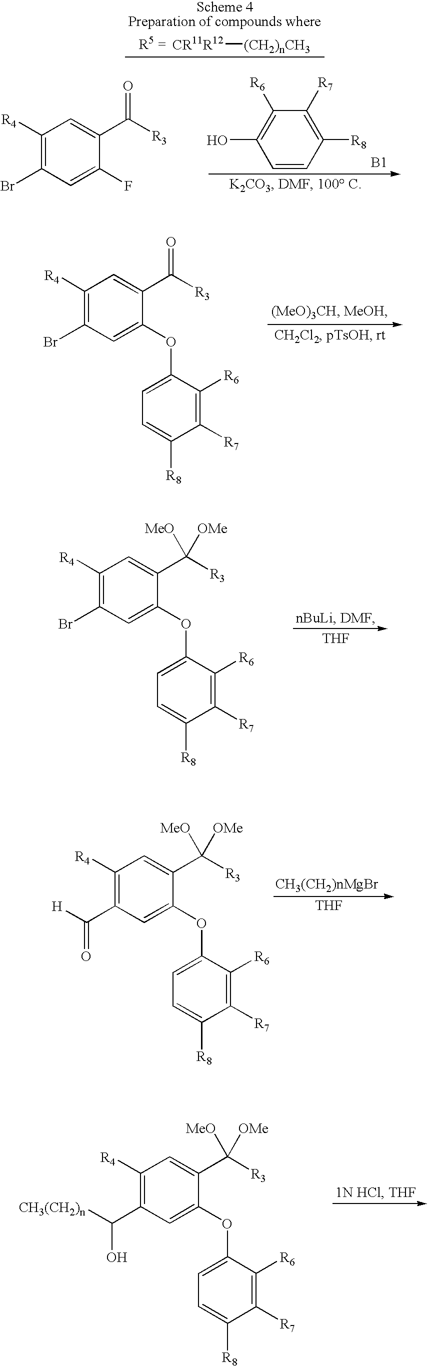 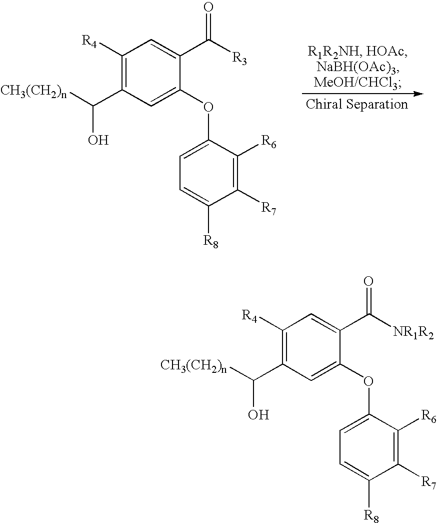 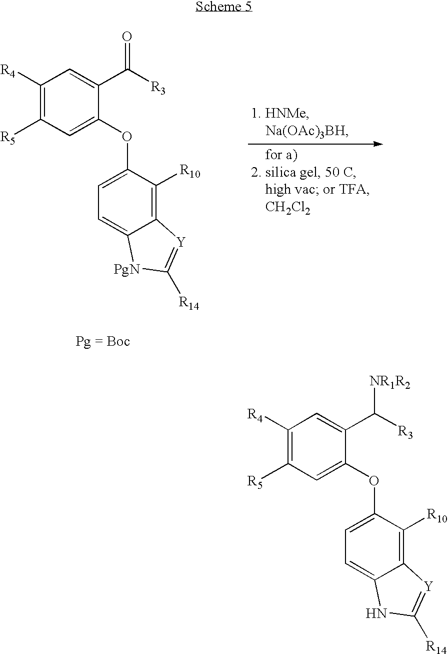 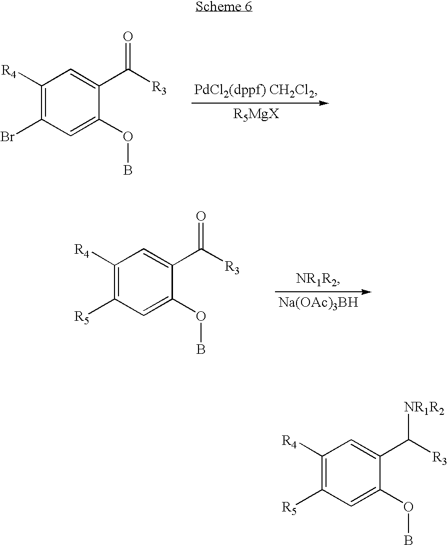
Referring to Scheme 7, the carbonyl functional group in the compound of formula A1B1 was protected and the resulting product was subjected to Bartoli indole synthesis (see Bartoli, G., et. al. Tetrahedron Lett., 1989, 30, 2129). The resulting product where the B-ring contains an indole can be taken directly to a compound of formula Ic where the indole nitrogen is unsubstituted. Alternatively, the indole nitrogen may be alkylated prior to liberation of the carbonyl functional group to ultimately provide a compound of formula Ic where the indole nitrogen has an alkyl group (R18) attached. 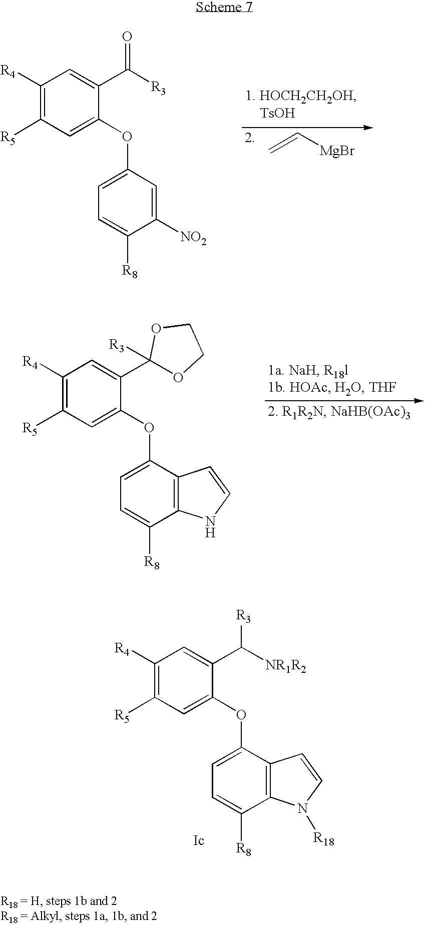
Referring to Scheme 8, the benzoxazolinone functionality was introduced into the B-ring after introducing an appropriate protection group on the amine functional group on the A-ring, preferably trifluoroacetyl in this instance. Simultaneous removal of the benzyl group on the phenol and reduction of the nitro aromatic group provided an intermediate amino phenol group that was reacted with a carbonyl equivalent, preferably CDI to provide the benzoxazolinone group. Final preparation of the compound of formula Ib resulted from the removal of the protecting group.

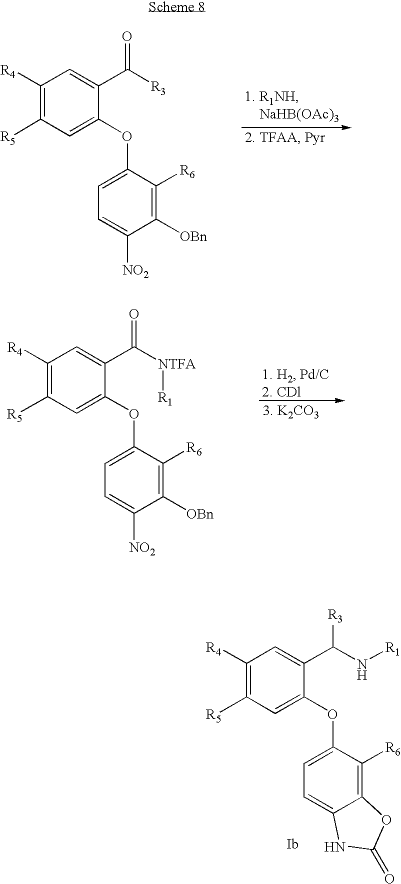

Referring to Scheme 9 and Scheme 10, the preparation of a compound of formula Ib where the B-ring portion of the molecule is benzoxazinone or dihydrobenzoxazine is described. The benzoxazinone phenol was prepared through the reaction of the A-ring precursor shown below with alpha bromomethyl acetate followed by reduction and cyclization. The benzoxazinone may be alkylated on the nitrogen prior to making the phenol by the treatment with an alkylating reagent and a base, preferably sodium hydride. Removal of the benzyl protecting group followed by standard coupling and amine installation as described previously will provide compounds of formula Ib where the B-ring is a benzoxazinone. The corresponding compounds of formula Ib where the B-ring is benzoxazine can be prepared by treating the benzoxazinone compounds with a reducing agent, preferable borane.

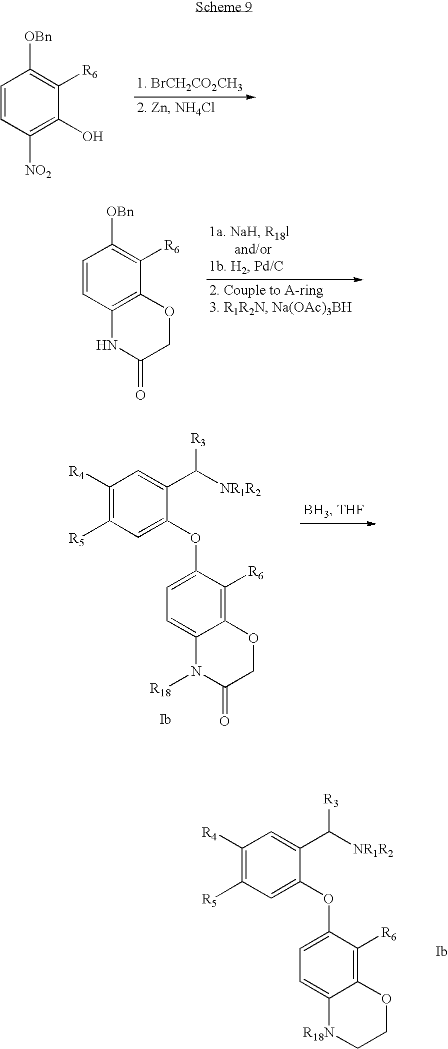

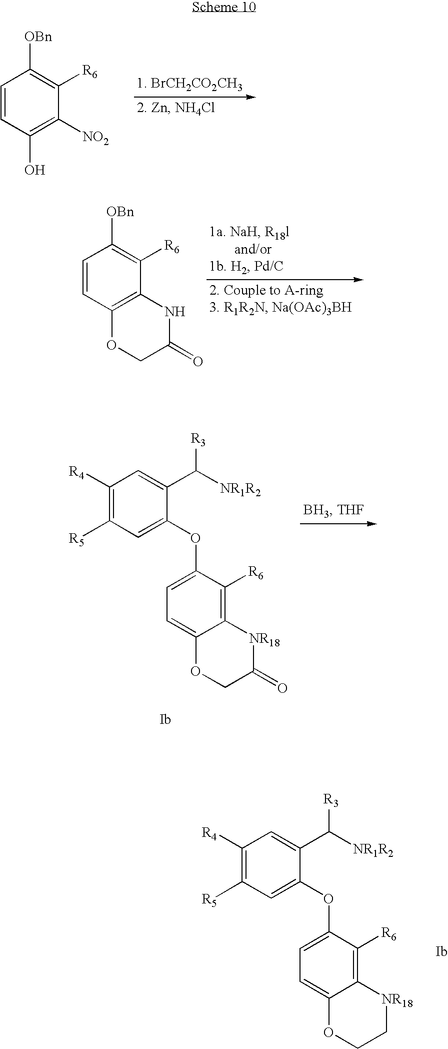

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of this invention are useful also in the diagnostic imaging of tissue in patients afflicted with or suspected of being afflicted with diseases or disorders of the central or peripheral nervous system. Imaging of tissue can be achieved by means of conventional diagnostic in vivo imaging protocols which are well known in the art. To summarize, a substance which is capable of detection within a patient, i.e., a labeled substance such as a radionuclide-labeled (“radiolabeled”) receptor agonist or antagonist, is administered to a patient in an amount sufficient to deliver an adequate supply of labeled substance to the target tissue so as to permit an image to be generated. The radionuclide provides the imaging input, with emission of a particle characteristic of radioactive decay, such as a gamma ray. Detection of the particle then permits imaging of the tissue or organ while the labeled substance is bound to that tissue or organ.

Radiolabeled compounds of Formula I can be prepared by incorporation into the synthetic procedures described herein of techniques of isotopic labeling that are well known in the art. Any radioisotope capable of being detected can be employed as a label. A compound is radiolabeled either by substitution of a radioactive isotope of hydrogen, carbon, or fluorine or by incorporation of a phenyl group that is substituted with radioactive iodine. Suitable radioisotopes include carbon-11, fluorine-18, fluorine-19, iodine-123 and iodine-125. For example, see Arthur Murry III, and D. Lloyd Williams, “Organic Synthesis with Isotopes,” vols. I and II, Interscience Publishers Inc., N.Y. (1958) and Melvin Calvin et al. “Isotopic Carbon,” John Wiley and Sons Inc., N.Y. (1949). Preferably, a compound of Formula I may be labeled by adding one or more radioisotopes of a halogen (e.g. iodine-123) to an aromatic ring, or by alkylating a nitrogen atom in a compound of Formula I with a group comprising a phenyl group bearing a radioisotope.

The compounds of the formula Ia, Ib or Ic and their pharmaceutically acceptable salts (hereafter “the active compounds”) can be administered via either the oral, transdermal (e.g., through the use of a patch), intranasal, sublingual, rectal, parenteral or topical routes. Transdermal and oral administration are preferred. These compounds are, most desirably, administered in dosages ranging from about 0.25 mg up to about 1500 mg per day, preferably from about 0.25 to about 300 mg per day in single or divided doses, although variations will necessarily occur depending upon the weight and condition of the subject being treated and the particular route of administration chosen. However, a dosage level that is in the range of about 0.01 mg to about 10 mg per kg of body weight per day is most desirably employed. Variations may nevertheless occur depending upon the weight and condition of the persons being treated and their individual responses to said medicament, as well as on the type of pharmaceutical formulation chosen and the time period and interval during which such administration is carried out. In some instances, dosage levels below the lower limit of the aforesaid range may be more than adequate, while in other cases still larger doses may be employed without causing any harmful side effects, provided that such larger doses are first divided into several small doses for administration throughout the day.

The active compounds can be administered alone or in combination with pharmaceutically acceptable carriers or diluents by any of the several routes previously indicated. More particularly, the active compounds can be administered in a wide variety of different dosage forms, e.g., they may be combined with various pharmaceutically acceptable inert carriers in the form of tablets, capsules, transdermal patches, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents. In addition, oral pharmaceutical compositions can be suitably sweetened and/or flavored. In general, the active compounds are present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc can be used for tableting purposes. Solid compositions of a similar type may also be employed as fillers in gelatin capsules; preferred materials in this connection also include lactose or milk sugar] as well as high molecular weight polyethylene glycols. When aqueous suspensions and/or elixirs are desired for oral administration the active ingredient may be combined with various sweetening or flavoring agents, coloring matter and, if so desired, emulsifying and/or suspending agents, together with such diluents as water, ethanol, propylene glycol, glycerin and various combinations thereof.

For parenteral administration, a solution of an active compound in either sesame or peanut oil or in aqueous propylene glycol can be employed. The aqueous solutions should be suitably buffered (preferably pH greater than 8), if necessary, and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art.

It is also possible to administer the active compounds topically and this can be done by way of creams, a patch, jellies, gels, pastes, ointments and the like, in accordance with standard pharmaceutical practice.

The activity of the compounds of the present invention with respect to 5HT₂ receptor binding ability can be determined according to the protocol of Pazos, A., et al., Eur. J. Pharm., 1984, 106, 539-546. Affinities for 5HT_{2A} receptor were determined (K_{i's}) for the compounds, and are in all cases less than 100 nM.

The following Examples were prepared using the preparative methods described above. Specific Examples were prepared using the General Procedure or any one of the schemes described above:

General Procedure: A mixture of A1 (fluorobenzaldehyde, 1 mmol), B1 (phenol, 0.5-2 mmol), and base (K₂CO₃ or Cs₂CO₃ preferred, 1-5 mmol) are combined in solvent (DMF or THF preferred, 0.1-1.0 M) and heated (80-110° C.) for 10-20 h. The reaction mixture is cooled and partitioned between organic solvent (EtOAc or CH₂Cl₂) and H₂O. The layers are separated and the aqueous layer is extracted with organic solvent. The organic extracts are combined, washed with 1 N NaOH, 1 N LiCl, and H₂O, dried (MgSO₄), filtered, and concentrated. The crude material is chromatographed on silica gel using EtOAc/Hexane mixtures to provide the desired coupled product. The product (1 mmol) is then dissolved in solvent (CH₂Cl₂, MeOH or CH₃CN preferred, 0.05-2 M) and methylamine (soln in MeOH or THF, 2-5 mmol) is added. Other reagents that are optionally added include acetic acid, Na₂SO₄, and 4 angstrom molecular sieves. The mixture was stirred for 1-20 h at rt. The reducing agent (NaBH(OAc)₃, NaBH₃CN, or NaBH₄ preferred, 1-5 mmol) was added and the reaction mixture continued stirring at rt for 10-24 h. The reaction mixture was diluted with aqueous solution (sat. NaHCO₃ or 1M NaOH) and extracted (EtOAc preferred). The combined extracts were washed with H₂O, dried (MgSO₄), filtered, concentrated and chromatographed (eluted with MeOH/CH₂Cl₂) to provide the desired example.

### EXAMPLE 1

### 1-[3-(2-Chloro-4-fluoro-3-methylphenoxy)-4-methylaminomethylphenyl]propan-1-ol

MS (M⁺)=338, 340.

### EXAMPLE 2

### (S)-1-[2-Fluoro-5-(7-fluoroindan-4-yloxy)-4-methylaminomethylphenyl]propan-1-ol

MS (M⁺)=348, 349

### EXAMPLE 3

### [4-Bromo-2-(3-methoxy-2-methylphenoxy)benzyl]methylamine

MS (M⁺)=336, 338.

### EXAMPLE 4

### 2-[3-(3-Methoxy-2-methylphenoxy)-4-methylaminomethylphenyl]propan-2-ol

MS (M⁺)=316.

### EXAMPLE 5

### [4-Chloro-2-(3-methoxy-2-methylphenoxy)-5-methylbenzyl]methylamine

MS (M⁺)=306, 308.

### EXAMPLE 6

### [2-(4-Chlorophenoxy)-4-methanesulfonylbenzyl]methylamine

MS (M⁺)=326, 328.

### EXAMPLE 7

### 1-[3-(4-Chloro-3-methoxy-2-methylphenoxy)-4-methylaminomethylphenyl]propan-1-ol

MS (M⁺)=350, 352.

### EXAMPLE 8

### [4-Chloro-2-(4-chloro-3-methoxy-2-methylphenoxy)-5-fluorobenzyl]methylamine

MS (M⁺)=344, 346.

### EXAMPLE 9

### [4-Bromo-2-(4-chloro-3-methoxy-2-methylphenoxy)benzyl]methylamine

### EXAMPLE 10

### 6-Chloro-3-(5-chloro-4-fluoro-2-methylaminomethylphenoxy)-2-methylphenol

MS (M⁺)=330, 332.

### EXAMPLE 11

### [4-Chloro-2-(3-methoxy-2-methylphenoxy)benzyl]methylamine

MS (M⁺)=292, 294.

### EXAMPLE 12

### [4-Bromo-2-(3-methoxy-2-methylphenoxy)-5-methylbenzyl]methylamine

MS (M⁺)=350, 352.

### EXAMPLE 13

### 1-[3-(4-Chloro-2,3-dimethylphenoxy)-4-methylaminomethylphenyl]propan-1-ol

4-Bromo-2-fluoro-benzaldehyde (150 g, 735.94 mmol), 4-chloro-2,3-dimethylphenol (126.26 g, 806.21 mmol), and K₂CO₃ (305.10 g, 2.21 mole) were combined in acetonitrile (1.3 L) and heated at reflux for 21 h. The solution was cooled, concentrated to ½ volume, diluted with H₂O (2.0 L), and extracted with EtOAc (3×900 mL). The combined organic extracts were washed with 1N NaOH (1.0 L), H₂O (3×1.0 L), dried (MgSO₄), filtered, and concentrated to provide 249.94 g of 4-bromo-2-(4-chloro-2,3-dimethylphenoxy)benzaldehyde as a light golden solid.

4-Bromo-2-(4-chloro-2,3-dimethylphenoxy)benzaldehyde (249.94 g, 735.94 mmol) and p-toluenesulfonic acid monohydrate (14.0 g, 73.59 mmol) were combined in MeOH (4.0 L) and heated at reflux for 15 h. The solution was cooled, diluted with 1N Na₂CO₃ (1.0 L) and evaporated to remove the MeOH. Mix was diluted with H₂O (500 mL) and extracted with EtOAc (3×800 mL). The combined organic extracts were washed with H₂O (2×1.0 L), dried (Na₂SO₄), filtered, and concentrated to provide 281.41 g of 1-[5-bromo-2-(dimethoxymethyl)phenoxy]-4-chloro-2,3-dimethylbenzene as a golden oil.

1-[5-Bromo-2-(dimethoxymethyl)phenoxy]-4-chloro-2,3-dimethylbenzene (75.0 g, 194.46 mmol) was dissolved in THF (450 mL) and cooled to −78° C. n-Butyl lithium (101.1 mL of a 2.5 M solution in hexanes) was rapidly added and immediately followed by addition of Propionaldehyde (21.23 mL, 291.69 mmol). Reaction was stirred in the cold for 30 min then allowed to warm to r.t. and stirred for 2 h. The reaction solution was diluted with H₂O (1.5 L) and extracted with EtOAc (3×700 mL). The combined organic extracts were washed with H₂O (2×1.0 L), dried (Na₂SO₄), filtered, concentrated, and chromatographed (eluted with 10% EtOAc in hexanes) to provide 56.88 g of 1-[3-(4-chloro-2,3-dimethylphenoxy)-4-(dimethoxymethyl)phenyl]propan-1-ol as a golden oil.

1-[3-(4-Chloro-2,3-dimethylphenoxy)-4-(dimethoxymethyl)phenyl]propan-1-ol (56.80 g, 155.67 mmol) was dissolved in THF (420 mL) and 1N HCl (155.67 mL, 155.67 mmol) was added and the solution was stirred for 15 h. The reaction was diluted with H₂O (1.0 L) and extracted with EtOAc (3×500 mL). The combined organic extracts were washed with saturated NaHCO₃ (300 mL), H₂O (2×800 mL), dried (MgSO₄), filtered, and concentrated to provide 49.63 g of 2-(4-chloro-2,3-dimethylphenoxy)-4-(1-hydroxypropyl)benzaldehyde as a golden oil.

2-(4-chloro-2,3-dimethylphenoxy)-4-(1-hydroxypropyl)benzaldehyde (49.63 g, 155.67 mmol), methylamine (155.7 mL of a 2.0M solution in MeOH, 311.34 mmol), and acetic acid (26.73 mL, 467.01 mmol) were combined in CH₂Cl₂ and stirred for 8 h. NaBH(OAc)₃ was added and reaction was stirred for 15 h. Reaction mix was diluted with saturated NaHCO₃ until a pH of 7 was obtained and extracted with EtOAc (3×600 mL). The combined organic extracts were washed with brine (1.0 L), dried (MgSO₄), filtered, concentrated, added EtOAc (500 mL) and filtered 27.6 g of 1-[3-(4-chloro-2,3-dimethylphenoxy)-4-methylaminomethylphenyl]propan-1-ol as a pale yellow crystalline solid. MS (M⁺) 334. Enantiomers were separated using chiral HPLC (Chiralpak AD column; 92/8 heptane/EtOH mobile phase with 0.2% diethylamine modifier). Isolated oil was dissolved in 1N HCl in MeOH, evaporated, azetroped with 2× Et₂O, added EtOAc to yield off-white crystalline solid. MS (M⁺)=334, 336. Absolute stereochemistry of the S-enantiomer was proven by X-Ray Crystallography as the HCl salt.

### EXAMPLE 14

### [4-Chloro-2-(4-chloro-2,3-dimethylphenoxy)-5-fluorobenzyl]methylamine

MS (M⁺)=328, 330.

### EXAMPLE 15

### [2-(4-Chloro-2,3-dimethylphenoxy)-4-methanesulfonylbenzyl]methylamine

MS (M⁺)=354.

### EXAMPLE 16

### [4-Chloro-2-(4-chloro-2,3-dimethylphenoxy)-5-methylbenzyl]methylamine

MS (M⁺)=324, 326.

### EXAMPLE 17

### [4-Bromo-2-(4-chloro-2,3-dimethylphenoxy)benzyl]methylamine

MS (M⁺)=354, 356, 358.

### EXAMPLE 18

### [2-(4-Chloro-2,3-dimethylphenoxy)-4-methanesulfonyl-5-methylbenzyl]methylamine

MS (M⁺)=368, 370.

### EXAMPLE 19

### [2-(4-Chloro-2,3-dimethylphenoxy)-4-methanesulfinyl-5-methylbenzyl]methylamine

Melting point=220-222° C.

### EXAMPLE 20

### [4-Chloro-2-(4-chloro-2,3-dimethylphenoxy)benzyl]methylamine

MS (M⁺)=310, 312.

### EXAMPLE 21

### 1-[3-(4-Chloro-2,3-dimethylphenoxy)-4-methylaminomethylphenyl]ethanol

MS (M⁺)=320, 322.

### EXAMPLE 22

### [3-(4-Chloro-2,3-dimethylphenoxy)-4-methylaminomethylphenyl]methanol

MS (M⁺)=306, 308.

### EXAMPLE 23

### [2-(4-Chloro-2,3-dimethylphenoxy)-4-(pyrrolidine-1-sulfonyl)benzyl]methylamine

MS (M⁺)=408.

### EXAMPLE 24

### [2-(4-Chloro-2,3-dimethylphenoxy)-4-methoxymethylbenzyl]methylamine

MS (M⁺)=320, 322.

### EXAMPLE 25

### [2-(4-Chloro-2,3-dimethylphenoxy)-4-(1-methoxypropyl)benzyl]methylamine

### EXAMPLE 26

### [2-(4-Chloro-2,3-dimethylphenoxy)-4-(1-methoxyethyl)benzyl]methylamine

### EXAMPLE 27

### 1-[3-(4-Chloro-2,3-dimethylphenoxy)-4-methylaminomethylphenyl]prop-2-yn-1-ol

MS (M⁺)=330.

### EXAMPLE 28

### 1-[3-(4-Chloro-2,3-dimethylphenoxy)-4-methylaminomethylphenyl]-pent-4-en-1-ol

MS (M⁺)=360.

### EXAMPLE 29

### 1-[3-(4-Chloro-2,3-dimethylphenoxy)-4-methylaminomethylphenyl]-3-phenyl-prop-2-yn-1-ol

MS (M⁺)=406.

### EXAMPLE 30

### [3-(4-Chloro-2,3-dimethylphenoxy)-4-methylaminomethylphenyl]-(2-methoxy-phenyl)-methanol

MS (M⁺)=412.

### EXAMPLE 31

### 1-[3-(4-Chloro-2,3-dimethylphenoxy)-4-methylaminomethylphenyl]-2-methyl-prop-2-en-1-ol

MS (M⁺)=346.

### EXAMPLE 32

### 1-[3-(4-Chloro-2,3-dimethylphenoxy)-4-methylaminomethylphenyl]-but-3-en-1-ol

MS (M⁺)=346.

### EXAMPLE 33

### [3-(4-Chloro-2,3-dimethylphenoxy)-4-methylaminomethylphenyl]-(3-fluorophenyl)-methanol

MS (M⁺)=400.

### EXAMPLE 34

### 1-[3-(3-Methoxy-2-methylphenoxy)-4-methylaminomethylphenyl]propan-1-ol

### EXAMPLE 35

### [2-(3-Methoxy-2-methylphenoxy)-4-(1-methoxypropyl)benzyl]methylamine

MS (M⁺)=330.

### EXAMPLE 36

### [2-(4-Chloro-3-methoxy-2-methylphenoxy)-4-(1-methoxypropyl)benzyl]methylamine

MS (M⁺)=364, 366.

### EXAMPLE 37

### [4-Chloro-2-(4-chloro-2-fluorophenoxy)benzyl]methylamine

MS (M⁺)=300, 302.

### EXAMPLE 38

### [4-Chloro-2-(4-chloro-2-fluorophenoxy)-5-fluorobenzyl]methylamine

MS (M⁺)=318, 320.

### EXAMPLE 39

### [4-Chloro-2-(4-chloro-2-fluorophenoxy)-5-fluorobenzyl]-dimethylamine

MS (M⁺)=332, 334.

### EXAMPLE 40

### [2-(4-Chloro-2-fluorophenoxy)-5-fluoro-4-methylbenzyl]methylamine

MS (M⁺)=298.

### EXAMPLE 41

### {1-[4-Chloro-2-(4-chloro-2-fluorophenoxy)-5-fluorophenyl]ethyl}-methylamine

MS (M⁺)=332, 334. Melting point=178-180° C.

### EXAMPLE 42

### [1-[4-Chloro-2-(4-chloro-2-fluorophenoxy)-5-fluorophenyl]ethyl}-dimethylamine

### EXAMPLE 43

### [4-Chloro-2-(4-chloro-2-fluorophenoxy)-5-methylbenzyl]methylamine

MS (M⁺)=314, 316. Melting point=212-214° C.

### EXAMPLE 44

### [5-Chloro-2-(4-chloro-2-fluorophenoxy)-4-methylbenzyl]methylamine

MS (M⁺)=314, 316.

### EXAMPLE 45

### [2-(4-Chloro-2-fluorophenoxy)-4,5-dimethylbenzyl]methylamine

Melting point=134-136° C.

### EXAMPLE 46

### [2-(4-Chloro-2-fluorophenoxy)-4-methoxy-benzyl]methylamine

Melting point=186-188° C.

### EXAMPLE 47

### [2-(4-Chloro-2-fluorophenoxy)-4-methylbenzyl]methylamine

Melting point=198-20° C.

### EXAMPLE 48

### [2-(4-Chloro-2-fluorophenoxy)-5-fluoro-4-propylsulfanylbenzyl]methylamine

MS (M⁺)=358, 360.

### EXAMPLE 49

### [2-(4-Chloro-2-fluorophenoxy)-5-fluoro-4-isopropylsulfanylbenzyl]methylamine

MS (M⁺)=358, 360.

### EXAMPLE 50

### [4-Bromo-2-(4-chloro-2-fluorophenoxy)benzyl]methylamine

Melting point=204-206° C.

### EXAMPLE 51

### [4-Chloro-2-(4-chloro-2-fluoro-3-methylphenoxy)-5-fluorobenzyl]methylamine

MS (M⁺)=332.

### EXAMPLE 52

### [2-(4-Chloro-2-fluorophenoxy)-5-fluoro-4-methanesulfonylbenzyl]methylamine

Melting point=184-186° C.

### EXAMPLE 53

### [4-(Butane-1-sulfonyl)-2-(4-chloro-2-fluorophenoxy)-5-fluorobenzyl]methylamine

MS (M⁺)=404, 406.

### EXAMPLE 54

### [2-(4-Chloro-2-fluorophenoxy)-5-fluoro-4-(propane-1-sulfonyl)benzyl]methylamine

MS (M⁺)=390.

### EXAMPLE 55

### [2-(4-Chloro-2-fluoro-3-methylphenoxy)-4-methanesulfonylbenzyl]methylamine

MS (M⁺)=358.

### EXAMPLE 56

### 1-[3-(4-Chloro-2-fluorophenoxy)-4-methylaminomethylphenyl]propan-1-ol

MS (M⁺)=324, 326.

### EXAMPLE 57

### [4-Chloro-2-(7-fluoroindan-4-yloxy)benzyl]methylamine

MS (M⁺)=306, 308. Melting point=204-206° C.

### EXAMPLE 58

### [2-(Indan-5-yloxy)-4-methanesulfonylbenzyl]methylamine

Melting point=88-90° C.

### EXAMPLE 59

### [4-Methanesulfonyl-2-(naphthalen-2-yloxy)benzyl]methylamine

MS (M⁺)=342.

### EXAMPLE 60

### [2-(4-Chlorophenoxy)-4-ethylsulfanylbenzyl]methylamine

Melting point=158-160° C.

### EXAMPLE 61

### 4-(5-Chloro-4-fluoro-2-methylaminomethylphenoxy)-2-methylphenol

MS (M⁺)=296, 298.

### EXAMPLE 62

### [4-Chloro-5-fluoro-2-(3-methoxy-2-methylphenoxy)benzyl]methylamine hydrochloride salt

Preparation 1 benzaldhyde (15.33 g, 86.85 mmol), 3-methoxy-2-methylphenol (12.0 g, 86.85 mmol) and powdered K₂CO₃ (36.01 g, 260.55 mmol) were combined in DMF (150 mL) and heated at 100° C. for 15 h. The mixture was cooled, diluted with H₂O (800 mL) and extracted with EtOAc (3×400 mL). The combined organic extracts were washed with 1N NaOH (2×300 mL), 1N LiCl (2×400 mL), H₂O (600 mL), dried (MgSO₄), filtered, concentrated and chromatographed (preabsorbed onto silica gel; eluted with 5% EtOAc in hexanes) to provide 16.50 g of 4-chloro-5-fluoro-2-(3-methoxy-2-methylphenoxy)benzaldehyde as a pale yellow solid. 4-Chloro-5-fluoro-2-(3-methoxy-2-methylphenoxy)benzaldehyde (16.4 g, 55.65 mmol), monomethyl amine (55.7 mL of a 2.0 M solution in MeOH, 111.30 mmol), acetic acid (9.56 mL, 166.95 mmol) were combined in CH₂Cl₂ (50 mL) and stirred for 3 h. To the reaction mixture was added NaBH(OAc)₃ (17.69 g, 83.48 mmol) and stirred for 15 h. The mixture was diluted with saturated NaHCO₃ (800 mL) and extracted with EtOAc (3×400 mL). The combined organic extracts were washed with H₂O (1 L), dried (MgSO₄), filtered, concentrated, chromatographed (eluted with 10% MeOH in CHCl₃), dissolved in Et₂O (200 mL), added 1N HCl in Et₂O (100 mL), concentrated, azetroped with Et₂O (2×100 mL), added Et₂O (100 mL) and filtered to provide 10.56 g of [4-chloro-5-fluoro-2-(3-methoxy-2-methylphenoxy)benzyl]methylamine hydrochloride salt as a white solid. MS (M⁺)=310, 312. ¹H NMR (400 MHz, CDCl₃) δ 9.92 (m, NH, HCl), 7.68 (d, 1, J=8.7), 7.13 (t, J=8.3 Hz, 1H), 6.70 (d, J=8.3 Hz, 1H), 6.59 (m, 2H), 4.25 (t, J=5.4 Hz, 2H), 3.83 (s, 3H), 2.65 (t, J=5.8 Hz, 3H), 2.01 (s, 3H). ¹³C NMR (100 MHz, CDCl₃): δ 159.34, 154.69, 153.60, 153.11, 152.25, 127.56, 123.56, 123.37, 119.94, 119.70, 119.33, 119.27, 118.86, 117.70, 112.705, 112.50, 107.45, 55.98, 45.34, 31.78, 9.14. Analysis calculated for C₁₆H₁₇ClFNO₂.HCl: C, 55.51; H, 5.24; N, 4.05; Cl, 20.48; F, 5.49. Found: C, 55.44; H, 5.17; N, 3.99; Cl, 20.64; F, 5.62.

### EXAMPLE 63

### [4-Chloro-2-(1,3-dihydro-isobenzofuran-5-yloxy)benzyl]methylamine formic acid salt

To a solution of 4-chloro-2-fluorobenzaldehyde (1 mmol) in DMF (8 mL) was added Preparation 8 (1.2 mmol) and K₂CO₃ (3 mmol). The reaction mixture was heated at 100° C. for 6 h, cooled, diluted with EtOAc, filtered through a plug of Celite™, and concentrated. The resulting residue was dissolved in 1,2-dichloroethane (6 mL) and to this solution was added methyl amine (2 mmol, 1 mL of a 2 M solution in methanol) and glacial acetic acid (3 mmol). After stirring overnight at rt, NaBH(OAc)₃ (1.5 mmol) was added. The reaction mixture was stirred for 24 h, diluted with 2 M NaOH (20 mL), and extracted with CH₂Cl₂ (5×20 mL). The combined extracts were dried (MgSO₄), filtered and concentrated. The residue was dissolved in DMSO and purified by reverse-phase HPLC (19×100 mm Exterra column; 8 min gradient; 25 mL/min; 15-100% acetonitrile in water with 0.1% formic acid modifier) to provide the above named compound as a formic acid salt. MS (M⁺)=290.3 292.3. ¹H NMR (400 MHz, CD₃OD): δ 8.45 (s, 1), 7.52 (d,1, J=8.3), 7.36 (d, 1, J=7.9), 7.18 (d, 1, J=2), 7.16 (d, 2, J=2), 6.76 (d, 1, J=2), 5.07 (s, 4), 4.95 (s, 3), 2.75 (s, 2).

### EXAMPLE 64

### [4-Chloro-2-(2,3-dihydrobenzo[b]thiophen-5-yloxy)benzyl]methylamine formic acid salt

Prepared according to the procedure of Example 62 except using Preparation 5 phenol. MS (M⁺)=306.3, 308.3. ¹H NMR (400 MHz, CD₃OD): δ 8.43 (s, 1), 7.49 (d, 1, J=8.3), 7.23 (d, 1, J=8.3), 7.13 (d, 1, J=2), 7.03 (t, 1, 1.24), 6.9 (m, 1), 6.73 (d, 1, J=2H), 4.29 (s, 1), 3.4 (m, 2), 3.3 (m, 2), 2.74 (s, 3).

### EXAMPLE 65

### [4-Chloro-2-(2,3-dihydrobenzo[1,4]oxathiin-7-yloxy)benzyl]methylamine formic acid salt

Prepared according to the procedure of Example 62 except using Preparation 9 phenol. MS (M⁺)=322.3, 324.3. ¹H NMR (400 MHz, CD₃OD): δ 8.41 (s, 1), 7.49 (d, 1, J=8.29), 7.17 (d, 1, J=2), 7.09 (d, 1, J=8.3), 6.79 9 (s, 1), 6.6 (m, 2), 4.4 (m, 2), 4.26 (s, 2), 3.16 (m, 2), 2.73 (s, 3).

### EXAMPLE 66

### [4-Chloro-2-(2,3-dihydrobenzo[b]thiophen-6-yloxy)benzyl]methylamine formic acid salt

Prepared according to the procedure of Example 62 except using Preparation 6 phenol. MS (M⁺)=306.3, 308.3. ¹H NMR (400 MHz, CDCl₃): δ 8.5 (s, 1), 7.48 (d, 1, J=8.3), 7.25 9 (m, 1), 7.15 (m, 1), 6.97 (s, 1), 6.75 (m, 2), 4.25 (s, 2), 3.41 (m, 2), 3.39 (m, 2), 2.73 (s, 3).

### EXAMPLE 67

### [4-Chloro-2-(2,3-dihydrobenzo[1,4]oxathiin-6-yloxy)benzyl]methylamine formic acid salt

Prepared according to the procedure of Example 62 except using Preparation 10 phenol. MS (M⁺)=322.3, 324.3. ¹H NMR (400 MHz, CDCl₃): δ 8.4 (s, 1), 4.46 (d, 1, J=8.3), 7.13 (m, 1), 6.86 (m, 2), 6.79 (m, 1), 6.75 (m, 1), 4.4 (m, 2), 4.27 (s, 2), 3.3 (m, 2), 2.73 (s, 3).

### EXAMPLE 68

### [4-Chloro-2-(1,3-dihydrobenzo[c]thiophen-5-yloxy)benzyl]methylamine formic acid salt

Prepared according to the procedure of Example 62 except using Preparation 7 phenol. MS (M⁺)=306.3, 308.3. ¹H NMR (400 MHz, CDCl₃): δ 8.4 (s, 1), 7.5 (d, 1, J=7.9), 7.36 (d, 1, J=8.3), 7.17 (m, 1), 7.03 (m, 1), 6.9 (s, 1), 6.76 (s, 1), 4.3 (s, 2), 4.23 (m, 4), 2.75 (s, 3).

### EXAMPLE 69

### [4-Chloro-2-(2,3-dihydrobenzofuran-6-yloxy)-5-fluorobenzyl]methylamine p-toluenesulfonic acid salt

Preparation 1 benzaldehyde (221 mg, 1.25 mmol), Preparation 12 phenol (170 mg, 1.25 mmol), K₂CO₃ (518 mg, 3.75 mmol), and DMF (5 mL) were combined and heated at 100° C. for 24 h. The mixture was cooled to rt, poured into H₂O and extracted with EtOAc (2×). The combined extracts were washed with 1M NaOH and brine, dried (MgSO₄), filtered, concentrated, and chromatographed (loaded with CH₂Cl₂; eluted with 10% EtOAc in hexanes). The isolated solid (140 mg) was dissolved in acetonitrile (10 mL) at rt. To the solution was added acetic acid (0.082 mL, 1.44 mmol) and methylamine (0.479 mL of a 2M solution in THF, 0.957 mmol). After 1 h NaBH(OAc)₃ (152 mg, 0.718 mmol) was added. The mixture was stirred at rt for 3 d, poured into sat. NaHCO₃ (50 mL) and 1 M NaOH (5 mL) and extracted with EtOAc (2×50 mL). The combined extracts were dried (MgSO₄), filtered, concentrated, and chromatographed (loaded with CH₂Cl₂; eluted with 10% MeOH in CH₂Cl₂). The resulting yellow gum was dissolved in EtOAc (3 mL) at rt and p-toluenesulfonic acid hydrate (70 mg) was added. After 14 h the mixture was filtered and the resulting solid dried under high vacuum to provide 100 mg of the above named compound as a solid. MS (M⁺)=308, 310. ¹H NMR (400 MHz, CD₃OD): δ 7.69 (d, 2, J=9.1), 7.25 (s, 1), 7.22 (d, 2, J=8.1), 6.90 (d, 1, J=6.4), 6.56 (dd, 1, J=7.9, 2.3), 6.52 (d, 1, J=2.1), 4.62 (t, 2, J=8.7), 4.27 (s, 2), 3.21 (t, 2, J=8.7), 2.76 (s, 3), 2.36 (s, 3).

### EXAMPLE 70

### [4-Chloro-2-(2,3-dihydrobenzofuran-5-yloxy)-5-fluorobenzyl]methylamine p-toluenesulfonic acid salt

Prepared according to the procedure of Example 69 except using Preparation 11 phenol. MS (M⁺)=308, 310. ¹H NMR (400 MHz, CD₃OD): δ 7.68 (d, 2, J=8.1), 7.43 (d, 1, J=8.9), 7.22 (d, 2, J=8.1), 7.02 (bs, 1), 6.87 (dd, 1, J=8.6, 2.5), 6.79 (d, 1, J=1.9), 6.77 (s, 1), 4.60 (t, 2, J=8.8), 4.31 (s, 2), 3.23 (t, 2, J=8.7), 2.78 (s, 3), 2.36 (s, 3).

### EXAMPLE 71

### 1-[3-(2,3-Dihydrobenzofuran-5-yloxy)-4-methylaminomethylphenyl]propan-1-ol

Preparation 2 benzaldehyde (728 mg, 4.00 mmol), Preparation 11 phenol (544 mg, 4.00 mmol), t-butyl-1,1,3,3-tetramethylguanidine (1.60 mL, 8.00 mmol), and DMF (5 mL) were combined and heated at 100° C. for 3 d. The mixture was cooled to rt, diluted with EtOAc, and washed with H₂O, 1M HCl, 1M LiCl, sat. NaHCO₃, and brine. The organic layer was dried (MgSO₄), filtered, concentrated, and chromatographed (loaded with CH₂Cl₂; eluted with 20% EtOAc in hexanes). The isolated solid was dissolved in MeOH (5 mL) at rt. To the solution was added acetic acid (0.157 mL, 2.74 mmol), Na₂SO₄ (313 mg, 2.20 mmol), and methylamine (5.25 mL of a 2M solution in MeOH, 10.5 mmol). After 1H, NaBH₃CN (330 mg, 5.25 mmol) was added. After 4 days at rt, the mixture was concentrated, diluted with 1M NaOH and brine, and extracted with EtOAc (2×). The combined extracts were dried (MgSO₄), filtered, concentrated, and chromatographed (loaded with CH₂Cl₂; eluted with 5-10% MeOH in CH₂Cl₂) to provide 200 mg of the above named compound as a gum. MS (M⁺)=314. ¹H NMR (400 MHz, CDCl₃): δ 7.27 (d, 1, J=7.7), 6.97 (dd, 1, J=7.8, 1.6), 6.86-6.88 (m, 1), 6.70-6.74 (m, 3), 4.58 (t, 2, J=8.7), 4.44 (t, 1, J=6.5), 3.90 (bs, 1), 3.84 (s, 2), 3.18 (t, 2, J=8.6), 2.43 (s, 3), 1.55-1.73 (m, 2), 0.84 (t, 3, J=7.4). Enantiomers were separated using chiral HPLC (Chiralpak AD column; 85/15 heptane/EtOH mobile phase; 0.2% diethylamine modifier).

### EXAMPLE 72

### [2-(2,3-Dihydrobenzofuran-5-yloxy)-5-fluoro-4-methylbenzyl]methylamine formic acid salt

Prepared according to the procedure of Example 62 except using Preparation 3 benzaldehyde and Preparation 5 phenol (1.05 mmol). MS (M⁺)=288.3. ¹H NMR (400 MHz, CDCl₃): δ 7.08 (d, 1, J=9.5), 6.8 (t, 1, J=1), 6.73 (m, 1), 6.7 (s, 1), 6.69 (bs, 1), 4.57 (m, 2), 3.8 (s, 2), 3.18 (m, 2), 2.46 (s, 3), 2.15 (s, 3).

### EXAMPLE 73

### [2-(4-Chloro-2,3-dimethylphenoxy)-5-fluoro-4-methylbenzyl]methylamine formic acid salt

Prepared according to the procedure of Example 62 except using Preparation 3 benzaldehyde and Preparation 20 phenol (1.05 mmol). MS (M⁺)=308.3. ¹H NMR (400 MHz, CD₃OD): δ 8.5 (s, 1), 7.26-7.18 (m, 2), 6.65 (d, 1, J=8.7), 6.46 (d, 1, J=6.5), 6.2 (bs, 1), 4 (s, 2), 2.57 (s, 3), 2.4 (s, 3), 2.23 (s, 3) 2.18 (s, 3).

### EXAMPLE 74

### [4-Chloro-5-fluoro-2-(4-methyl-2,3-dihydrobenzofuran-5-yloxy)benzyl]methylamine

Preparation 1 benzaldehyde (47 mg, 0.27 mmol), Preparation 16 phenol (40 mg, 0.27 mmol), K₂CO₃ (110 mg, 0.80 mmol), and DMF (5 mL) were combined and heated at 85° C. for 14 h. The mixture was cooled to rt, diluted with EtOAc, and washed with H₂O, 1M LiCl, sat. NaHCO₃, and brine. The organic layer was dried (MgSO₄), filtered, concentrated, and chromatographed (loaded with CH₂Cl₂; eluted with 5% EtOAc in hexanes). The isolated solid was dissolved in MeOH (5 mL) at rt. To the solution was added methylamine (0.35 mL of a 2M solution in MeOH, 0.70 mmol) and 4 angstrom molecular sieves (50 mg). After 14 h, NaBH₄ (11 mg, 0.28 mmol) was added. After 2 h at rt, the mixture was filtered and concentrated. To the resulting solid was added 1M NaOH and brine and the mixture was extracted with EtOAc (2×). The combined extracts were dried (MgSO₄), filtered, concentrated, and chromatographed (loaded with CH₂Cl₂; eluted with 5-10% MeOH in CH₂Cl₂) to provide 26.6 mg of the above named compound as a gum. MS (M⁺)=322, 324. ¹H NMR (400 MHz, CDCl₃): δ 7.21 (d, 1, J=9.3), 6.67 (d, 1, J=8.5), 6.60 (d, 1, J=8.5), 6.53 (d, 1, J=6.2), 4.63 (t, 2, J=8.7), 4.55-4.63 (bs, 1), 3.90 (s, 2), 3.17 (t, 2, J=8.7), 2.49 (s, 3), 2.04 (s, 3).

### EXAMPLE 75

### 1-[4-Methylaminomethyl-3-(4-methyl-2,3-dihydrobenzofuran-5-yloxy)phenyl]propan-1-ol

Preparation 2 benzaldehyde (546 mg, 3.00 mmol), Preparation 16 phenol (450 mg, 3.00 mmol), K₂CO₃ (2.41 mL, 12.0 mmol), and DMF (5 mL) were combined and heated at 100° C. for 14 h. The mixture was cooled to rt, diluted with EtOAc, and washed with 1M HCl, 1M LiCl, sat. NaHCO₃, and brine. The organic layer was dried (MgSO₄), filtered, concentrated, and chromatographed (loaded with CH₂Cl₂; eluted with 20% EtOAc in hexanes). The isolated solid was dissolved in MeOH (25 mL) at rt. To the solution was added methylamine (1.60 mL of a 2M solution in MeOH, 3.20 mmol) and 4 angstrom molecular sieves (200 mg). After 2 d, NaBH₄ (49 mg, 1.3 mmol) was added. After 2 h at rt, the mixture was filtered and concentrated. To the resulting solid was added 1M NaOH and brine and the mixture was extracted with EtOAc (2×). The combined extracts were dried (MgSO₄), filtered, concentrated, and chromatographed (loaded with CH₂Cl₂; eluted with 5-10% MeOH in CH₂Cl₂) to provide 159 mg of the above named compound as a solid. MS (M⁺)=328. ¹H NMR (400 MHz, CDCl₃): δ 7.49 (d, 1, J=7.7), 7.06 (dd, 1, J=7.8, 1.6), 6.85 (d, 1, J=8.5), 6.64 (d, 1, J=8.5), 6.63 (s, 1), 4.64 (t, 2, J=8.7), 4.38 (s, 2), 3.23 (t, 2, J=8.7), 2.80 (s, 3), 2.11 (s, 3), 1.55-1.68 (m, 2), 0.82-0.88 (m, 3). Enantiomers were separated using chiral HPLC (Chiralpak AD column; 85/15 heptane/EtOH mobile phase; 0.2% diethylamine modifier).

### EXAMPLE 76

### [2-(Benzofuran-5-yloxy)-4-chloro-5-fluorobenzyl]methylamine formic acid salt

Prepared according to the procedure of Example 62 except using Preparation 1 benzaldehyde and Preparation 15 phenol (1.05 mmol). MS (M⁺)=306.2. ¹H NMR (400 MHz, CDCl₃): δ 7.7 (m, 1), 7.5 (m, 1), 7.3 (s, 1), 7.19 (m, 1), 7.2-6.9 (m, 2), 6.8 (s, 1), 6.1 (bs, 1), 4.14 (s, 2), 2.6 (s, 3).

### EXAMPLE 77

### [4-Chloro-2-(2,3-dihydrobenzofuran-7-yloxy)-5-fluorobenzyl]methylamine formic acid salt

Prepared according to the procedure of Example 62 except using Preparation 1 benzaldehyde and Preparation 14 phenol (1.05 mmol). MS (M⁺)=308.2, 310.2. ¹H NMR (400 MHz, CDCl₃): δ 7.39 (m, 1), 7.12 (m, 1), 6.9-6.87 (m, 2), 6.78 (m, 1), 4.62 (m, 2), 4.1 (s, 2), 3.3 (m, 2), 2.6 (s, 3).

### EXAMPLE 78

### [2-(Benzofuran-7-yloxy)-4-chloro-5-fluorobenzyl]methylamine formic acid salt

Prepared according to the procedure of Example 62 except using Preparation 1 benzaldehyde and Preparation 13 phenol (1.05 mmol). MS (M⁺)=306.2, 308.2. ¹H NMR (400 MHz, CDCl₃); δ 7.64 (s, 1), 7.5-7.4 (m,2), 7.28-7.21 (m, 1), 6.97 (m, 1), 6.88-6.84 (m, 2), 5.4 (bs, 1), 4.07 (s, 2), 2.59 (s, 3).

### EXAMPLE 79

### [2-(Benzofuran-5-yloxy)-5-fluoro-4-methylbenzyl]methylamine formic acid salt

Prepared according to the procedure of Example 62 except using Preparation 3 benzaldehyde and Preparation 15 phenol (1.05 mmol). MS (M⁺)=286.2. ¹H NMR (400 MHz, CDCl₃): δ 8.5 (s, 1), 7.68 (s 1), 7.49 (m, 1), 7-7.3 (m, 3), 6.7 (s, 1), 6.63 (m, 1), 4.1 (s, 2), 2.6 (s, 3), 2.1 (s, 3).

### EXAMPLE 80

### [2-(2,3-Dihydrobenzofuran-6-yloxy)-5-fluoro-4-methylbenzyl]methylamine formic acid salt

Prepared according to the procedure of Example 62 except using Preparation 3 benzaldehyde and Preparation 12 phenol (1.05 mmol). MS (M⁺)=288.3. ¹H NMR (400 MHz, CDCl₃): δ 7.14 (m, 1), 6.8 (m, 1), 6.3-6.5 (m, 3), 4.6 (m, 2), 4.03 (s, 2), 3.2 (m, 2), 2.2 (s, 6).

### EXAMPLE 81

### [2-(2,3-Dihydrobenzofuran-7-yloxy)-5-fluoro-4-methylbenzyl]methylamine formic acid salt

Prepared according to the procedure of Example 62 except using Preparation 3 benzaldehyde and Preparation 14 phenol (1.05 mmol). MS (M⁺)=288.3 ¹H NMR (400 MHz, CDCl₃): δ 7.18 (m, 1), 7.1-7.0 (m, 2), 6.9-6.7 (m, 2), 4.64 (m, 2), 4.2 (s, 2), 3.3 (m, 2), 2.6 (s, 3), 2.2 (s, 3).

### EXAMPLE 82

### [4-Chloro-2-(2,3-dihydrobenzofuran-4-yloxy)-5-fluorobenzyl]methylamine

Prepared according to the procedure of Example 74 except using Preparation 35 phenol. MS (M⁺)=308, 310. ¹H NMR (400 MHz, CDCl₃): δ 7.24 (d, 1, J=9.3), 7.06 (t, 1, J=8.2), 6.88 (d, 1, J=6.4), 6.59 (d, 1, J=7.9), 6.29 (d, 1, J=8.3), 4.60 (t, 2, J=8.7), 3.75 (s, 2), 3.09 (t, 2, J=8.7), 2.43 (s, 3).

### EXAMPLE 83

### [4-Chloro-2-(4-chloro-benzofuran-5-yloxy)-5-fluorobenzyl]methylamine

Preparation 1 benzaldehyde (177 mg, 1.00 mmol), Preparation 17 phenol (168.5 mg, 1.00 mmol), K₂CO₃ (415 mg, 3.00 mmol), and DMF (5 mL) were combined and heated at 85° C. for 14 h. The mixture was cooled to rt, diluted with EtOAc, and washed with H₂O, 1M LiCl, sat. NaHCO₃, and brine. The organic layer was dried (MgSO₄), filtered, concentrated, and chromatographed (loaded with CH₂Cl₂; eluted with 10% EtOAc in hexanes). The isolated solid was dissolved in MeOH (10 mL) at rt. To the solution was added acetic acid (0.112 mL, 1.96 mmol), Na₂SO₄ (225 mg, 1.58 mmol), and methylamine (3.77 mL of a 2M solution in MeOH, 7.54 mmol). After 6 h, NaBH₃CN (237 mg, 3.77 mmol) was added. After 14 h at rt, the mixture was concentrated, diluted with 1M NaOH and brine, and extracted with EtOAc (2×). The combined extracts were dried (MgSO₄), filtered, concentrated, and chromatographed (loaded with CH₂Cl₂; eluted with 5% MeOH in CH₂Cl₂) to provide 98 mg of the above named compound as a gum. MS (M⁺)=340, 342. ¹H NMR (400 MHz, CDCl₃): δ 7.72 (d, 1, J=2.3), 7.44 (dd, 1, J=8.7, 0.8), 7.26 (d, 1, J=7.3), 7.06 (d, 1, J=8.9), 6.90 (dd, 1, J=2.1, 0.8), 6.55 (d, 1, J=6.2), 3.99 (s, 2), 2.56 (s, 3).

### EXAMPLE 84

### [4-Chloro-2-(4-chloro-2,3-dihydrobenzofuran-5-yloxy)-5-fluorobenzyl]methylamine

Prepared according to the procedure of Example 74 except using Preparation 18 phenol and stirring for 4 d after the NaBH₄ addition. MS (M⁺)=342, 344. ¹H NMR (400 MHz, CDCl₃): δ 7.23 (d, 1, J=9.3), 6.85 (d, 1, J=8.5), 6.67 (d, 1, J=8.5), 6.56 (d, 1, J=6.2), 4.67 (t, 2, J=8.8), 4.27 (bs, 1), 3.90 (s, 2), 3.28 (t, 2, J=8.7), 2.48 (s, 3).

### EXAMPLE 85

### [4-Chloro-2-(1H-indol-5-yloxy)benzyl]methylamine

¹H NMR (400 MHz, CDCl₃) δ 8.69 (br s, 1H), 7.36 (d, 1H, J=8.7 Hz), 7.27 (d, 1H, J=8.3 Hz), 7.25-7.23 (m, 2H), 6.98 (dd, 1H, J=7.9, 2.1 Hz), 6.89 (dd, 1H, J=8.7, 2.5 Hz), 6.71 (d, 1H, J=2.1 Hz), 6.51 (d, 1H, J=2.9 Hz), 3.91 (s, 2H), 2.93 (br s, 1H), 2.50 (s, 3H); ¹³C NMR (100 MHz, CDCl₃) δ 158.3, 149.5, 133.9, 133.3, 131.4, 128.8, 127.3, 126.0, 122.3, 116.9, 115.5, 112.4, 111.3, 102.9, 50.5, 35.7; APCI m/z 287.2 (M+1).

### EXAMPLE 86

### [4-Chloro-5-fluoro-2-(1H-indol-5-yloxy)benzyl]methylamine

¹H NMR (400 MHz, CDCl₃) δ 8.88 (br s, 1H), 7.33 (d, 1H, J=8.7 Hz), 7.26-7.19 (m, 3H), 6.87 (dd, 1H, J=8.7, 2.5 Hz), 6.79 (d, 1H, J=6.2 Hz), 6.50 (d, 1H, J=3.3 Hz), 3.87 (s, 2H), 2.50 (s, 3H), 2.19 (br s, 1H); ¹³C NMR (100 MHz, CDCl₃) δ 154.9, 153.3, 152.5, 150.2, 133.2, 130.2, 128.8, 126.2, 119.8, 119.0, 117.6, 117.4, 115.0, 112.5, 110.6, 102.8, 50.3, 35.9; APCI m/z 301.3 (M+1).

### EXAMPLE 87

### [4-Chloro-2-(3-methyl-1H-indol-5-yloxy)benzyl]methylamine

¹H NMR (400 MHz, CDCl₃) δ 8.32 (br s, 1H), 7.31 (d, 1H, J=8.7 Hz), 7.28 (d, 1H, J=7.9 Hz), 7.20 (d, 1H, J=2.5 Hz), 7.01 (s, 1H), 6.97 (dd, 1H, J=7.9, 2.1 Hz), 6.88 (dd, 1H), J=8.7, 2.5 Hz), 3.91 (s, 2H), 2.50 (s, 4H), 2.28 (s, 2H); ¹³C NMR (100 MHz, CDCl₃) δ 158.5, 149.0, 133.7, 131.3, 129.3, 127.5, 123.5, 122.1, 116.6, 115.5, 112.5, 112.3, 112.1, 109.9, 50.6, 35.9, 9.9; APCI m/z 301.3 (M+1).

### EXAMPLE 88

### [4-Chloro-2-(4-fluoro-1-methyl-1H-indol-5-yloxy)benzyl]methylamine

¹H NMR (400 MHz, CDCl₃) δ 7.31 (d, 1H, J=8.3 Hz), 7.10-7.06 (m, 2H), 7.02-6.95 (m, 2H), 6.57-6.54 (m, 2H), 5.58 (br s, 1H), 4.02 (s, 2H), 3.79 (s, 3H), 2.51 (s, 3H); ¹³C NMR (100 MHz, CDCl₃) δ 158.4, 148.8, 146.3, 137.0, 136.9, 135.2, 133.3, 133.2, 132.2, 130.5, 123.1, 122.6, 118.9, 118.7, 117.2, 114.8, 106.1, 106.1, 97.7, 49.2, 34.4, 33.5; APCI m/z 319.1 (M+1).

### EXAMPLE 89

### [4-Chloro-2-(3,4-dihydro-2H-benzo[1,4]oxazin-6-yloxy)benzyl]methylamine

¹H NMR (400 MHz, CDCl₃) δ 7.25 (d, 1H, J=8.3 Hz), 6.97 (dd, 1H, J=8.3, 2.1 Hz), 6.77 (d, 1H, J=2.1 Hz), 6.72 (d, 1H, J=8.3 Hz), 6.26 (d, 1H, J=2.5 Hz), 6.24 (d, 1H, J=2.5 Hz), 4.22 (t, 2H, J=4.4 Hz), 3.78 (t, 2H, J=4.4 Hz), 2.42 (s, 3H); APCI m/z 305.3 (M+1).

### EXAMPLE 90

### [4-Chloro-2-(1-methyl-1H-indol-5-yloxy)benzyl]methylamine

¹H NMR (400 MHz, CDCl₃) δ 7.31 (d, 1H, J=8.7 Hz), 7.29 (d, 1H, J=8.3 Hz), 7.25 (d, 1H, J=2.1 Hz), 7.10 (d, 1H, J=2.9 Hz), 6.98 (dd, 1H, J=7.9, 2.1 Hz), 6.95 (dd, 1H, J=8.7, 2.5 Hz), 6.70 (d, 1H, J=2.1 Hz), 6.45 (d, 1H, J=2.9 Hz), 3.89 (s, 2H), 3.81 (s, 3H), 2.82 (br s, 1H), 2.48 (s, 3H); ¹³C NMR (100 MHz, CDCl₃) δ 158.3, 149.4, 134.2, 133.6, 131.3, 130.4, 129.3, 127.8, 122.3, 116.9, 115.2, 111.5, 110.6, 101.2, 50.5, 35.9, 33.3; APCI m/z 301.2 (M+1).

### EXAMPLE 91

### [4-Chloro-2-(1-methyl-1H-indol-4-yloxy)benzyl]methylamine

¹H NMR (400 MHz, CDCl₃) δ 7.34 (d, 1H, J=8.3 Hz), 7.18-7.16 (m, 2H), 7.04 (dd, 1H, J=8.3, 2.1 Hz), 6.99 (d, 1H, J=2.9 Hz), 6.80 (d, 1H, J=2.1 Hz), 6.67 (dd, 1H, J=6.2, 2.1 Hz), 6.34-6.33 (m, 1H), 3.90 (s, 2H), 3.80 (s, 3H), 2.47 (s, 3H), 2.23 (br s, 1H); ¹³C NMR (100 MHz, CDCl₃) δ 156.8, 149.2, 139.2, 133.6, 131.3, 128.9, 128.7, 123.1, 122.5, 120.9, 118.1, 108.7, 106.1, 98.3, 50.6, 36.0, 33.4; APCI m/z 301.3 (M+1).

### EXAMPLE 92

### [4-Chloro-2-(1-methyl-2,3-dihydro-1H-indol-5-yloxy)benzyl]methylamine

¹H NMR (400 MHz, CDCl₃) δ 7.24 (d, 1H, J=7.9 Hz), 6.95 (dd, 1H, J=7.9, 2.1 Hz), 6.76-6.72 (m, 2H), 6.69 (d, 1H, J=2.1 Hz), 6.43 (d, 1H, J=8.3 Hz), 3.81 (s, 2H), 3.32 (t, 2H, J=8.1 Hz), 2.93 (t, 2H, J=8.1 Hz), 2.76 (s, 3H), 2.44 (s, 3H), 1.95 (br s, 1H); ¹³C NMR (100 MHz, CDCl₃) δ 158.2, 150.7, 147.9, 133.5, 132.5, 131.2, 128.0, 122.1, 119.0, 117.3, 116.5, 107.8, 56.8, 50.7, 36.9, 36.0, 29.0; APCI m/z 303.3 (M+1).

### EXAMPLE 93

### [4-Chloro-2-(2,3-dihydro-1H-indol-5-yloxy)benzyl]methylamine

¹H NMR (400 MHz, CDCl₃) δ 7.23 (d, 1H, J=7.9 Hz), 6.94 (dd, 1H, J=7.9, 2.1 Hz), 6.79-6.78 (m, 1H), 6.69 (d, 1H, J=2.1 Hz), 6.67 (dd, 1H, J=8.3, 2.5 Hz), 6.58 (d, 1H, J=8.3 Hz), 3.80 (s, 2H), 3.57 (t, 2H, J=8.3 Hz), 3.01 (t, 2H, J=8.3 Hz), 2.43 (s, 3H); ¹³C NMR (100 MHz, CDCl₃) δ 158.1, 148.7, 148.4, 133.4, 131.6, 131.1, 128.1, 122.2, 119.0, 117.4, 116.5, 110.1, 50.7, 48.0, 36.1, 30.3; APCI m/z 289.3 (M+1).

### EXAMPLE 94

### [4-Chloro-5-fluoro-2-(1-methyl-1H-indol-5-yloxy)benzyl]methylamine

¹H NMR (400 MHz, CDCl₃) δ 7.30 (d, 1H, J=9.1 Hz), 7.22-7.19 (m, 2H), 7.10 (d, 1H, J=2.9 Hz), 6.93 (dd, 1H, J=8.7, 2.5 Hz), 6.77 (d, 1H, J=6.2 Hz), 6.44 (dd, 1H, J=2.9, 0.8 Hz), 3.85 (s, 2H), 3.80 (s, 3H), 2.47 (s, 3H), 2.12 (br s, 1H); ¹³C NMR (100 MHz, CDCl₃) δ 154.9, 153.3, 152.6, 150.1, 134.1, 130.6, 130.5, 129.3, 119.7, 119.5, 118.9, 117.5, 117.3, 114.6, 110.7, 110.6, 101.1, 50.3, 36.1, 33.3; APCI m/z 319.2 (M+1).

### EXAMPLE 95

### [2-(1H-Indol-5-yloxy)-4-methanesulfonylbenzyl]methylamine

¹H NMR (400 MHz, CDCl₃) δ 8.63 (br s, 1H), 7.58-7.55 (m, 2H), 7.35 (d, 1H, J=8.7 Hz), 7.27-7.23 (m, 3H), 6.86 (dd, 1H, J=8.7, 2.5 Hz), 6.50 (d, 1H, J=1.2 Hz), 4.00 (s, 2H), 2.93 (s, 3H), 2.52 (s, 3H), 2.05 (br s, 1H); ¹³C NMR (100 MHz, CDCl₃) δ 158.3, 148.9, 140.4, 135.9, 133.5, 130.8, 129.0, 126.2, 120.8, 115.3, 114.3, 112.7, 111.5, 103.0, 50.8, 44.6, 36.3; APCI m/z 331.2 (M+1).

### EXAMPLE 96

### 1-[4-Methylaminomethyl-3-(1-methyl-1H-indol-4-yloxy)phenyl]propan-1-ol

¹H NMR (400 MHz, CDCl₃) δ 7.32 (d, 1H, J=7.5 Hz), 7.13-7.07 (m, 2H), 7.04 (dd, 1H, J=7.9, 1.7 Hz), 6.96 (d, 1H, J=2.9 Hz), 6.85 (d, 1H, J=1.3 Hz), 6.52 (dd, 1H, J=6.6, 2.1 Hz), 6.33 (d, 1H, J=3.3 Hz), 4.43 (t, 1H, J=6.4 Hz), 3.81 (s, 2H), 3.79 (s, 3H), 2.38 (s, 3H), 2.33 (br s, 2H), 1.72-1.57 (m, 2H), 0.82 (t, 3H, J=7.5 Hz); ¹³C NMR (100 MHz, CDCl₃) □ 155.6, 150.4, 145.8, 139.1, 130.6, 129.5, 128.5, 122.4, 120.9, 120.5, 116.5, 107.3, 105.1, 98.5, 75.5, 50.8, 35.9, 33.5, 32.0; APCI m/z 325.3 (M+1). Enantiomers were separated using chiral HPLC (Chiralpak AD column (5 cm×50 cm); 82/18 heptane/IPA mobile phase; 0.1% TFA modifier; flow rate=85 mL/min).

### EXAMPLE 97

### [4-Chloro-2-(1,4-dimethyl-2,3-dihydro-1H-indol-5-yloxy)benzyl]methylamine

¹H NMR (400 MHz, CDCl₃) δ 7.23 (d, 1H, J=8.3 Hz), 6.91 (dd, 1H, J=7.9, 2.1 Hz), 6.69 (d, 1H, J=8.3 Hz), 6.51 (d, 1H, J=2.1 Hz), 6.31 (d, 1H, J=8.3 Hz), 3.86 (s, 2H), 3.34 (t, 2H, J=8.1 Hz), 2.89 (t, 2H, J=8.1 Hz), 2.75 (s, 3H), 2.45 (s, 3H), 2.00 (s, 3H), 1.90 (br s, 1H); ¹³C NMR (100 MHz, CDCl₃) δ 158.3, 150.8, 145.4, 133.5, 131.3, 131.1, 127.1, 126.8, 121.5, 120.0, 114.6, 105.4, 56.6, 50.9, 36.9, 36.0, 27.9, 12.9; APCI m/z 317.4 (M+1).

### EXAMPLE 98

### [4-Chloro-5-fluoro-2-(1-methyl-1H-indol-4-yloxy)benzyl]methylamine

¹H NMR (400 MHz, CDCl₃) δ 7.25 (d, 1H, J=7.9 Hz), 7.15-7.12 (m, 2H), 7.01 (d, 1H, J=2.9 Hz), 6.86 (d, 1H, J=6.2 Hz), 6.56 (dd, 1H, J=5.5, 3.1 Hz), 6.33 (d, 1H, J=2.9 Hz), 3.84 (s, 2H), 3.82 (s, 3H), 2.45 (s, 3H), 1.92 (br s, 1H); APCI m/z 319.2 (M+1).

### EXAMPLE 99

### [4-Chloro-5-fluoro-2-(1-methyl-2,3-dihydro-1H-indol-5-yloxy)benzyl]methylamine

¹H NMR (400 MHz, CDCl₃) δ 7.16 (d, 1H, J=9.5 Hz), 6.76-6.73 (m, 2H), 6.70 (dd, 1H, J=8.3, 2.5 Hz), 6.42 (d, 1H, J=8.3 Hz), 3.80 (s, 2H), 3.31 (t, 2H, J=8.1 Hz), 2.92 (t, 2H, J=8.1 Hz), 2.75 (s, 3H), 2.71 (br s, 1H), 2.43 (s, 3H); ¹³C NMR (100 MHz, CDCl₃) δ 154.8, 153.3, 152.4, 150.6, 148.4, 132.5, 130.0, 119.6, 119.4, 118.4, 117.6, 117.4, 116.8, 107.8, 56.8, 50.1, 36.9, 35.9, 29.0; APCI m/z 321.2 (M+1).

### EXAMPLE 100

### 1-[4-Methylaminomethyl-3-(1-methyl-1H-indol-5-yloxy)phenyl]propan-1-ol

¹H NMR (400 MHz, CDCl₃) δ 7.29-7.27 (m, 2H), 7.18 (d, 1H, J=2.1 Hz), 7.07 (d, 1H, J=2.9 Hz); 6.98 (dd, 1H, J=7.9, 1.7 Hz), 6.92 (dd, 1H, J=8.7, 2.5 Hz), 6.73 (d, 1H, J=1.7 Hz), 6.40 (dd, 1H, J=2.9, 0.8 Hz), 4.41 (t, 1H, J=6.6 Hz), 3.82 (s, 2H), 3.80 (s, 3H), 2.42 (s, 3H), 2.25 (br s, 2H), 1.71-1.56 (m, 2H), 0.83 (t, 3H, J=7.5 Hz); ¹³C NMR (100 MHz, CDCl₃) δ 157.2, 150.4, 145.8, 133.9, 130.5, 130.2, 129.2, 128.8, 120.0, 115.0, 114.8, 110.6, 110.4, 101.0, 75.7, 51.0, 35.9, 33.3, 32.1, 10.4; APCI m/z 325.3 (M+1).

### EXAMPLE 101

### [4-Chloro-2-(1,4-dimethyl-1H-indol-5-yloxy)benzyl]methylamine

¹H NMR (400 MHz, CDCl₃) δ 7.27 (d, 1H, J=8.3 Hz), 7.18 (d, 1H, J=8.7 Hz), 7.11 (d, 1H, J=3.3 Hz), 6.92 (dd, 1H, J=8.3, 2.1 Hz), 6.87 (d, 1H, J=8.7 Hz), 6.52 (dd, 1H, J=3.3, 0.8 Hz), 6.43 (d, 1H, J=2.1 Hz), 3.96 (s, 2H), 3.82 (s, 3H), 2.51 (s, 3H), 2.49 (br s, 1H), 2.34 (s, 3H); ¹³C NMR (100 MHz, CDCl₃) δ 158.5, 145.5, 134.2, 133.8, 131.2, 129.8, 129.7, 126.5, 121.8, 121.4, 116.2, 114.6, 108.2, 100.0, 50.7, 35.8, 33.4, 12.6; APCI m/z 315.3 (M+1).

### EXAMPLE 102

### [4-Bromo-2-(1,4-dimethyl-1H-indol-5-yloxy)benzyl]methylamine

¹H NMR (400 MHz, CDCl₃) δ 7.22 (d, 1H, J=8.3 Hz), 7.18 (d, 1H, J=8.7 Hz), 7.20-7.07 (m, 2H), 6.89 (d, 1H, J=8.7 Hz), 6.61 (d, 1H, J=2.1 Hz), 6.53 (dd, 1H, J=2.9, 0.8 Hz), 3.95 (s, 2H), 3.81 (s, 3H), 2.52 (s, 3H), 2.37 (s, 3H), 2.13 (br s, 1H); ¹³C NMR (100 NMR (100 MHz, CDCl₃) δ 158.6, 145.5, 134.2, 131.4, 129.8, 129.7, 127.5, 124.4, 121.7, 121.5, 117.4, 116.2, 108.3, 100.1, 50.9, 36.1, 33.4, 12.6; APCI m/z 361.3, 359.3 (M+1).

### EXAMPLE 103

### 1-[3-(1,4-Dimethyl-1H-indol-5-yloxy)-4-methylaminomethylphenyl]propan-1-ol

¹H NMR (400 MHz, CDCl₃) δ 7.25 (d, 1H, J=7.5 Hz), 7.13 (d, 1H, J=8.7 Hz), 7.09 (d, 1H, J=3.3 Hz), 6.91 (dd, 1H, J=7.5, 1.2 Hz), 6.84 (d, 1H, J=8.7 Hz), 6.50 (dd, 1H, J=3.3, 0.8 Hz), 6.45 (d, 1H, J=1.2 Hz), 4.34 (t, 1H, J=6.6 Hz), 3.89 (s, 2H), 3.80 (s, 3H), 2.44 (s, 3H), 2.35 (s, 3H), 2.27 (br s, 2H), 1.64-1.52 (m, 2H), 0.80 (t, 3H, J=7.3 Hz); ¹³C NMR (100 MHz, CDCl₃) δ 157.8, 146.1, 145.7, 134.0, 130.4, 129.8, 129.5, 127.4, 121.5, 118.8, 116.1, 112.2, 108.0, 99.9, 75.7, 51.3, 35.9, 33.3, 32.1, 12.6, 10.4; APCI m/z 339.3 (M+1). Enantiomers were separated using chiral HPLC (Chiralcel OJ column (5 cm×50 cm); 70/30 heptane/EtOH mobile phase; 0.1% diethylamine modifier; flow rate=85 mL/min).

### EXAMPLE 104

### 7-(5-Chloro-2-methylaminomethylphenoxy)-4H-benzo[1,4]oxazin-3-one

¹H NMR (400 MHz, CDCl₃) δ 7.31 (d, 1H, J=7.9 Hz), 7.05 (dd, 1H, J=8.3, 2.1 Hz), 6.81-6.78 (m, 2H), 6.62 (d, 1H, J=2.5 Hz), 6.57 (dd, 1H, J=8.3, 2.5 Hz), 4.60 (s, 2H), 3.79 (s, 2H), 2.45 (s, 3H); ¹³C NMR (100 MHz, CDCl₃) δ 165.9, 156.1, 153.0, 144.8, 133.9, 131.6, 128.7, 123.9, 122.8, 118.4, 117.0, 113.2, 108.2, 67.3, 50.0, 35.7; APCI m/z 319.3 (M+1).

### EXAMPLE 105

### [4-Chloro-2-(3,4-dihydro-2H-benzo[1,4]oxazin-7-yloxy)benzyl]methylamine

Borane THF (1M, 0.6 mL, 0.6 mmol) was added to a solution of 7-(5-Chloro-2-methylaminomethylphenoxy)-4H-benzo[1,4]oxazin-3-one (Example 115, 0.1 g, 0.3 mmol) in THF (5 mL). The reaction mixture was heated at 60 C for 1 h. MeOH (5 mL) was added and the mixture was heated at 50 C for 18 h. Next 6 N HCl (1 mL) was added and the mixture was heated at 60 C for 2 h. The solvent was removed by evaporation and the residue was partitioned between EtOAc and water. Adjust to pH >10 with sat NaHCO₃. The organic phase was collected, dried and concentrated to afford the title compound (74 mg). ¹H NMR (400 MHz, CDCl₃) δ 7.24 (d, 1H, J=7.9 Hz), 6.97 (dd, 1H, J=7.9, 2.1 Hz), 6.75 (d, 1H, J=2.1 Hz), 6.57 (d, 1H, J=8.7 Hz), 6.48-6.42 (m, 2H), 4.58-4.24 (m, 2H), 3.76 (s, 2H), 3.41 (t, 2H, J=4.6 Hz), 2.43 (s, 3H); ¹³C NMR (100 MHz, CDCl₃) δ 157.4, 148.5, 145.1, 133.5, 131.2, 130.6, 128.3, 122.6, 117.2, 116.5, 112.7, 109.0, 65.7, 50.5, 41.1, 35.9; APCI m/z 305.3 (M+1).

### EXAMPLE 106

### [4-Chloro-2-(7-chloro-1H-indol-4-yloxy)benzyl]methylamine

Ethylene glycol (0.9 mL, 16 mmol) and pTsOH (5 mg) were added to a solution of 4-chloro-2-(4-chloro-3-nitrophenoxy)benzaldehyde (see general procedure above, 0.5 g, 1.6 mmol). This mixture was heated at reflux and the water generated was collected in a Dean-Stark trap. The reaction mixture was diluted with EtOAc and washed with sat NaHCO₃. The organic phase was collected, dried and concentrated to afford 2-[4-chloro-2-(4-chloro-3-nitrophenoxy)phenyl]-[1,3]dioxolane (0.6 g). This material was dissolved in THF (15 mL) and vinyl magnesium bromide (1 M in THF, 5.1 mL, 5.1 mmol) was added using the same method as described in Preparation 31 below to give 7-chloro-4-(5-chloro-2-[1,3]dioxolan-2-yl-phenoxy)-1H-indole (200 mg). This material was dissolved in THF (10 mL) and HOAc (2.5 mL) and water (2.5 mL) were added. The resulting mixture was heated at 60 C for 3 days. The mixture was partitioned between EtOAc and sat aq. NaHCO₃. The organic phase was collected, dried and concentrated to afford 4-chloro-2-(7-chloro-1H-indol-4-yloxy)benzaldehyde (0.18 g). This material was converted to the title compound using the general reductive amination procedure with methyl amine described above. ¹H NMR (400 MHz, CDCl₃) δ 8.88 (br s, 1H), 7.35 (d, 1H, J=8.3 Hz), 7.18 (d, 1H, J=3.3 Hz), 7.10 (d, 1H, J=8.3 Hz), 7.05 (dd, 1H, J=7.9, 2.1 Hz), 6.76 (d, 1H, J=2.1 Hz), 6.59 (d, 1H, J=8.3Hz), 6.42 (d, 1H, J=3.3Hz), 3.90 (s, 2H), 2.47 (br s, 4H); ¹³C NMR (100 MHz, CDCl₃) δ 156.5, 148.0, 135.1, 133.9, 131.5, 128.0, 125.0, 123.5, 121.9, 121.5, 118.1, 112.8, 109.8, 101.1, 50.2, 35.7; APCI m/z 323.3, 321.3 (M+1).

### EXAMPLE 107

### [4-Chloro-2-(1-methyl-1,2,3,4-tetrahydroquinolin-6-yloxy)benzyl]methylamine

¹H NMR (400 MHz, CDCl₃) δ 7.23 (d, 1H, J=8.3 Hz), 6.95 (dd, 1H, J=8.3, 2.1 Hz), 6.74 (dd, 1H, J=8.7, 2.9 Hz), 6.71 (d, 1H, J=2.9 Hz), 6.65 (d, 1H, J=2.9 Hz), 6.56 (d, 1H, J=8.7 Hz), 3.82 (s, 2H), 3.21 (t, 2H, J=5.8 Hz), 2.88 (s, 3H), 2.75 (t, 2H, J=6.6 Hz), 2.44 (s, 3H), 2.02-1.96 (m, 3H); ¹³C NMR (100 MHz, CDCl₃) δ 158.1, 146.2, 144.2, 133.5, 131.1, 128.0, 124.8, 122.1, 120.8, 118.7, 116.5, 112.1, 51.5, 50.7, 39.7, 36.0, 28.1, 22.6; APCI m/z 317.3 (M+1).

### EXAMPLE 108

### [4-Chloro-2-(1,4-dimethyl-2,3-dihydro-1H-indol-5-yloxy)-5-fluorobenzyl]methylamine

¹H NMR (400 MHz, CDCl₃) δ 7.16 (d, 1H, J=9.1 Hz), 6.65 (d, 1H, J=8.3 Hz), 6.55 (d, 1H, J=6.2 Hz), 6.29 (d, 1H, J=8.3 Hz), 3.88 (s, 2H), 3.50 (br s, 1H), 3.34 (t, 2H, J=8.3 Hz), 2.89 (t, 2H, J=8.3 Hz), 2.75 (s, 3H), 2.48 (s, 3H), 2.00 (s, 3H); ¹³C NMR (100 MHz, CDCl₃) δ 154.3, 153.6, 151.9, 150.8, 145.7, 131.4, 127.9, 127.8, 126.6, 119.7, 119.6, 117.8, 117.6, 116.2, 105.4, 56.5, 50.0, 36.8, 35.6, 27.9, 12.8; APCI m/z 335.3 (M+1).

### EXAMPLE 109

### [4-Chloro-2-(7-chloro-1-methyl-1H-indol-4-yloxy)benzyl]methylamine

¹H NMR (400 MHz, CDCl₃) δ 7.32 (d, 1H, J=7.9 Hz), 7.06-7.02 (m, 2H), 6.92 (d, 1H, J=3.3 Hz), 6.75 (d, 1H, J=1.7 Hz), 6.49 (d, 1H, J=8.3 Hz), 6.33 (d, 1H, J=2.9 Hz), 4.14 (s, 3H), 3.85 (s, 2H), 2.45 (s, 3H), 2.24 (br s, 1H); ¹³C NMR (100 MHz, CDCl₃) δ 156.4, 148.1, 133.8, 133.6, 131.6, 131.3, 128.7, 123.8, 123.5, 123.4, 118.0, 112.9, 109.2, 98.7, 50.4, 36.8, 36.0; APCI m/z 335.1 (M+1).

### EXAMPLE 110

### [4-Chloro-2-(1,4-dimethyl-1H-indol-5-yloxy)-5-fluorobenzyl]methylamine

¹H NMR (400 MHz, CDCl₃) δ 7.20-7.16 (m, 2H), 7.12 (d, 1H, J=3.3 Hz), 6.85 (d, 1H, J=8.7 Hz), 6.53-6.51 (m, 1H), 6.47 (d, 1H, J=6.6 Hz), 3.93 (s, 2H), 3.82 (s, 3H), 2.51 (s, 3H), 2.36 (s, 3H), 1.82 (br s, 1H); ¹³C NMR (100 MHz, CDCl₃) δ 154.3, 153.8, 151.9, 145.9, 134.2, 129.8, 129.8, 129.1, 121.6, 119.3, 119.2, 117.5, 117.2, 116.0, 115.9, 108.3, 100.0, 50.5, 36.1, 33.4, 12.5; APCI m/z 333.3 (M+1).

### EXAMPLE 111

### [4-Chloro-2-(1H-indol-4-yloxy)benzyl]methylamine

¹H NMR (400 MHz, CD₃OD) δ 7.49 (d, 1H, J=7.9 Hz), 7.35 (d, 1H, J=8.3 Hz), 7.24 (d, 1H, J=3.3 Hz), 7.16 (d, 1H, J=7.9 Hz), 7.13 (dd, 1H, J=8.3, 2.1 Hz), 6.77 (d, 1H, J=7.9 Hz), 6.62 (d, 1H, J=1.7 Hz), 6.22 (dd, 1H, J=3.3, 0.8 Hz), 4.36 (s, 2H), 2.77 (s, 3H); APCI m/z 287.1 (M+1).

### EXAMPLE 112

### [4-Chloro-2-(1,4-dimethyl-1H-indol-5-yloxy)-5-methylbenzyl]methylamine

¹H NMR (400 MHz, CDCl₃) δ 7.21 (s, 1H), 7.17 (d, 1H, J=8.7 Hz), 7.11 (d, 1H, J=2.9 Hz), 6.87 (d, 1H, J=8.7 Hz), 6.52 (dd, 1H, J=3.3, 0.8 Hz), 6.47 (s, 1H), 3.92 (s, 2H), 3.80 (s, 3H), 2.52 (s, 3H), 2.37 (s, 3H), 2.30 (s, 3H), 2.03 (br s, 1H); ¹³C NMR (100 MHz, CDCl₃) δ156.4, 146.0, 134.1, 133.2, 132.3, 129.8, 129.6, 128.6, 127.1, 121.6, 116.1, 115.1, 108.1, 100.0, 50.9, 36.1, 33.3, 19.3, 12.6; APCI m/z 329.3 (M+1).

### EXAMPLE 113

### [4-Chloro-2-(1-ethyl-2,3-dihydro-1H-indol-5-yloxy)benzyl]methylamine

¹H NMR (400 MHz, CDCl₃) δ 7.23 (d, 1H, J=7.9 Hz), 6.94 (dd, 1H, J=7.9, 2.1 Hz), 6.76, (d, 1H, J=2.1 Hz), 6.72 (dd, 1H, J=8.3, 2.5 Hz), 6.69 (d, 1H, J=2.1 Hz), 6.43 (d, 1H, J=8.3 Hz), 3.80 (s, 2H), 3.35 (t, 2H, J=8.3 Hz), 3.15 (q, 2H, J=8.3 Hz), 2.94 (t, 2H, J=8.3 Hz), 2.44 (s, 3H), 1.72 (br s, 1H), 1.20 (t, 3H, J=7.1 Hz); ¹³C NMR (100 MHz, CDCl₃) δ 158.2, 149.7, 147.6, 133.4, 132.4, 131.1, 128.1, 122.1, 119.0, 117.4, 116.4, 107.7, 52.9, 50.8, 43.8, 36.1, 28.8.

### EXAMPLE 114

### [4-Chloro-2-(4-methyl-3,4-dihydro-2H-benzo[1.4]oxazin-7-yloxy)benzyl]methylamine

¹H NMR (400 MHz, CDCl₃) δ 7.25 (d, 1H, J=7.1 Hz), 6.97 (dd, 1H, J=7.9, 2.1 Hz), 6.75 (d, 1H, J=2.1 Hz), 6.62 (d, 1H, J=8.3 Hz), 6.50 (dd, 1H, J=8.7, 2.5 Hz), 6.47 (d, 1H, J=2.5 Hz), 4.33-4.30 (m, 2H), 3.80 (s, 2H), 3.24-3.22 (m, 2H), 2.87 (s, 3H), 2.43 (s, 3H), 2.04 (br s, 1H); ¹³C NMR (100 MHz, CDCl₃) δ 157.5, 148.1, 145.4, 133.8, 133.5, 131.2, 122.6, 117.2, 113.4, 112.5, 108.3, 65.5,50.5, 49.3, 39.4, 35.9; APCI m/z 319.3 (M+1).

### EXAMPLE 115

### [4-Chloro-2-(1,4-dimethyl-2,3-dihydro-1H-indol-5-yloxy)-5-methylbenzyl]methylamine

¹H NMR (400 MHz, CDCl₃) δ 7.20 (s, 1H), 6.67 (d, 1H, J=8.3 Hz), 6.53 (s, 1H), 6.29 (d, 1H, J=8.3 Hz), 4.50 (br s, 1H), 3.95 (s, 2H), 3.34 (t, 2H, J=8.1 Hz), 2.89 (t, 2H, J=8.1 Hz), 2.75 (s, 3H), 2.52 (s, 3H), 2.28 (s, 3H), 1.99 (s, 3H); ¹³C NMR (100 MHz, CDCl₃) δ 156.3, 150.8, 145.5, 134.4, 132.9, 131.3, 129.1, 126.7, 123.8, 119.8, 115.2, 105.4, 56.6, 49.7, 36.9, 34.8, 27.9, 19.3, 12.9; APCI m/z 331.4 (M+1).

### EXAMPLE 116

### [4-Chloro-2-(1,3-dimethyl-1H-indol-5-yloxy)benzyl]methylamine

¹H NMR (400 MHz, CDCl₃) δ 7.30-7.25 (m, 2H), 7.21 (d, 1H, J=2.5 Hz), 6.97 (dd, 1H, J=8.1, 2.1 Hz), 6.93 (dd, 1 h, J=8.7, 2.1 Hz), 6.88 (s, 1H), 6.69 (d, 1H, J=2.1 Hz), 3.90 (s, 2H), 3.75 (s, 3H), 2.50 (s, 3H), 2.37 (br s, 1H), 2.29 (s, 3H); ¹³C NMR (100 MHz, CDCl₃) δ 158.6, 148.7, 134.6, 133.6, 131.2, 129.5, 128.2, 127.9, 122.1, 116.5, 115.1, 110.4, 110.1, 50.7, 36.0, 33.0, 9.8; APCI m/z 315.2 (M+1).

### EXAMPLE 117

### [4-Chloro-2-(1,3-dimethyl-2,3-dihydro-1H-indol-5-yloxy)benzyl]methylamine

¹H NMR (400 MHz, CDCl₃) δ 7.23 (d, 1H, J=8.3 Hz), 6.95-6.93 (m, 1H), 6.74-6.71 (m, 2H), 6.69 (d, 1H, J=2.1 Hz), 6.42 (d, 1H, J=8.7 Hz), 3.81 (s, 2H), 3.53 (t, 1H, J=8.7 Hz), 3.36-3.23 (m, 1H), 2.81 (t, 1H, J=8.7 Hz), 2.73 (s, 3H), 2.44 (s, 3H), 1.91 (br s, 1H), 1.28 (d, 3H, J=7.1 Hz); ¹³C NMR (100 MHz, CDCl₃) δ 158.2, 150.3, 148.0, 137.5, 131.1, 128.1, 122.1, 119.1, 116.4, 116.0, 107.9, 64.8, 50.7, 36.8, 36.1, 35.6, 18.3; APCI m/z 317.3 (M+1).

### EXAMPLE 118

### 6-(5-Chloro-2-methylaminomethylphenoxy)-3H-benzooxazol-2-one

Trifluoroacetic anhydride (0.21 mL, 1.5 mmol) and triethylamine (0.16 mL, 1.5 mmol) were added to a solution of [2-(3-benzyloxy-4-nitrophenoxy)-4-chlorobenzyl]methylamine (0.5 g, 1.25 mmol, prepared by the general procedure above starting with 3-benzyloxy-4-nitrophenol—see Preparation 30 below) in methylene chloride (10 mL). The mixture stirred for 2 h at rt at which time water was added. The organic phase was collected, dried and concentrated to afford N-[2-(3-benzyloxy-4-nitrophenoxy)-4-chlorobenzyl]-2,2,2-trifluoro-N-methyl-acetamide (500 mg). This material was subjected to hydrogenolysis conditions (see Preparation 20 below) to afford N-[2-(4-amino-3-hydroxy-phenoxy)-4-chlorobenzyl]-2,2,2-trifluoro-N-methyl-acetamide (0.3 g). This material (250 mg, 0.67 mmol) was dissolved in THF (20 mL) and reacted with carbonyl diiimidazole (120 mg, 0.75 mmol) at reflux for 90 min. The resulting mixture was partitioned between EtOAc and water. The organic phase was collected and concentrated to afford N-[4-chloro-2-(2-oxo-2,3-dihydrobenzooxazol-6-yloxy)benzyl]-2,2,2-trifluoro-N-methyl-acetamide (0.2 g). This material was dissolved in a mixture of MeOH/water (20 mL/1 mL) and potassium carbonate was added (280 mg, 2 mmol). The reaction mixture was heated at reflux for 30 min and then the solvent was removed by evaporation. The residue was partitioned between EtOAc and water and the organic phase was collected and concentrated. The crude material was purified by chromatography (elution with 10:1 chloroform/MeOH) to afford the title compound (0.12 g). ¹H NMR (400 MHz, CDCl₃) δ 7.30 (d, 1H, J=8.3 Hz), 7.02 (dd, 1H, J=7.9, 2.1 Hz), 6.86 (d, 1H, J=8.7 Hz), 6.85 (d, 1H, J=2.5 Hz), 6.73 (dd, 1H, J=8.7, 2.5 Hz), 6.71 (d, 1H, J=2.1 Hz), 6.51 (br s, 2H), 3.88 (s, 2H), 2.52 (s, 3H); ¹³C NMR (100 MHz, CDCl₃) δ157.4, 156.9, 151.2, 145.0, 134.4, 131.9, 128.1, 127.3, 123.6, 117.6, 115.0, 110.7, 102.8, 50.0, 35.3; APCI m/z 305.3 (M+1).

### EXAMPLE 119

### [4-Chloro-2-(1-isopropyl-2,3-dihydro-1H-indol-5-yloxy)benzyl]methylamine

¹H NMR (400 MHz, CDCl₃) δ 7.21 (d, 1H, J=8.3 Hz), 6.93 (dd, 1H, J=8.3, 2.1 Hz), 6.75-6.70 (m, 2H), 6.69 (d, 1H, J=2.1 Hz), 6.37 (d, 1H, J=8.3 Hz), 3.82-3.76 (m, 3H), 3.35 (t, 2H, J=8.3 Hz), 2.92 (t, 2H, J=8.3 Hz), 2.43 (s, 3H), 1.91 (br s, 1H), 1.16 (d, 6H, J=6.6 Hz); ¹³C NMR (100 MHz, CDCl₃) δ 158.4, 148.6, 146.9, 133.4, 132.4, 131.1, 127.9, 121.9, 119.1, 117.5, 116.3, 107.6, 50.8, 46.4, 46.0, 36.0, 28.4, 18.2; APCI m/z 331.3 (M+1).

### EXAMPLE 120

### [2-(Benzothiazol-6-yloxy)-4-chlorobenzyl]methylamine

¹H NMR (400 MHz, CDCl₃) δ 8.91 (s, 1H), 8.08 (d, 1H, J=8.7 Hz), 7.46 (d, 1H, J=2.5 Hz), 7.36 (d, 1H, J=8.3 Hz), 7.19 (dd, 1H, J=8.7, 2.5 Hz), 7.10 (dd, 1H, J=8.3, 2.1 Hz), 6.85 (d, 1H, J=2.1 Hz), 3.78 (s, 2H), 2.42 (s, 3H), 2.24 (br s, 1H); ¹³C NMR (100 MHz, CDCl₃) δ 155.8, 155.1, 153.5, 150.0, 135.4, 133.8, 131.6, 129.7, 124.9, 124.3, 119.2, 118.6, 110.9, 50.2, 36.1; APCI m/z 305.1 (M+1).

### EXAMPLE 121

### [4-Chloro-2-(1-ethyl-4-methyl-1H-indol-5-yloxy)-5-fluorobenzyl]methylamine

¹H NMR (400 MHz, CDCl₃) δ 7.22-7.18 (m, 3H), 6.84 (d, 1H, J=9.1 Hz), 6.54 (d, 1H, J=2.9 Hz), 6.51 (d, 1H, J=6.6 Hz), 4.19 (q, 2H, J=7.5 Hz), 3.93 (s, 2H), 2.52 (s, 3H), 2.37 (s, 3H), 1.93 (br s, 1H), 1.50 (t, 3H, J=7.5 Hz); ¹³C NMR (100 MHz, CDCl₃) δ 154.3, 153.8, 151.9, 145.9, 133.1, 130.0, 129.1, 129.0, 128.0, 121.7, 119.4, 119.2, 117.5, 117.2, 116.0, 115.8, 1048.3, 100.1, 50.6, 41.5, 36.1, 15.8, 12.6; APCI m/z 347.3 (M+1).

### EXAMPLE 122

### [4-Chloro-2-(1,3-dimethyl-2,3-dihydro-1H-indol-5-yloxy)-5-fluorobenzyl]methylamine

¹H NMR (400 MHz, CDCl₃) δ 7.16 (d, 1H, J=9.5 Hz), 6.74 (d, 1H, J=6.6 Hz), 6.71-6.88 (m, 2H), 6.42 (d, 1H, J=8.3 Hz), 3.79 (s, 2H), 3.54 (t, 1H, J=8.3 Hz), 3.29-3.25 (m, 1H), 2.81 (t, 1H, J=8.7 Hz), 2.73 (s, 3H), 2.44 (s, 3H), 2.27 (br s, 1H), 1.28 (d, 3H, J=6.6 Hz); ¹³C NMR (100 MHz, CDCl₃) δ 154.8, 153.3, 152.4, 150.2, 148.6, 137.6, 130.0, 128.2, 119.7, 119.5, 118.5, 118.3, 117.5, 117.3, 115.5, 107.9, 64.7, 50.3, 36.8, 36.0, 35.6, 18.3; APCI m/z 335.3 (M+1).

### EXAMPLE 123

### [4-Chloro-2-(1-trifluoromethyl-2,3-dihydro-1H-indol-5-yloxy)benzyl]methylamine

¹H NMR (400 MHz, CDCl₃) δ 7.29 (d, 1H, J=8.3 Hz), 7.02 (dd, 1H, J=8.3, 2.1 Hz), 6.98 (dd, 1H, J=8.7, 1,2 Hz), 6.84 (t, 1H, J=1.2 Hz), 6.79 (dd, 1H, J=8.7, 2.5 Hz), 6.74 (d, 1H, J=2.1 Hz), 3.78 (s, 2H), 3.75 (t, 2H, J=8.3 Hz), 3.09 (t, 2H, J=8.3 Hz), 2.44 (s, 3H), 1.68 (br s, 1H); ¹³C NMR (100 MHz, CDCl₃) δ 156.9, 152.0, 139.9, 133.5, 133.3, 131.3, 129.1, 124.4, 123.2, 121.9, 118.7, 117.7, 116.8, 113.5, 113.4, 50.5, 48.5, 36.2, 28.5; APCI m/z 357.3 (M+1).

### EXAMPLE 124

### [4-Methanesulfonyl-2-(1-methyl-1H-indol-5-yloxy)benzyl]methylamine

¹H NMR (400 MHz, CDCl₃) δ 7.59-7.54 (m, 2H), 7.32 (d, 1H, J=8.7 Hz), 7.25-7.20 (m, 2H), 7.11 (d, 1H, J=2.9 Hz), 6.93 (dd, 1H, J=8.7, 2.1 Hz), 6.44 (d, 1H, J=2.9 Hz), 3.99 (s, 2H), 3.82 (s, 3H), 2.92 (s, 3H), 2.51 (s, 3H), 1.90 (br s, 1H); ¹³C NMR (100 MHz, CDCl₃) δ 158.4, 148.7, 140.3, 136.0, 134.5, 130.7, 130.6, 129.4, 120.8, 115.0, 114.3, 111.7, 110.9, 101.3, 50.8, 44.6, 36.3, 33.4; APCI m/z 345.3 (M+1).

### EXAMPLE 125

### 7-(5-Chloro-2-methylaminomethylphenoxy)-4-methyl-4H-benzo[1,4]oxazin-3-one

¹H NMR (400 MHz, CDCl₃) δ 7.26 (d, 1H, J=7.9 Hz), 7.01 (dd, 1H, J=8.3, 2.1 Hz), 6.86 (d, 1H, J=8.3 Hz), 6.76 (d, 1H, J=1.7 Hz), 6.61-6.57 (m, 2H), 4.55 (s, 2H), 3.69 (s, 2H), 3.29 (s, 3H), 2.36 (s, 3H), 1.69 (br s); ¹³C NMR (100 MHz, CDCl₃) δ 164.0, 155.7, 153.0, 146.3, 133.5, 131.4, 129.7, 125.9, 118.8, 115.7, 112.7, 108.0, 67.7, 50.2, 36.1, 28.3; APCI m/z 333.3 (M+1).

### EXAMPLE 126

### 6-(5-Chloro-2-methylaminomethylphenoxy)-1H-quinolin-2-one

¹H NMR (400 MHz, CDCl₃) δ 7.71 (d, 1H, J=9.5 Hz), 7.50 (d, 1H, J=8.7 Hz), 7.35 (d, 1H, J=8.3 Hz), 7.23 (dd, 1H, J=9.1, 2.5 Hz), 7.13 (s, 1H, J=2.5 Hz), 7.05 (dd, 1H, J=8.3, 2.1 Hz), 6.74 (d, 1H, J=2.1 Hz), 6.72 (d, 1H, J=9.5 Hz), 3.85 (s, 2H), 2.47 (s, 3H); ¹³C NMR (100 MHz, CDCl₃) δ 164.6, 156.4, 151.5, 140.5, 135.5, 134.0, 131.7, 128.2, 123.8, 123.3, 122.5, 120.9, 118.4, 118.0,

### EXAMPLE 127

### [2-(4-chloro-2,3-dimethylphenoxy)-4-cyclopropylbenzyl]methylamine

¹H NMR (400 MHz, CDCl₃) δ 7.24 (d, 1H, J=7.9 Hz), 7.13 (d, 1H, J=8.7 Hz), 6.74 (dd, 1H, J=7.9, 1.7 Hz), 6.57 (d, 1H, J=9.1 Hz), 6.40 (d, 1H, J=1.7 Hz), 3.76 (s, 2H), 2.42 (s, 3H), 2.38 (s, 3H), 2.24 (s, 3H), 1.80-1.73 (m, 2H), 0.92-0.87 (m, 2H), 0.60-0.56 (m, 2H); ¹³C NMR (100 MHz, CDCl₃) δ 155.4, 153.6, 145.1, 130.6, 129.7, 129.2, 127.4, 127.2, 120.2, 116.8, 115.2, 50.7, 36.0, 17.1, 15.4, 13.4, 9.6; ES m/z 316.0 (M+).

### EXAMPLE 128

### [4-sec-Butyl-2-(4-chloro-2,3-dimethylphenoxy)benzyl]methylamine

¹H NMR (400 MHz, CDCl₃) δ 7.29 (d, 1H, J=7.5 Hz), 7.12 (d, 1H, J=8.7 Hz), 6.90 (dd, 1H, J=7.5, 1.3 Hz), 6.55 (d, 1H, J=8.7 Hz), 6.50 (d, 1H, J=1.3 Hz), 3.76 (s, 2H), 2.51-2.45 (m, 1H), 2.43 (s, 3H), 2.38 (s, 3H), 2.25 (s, 3H), 1.78 (br s, 1H), 1.49 (quin, 2H, J=7.5 Hz), 1.14 (d, 3H, J=6.6 Hz), 0.77 (t, 3H, J=7.5 Hz); ¹³C NMR (100 MHz, CDCl₃) δ 155.1, 153.9, 148.8, 136.4, 130.5, 129.5, 129.0, 127.9, 127.1, 122.1, 116.6, 116.4, 50.8, 41.6, 36.2, 31.3, 21.9, 17.1, 13.4, 12.4.

### EXAMPLE 129

### [2-(4-Chloro-2,3-dimethylphenoxy)-4-isopropyl-benzyl]methylamine

¹H NMR (400 MHz, CDCl₃) δ 7.29 (d, 1H, J=7.9 Hz), 7.13 (d, 1H, J=8.7 Hz), 6.95 (dd, 1H, J=7.9, 1.7 Hz), 6.57 (d, 1H, J=9.1 Hz), 6.55 (d, 1H, J=2.1 Hz), 3.77 (s, 2H), 2.79 (sept, 1H, J=7.1 Hz), 2.43 (s, 3H), 2.39 (s, 3H), 2.25 (s, 3H), 1.79 (br s, 1H), 1.16 (d, 6H, J=7.1 Hz); ¹³C NMR (100 MHz, CDCl₃) δ 155.2, 153.8, 150.0, 136.4, 130.5, 129.5, 129.0, 127.9, 127.2, 121.3, 116.5, 116.0,

### EXAMPLE 130

### 3-[3-(4-Chlorophenoxy)-4-methylaminomethylphenyl]propionitrile

¹H NMR (400 MHz, CDCl₃) δ 7.37 (d, 1H, J=7.9 Hz), 7.30-7.28 (m, 2H), 6.99 (dd, 1H, J=7.9, 1.7 Hz), 6.93-6.90 (m, 2H), 6.71 (d, 1H, J=1.7 Hz), 3.81 (s, 2H), 3.53 (br s, 1H), 2.86 (t, 2H, J=7.3 Hz), 2.57 (t, 2H, J=7.3 Hz), 2.44 (s, 3H); APCI m/z 301.2 (M+1).

### EXAMPLE 131

### [4-(1-Methoxy-butyl)-2-(1-methyl-1H-indol-4-yloxy)benzyl]methylamine

¹H NMR (400 MHz, CDCl₃) δ 7.41 (d, 1H, J=7.5 Hz), 7.14-7.12 (m, 2H), 7.01 (dd, 1H, J=7.5, 1.7 Hz), 6.96 (d, 1H, J=3.3 Hz), 6.79 (d, 1H, J=1.7 Hz), 6.61 (d, 1H, J=5.4, 2.9 Hz), 6.25 (d, 1H, J=3.3 Hz), 5.02 (br s, 1H), 4.01 (s, 2H), 3.95 (dd, 1H, J=7.3, 6.0 Hz), 3.80 (s, 3H), 3.10 (s, 3H), 2.52 (s, 3H), 1.70-1.59 (m, 1H), 1.50-1.40 (m, 1H), 1.31-1.12 (m, 2H), 0.82 (t, 3H, J=7.3 Hz); APCI m/z 353.3 (M+1).

### EXAMPLE 132

### [3-(4-Chloro-2,3-dimethylphenoxy)-4-methylaminomethylphenyl]-acetonitrile

¹H NMR (400 MHz, CDCl₃) δ 7.38 (d, 1H, J=7.9 Hz), 7.17 (d, 1H, J=8.7 Hz), 7.02 (dd, 1H, J=7.9, 1.7 Hz), 6.62 (d, 1H, J=8.7 Hz), 6.51 (d, 1H, J=1.7 Hz), 3.83 (s, 2H), 3.61 (s, 2H), 2.45 (s, 3H), 2.37 (s, 3H), 2.19 (s, 3H), 1.81 (br s, 1H); ¹³C NMR (100 MHz, CDCl₃) δ 156.3, 152.6, 136.9, 131.2, 130.4, 130.3, 130.2, 130.0, 127.5, 122.4, 117.8, 116.0, 50.6, 36.1, 23.5, 17.2, 13.5; APCI m/z 315.2 (M+1).

### EXAMPLE 133

### [2-(1,4-Dimethyl-1H-indol-5-yloxy)-4-(1-methoxypropyl)benzyl]methylamine

¹H NMR (400 MHz, CDCl₃) δ 7.31 (d, 1H, J=7.5 Hz), 7.14 (d, 1H, J=8.7 Hz), 7.09 (d, 1H, J=2.9 Hz), 6.89-6.85 (m, 2H), 6.50 (d, 1H, J=2.5 Hz), 6.41 (d, 1H, J=1.2 Hz), 3.95 (s, 2H), 3.82-3.79 (m, 4H), 3.10 (s, 3H), 2.52 (s, 3H), 2.36 (s, 3H), 2.13 (br s, 1H), 1.68-1.61 (m, 1H), 1.52-1.45 (m, 1H), 0.77 (t, 3H, J=7.5 Hz); ¹³C NMR (100 MHz, CDCl₃) δ 157.8, 146.2, 142.9, 134.0, 130.3, 129.8, 129.4, 127.7, 121.4, 119.8, 116.0, 113.0, 108.0, 99.9, 85.3, 56.7, 51.4, 36.2, 33.3, 31.1, 12.6,10.4; ES m/z 353.1 (M+1).

### EXAMPLE 134

### [2-(1,4-Dimethyl-1H-indol-5-yloxy)-4-(1-methoxy-butyl)benzyl]methylamine

¹H NMR (400 MHz, CDCl₃) δ 7.31 (d, 1H, J=7.9 Hz), 7.14 (d, 1H, J=8.7 Hz), 7.09 (d, 1H, J=3.3 Hz), 6.88-6.84 (m, 2H), 6.50 (d, 1H, J=3.3 Hz), 6.41 (d, 1H, J=1.2 Hz), 3.96 (s, 2H), 3.88 (dd, 1H, J=7.5, 5.8 Hz), 3.81 (s, 3H), 3.08 (s, 3H), 2.52 (s, 3H), 2.45 (br s, 1H), 2.35 (s, 3H), 1.65-1.55 (m, 1H), 1.50-1.38 (m, 1H), 1.32-1.25 (m, 1H), 1.22-1.10 (m, 1H), 0.81 (t, 3H, J=7.5 Hz); ¹³C NMR (100 MHz, CDCl₃) δ 157.8, 146.2, 143.3, 134.0, 130.3, 129.8, 129.4, 127.3, 121.4, 119.7, 116.0, 112.9, 108.0, 99.9, 83.7, 56.7, 51.3, 40.4, 36.0, 33.3, 19.2, 14.1, 12.6; APCI m/z 367.2 (M+1).

### EXAMPLE 135

### 1-[3-(1,4-Dimethyl-1H-indol-5-yloxy)-4-methylaminomethylphenyl]-butan-1-ol

¹H NMR (400 MHz, CDCl₃) δ 7.35 (d, 1H, J=7.9 Hz), 7.15 (d, 1H, J=8.7 Hz), 7.09 (d, 1H, J=2.9 Hz), 6.96 (d, 1H, J=8.7 Hz), 6.91 (dd, 1H, J=7.9, 1.2 Hz), 6.49-6.47 (m, 1H), 6.43 (d, 1H, J=1.7 Hz), 4.36 (dd, 1H, J=7.5, 5.4 Hz), 4.25 (s, 2H), 3.77 (s, 3H), 2.65 (s, 3H), 2.28 (s, 3H), 1.55-1.35 (m, 2H), 1.30-1.05 (m, 2H), 0.78 (t, 3H, J=7.5 Hz); ¹³C NMR (100 MHz, CDCl₃) δ 158.3, 149.3, 144.9, 134.4, 132.3, 129.8, 129.7, 121.7, 119.5, 116.8, 116.5, 112.0, 108.5, 100.1, 74.0, 48.6, 41.2, 33.4, 33.0, 19.1, 14.0, 12.8; APCI m/z 353.2 (M+1).

### EXAMPLE 136

### [2-(4-Chloro-2,3-dimethylphenoxy)-4-isobutyl-benzyl]methylamine

¹H NMR (400 MHz, CDCl₃) δ 7.27 (d, 1H, J=7.5 Hz), 7.13 (d, 1H, J=8.7 Hz), 6.85 (dd, 1H, J=7.5, 1.7 Hz), 6.57 (d, 1H, J=8.7 Hz), 6.44 (d, 1H, J=1.7 Hz), 3.78 (s, 2H), 2.44 (s, 3H), 2.38 (s, 3H), 2.35 (d, 2H, J=7.5 Hz), 2.24 (s, 3H), 1.78-1.72 (m, 2H), 0.84 (d, 6H, J=6.6 Hz); ¹³C NMR (100 MHz, CDCl₃) δ 155.2, 153.7, 142.6, 136.4, 130.3, 129.7, 129.1, 127.6, 127.2, 124.1, 118.2, 116.8, 50.8, 45.2, 36.1, 30.4, 22.5, 17.1, 13.4; ES m/z 332.0 (M+).

### EXAMPLE 137

### [4-Chloro-2-(1-methyl-1H-indol-7-yloxy)benzyl]methylamine

¹H NMR (400 MHz, CDCl₃) δ 7.43 (dd, 1H, J=7.9, 0.8 Hz), 7.38 (d, 1H, J=7.9 Hz), 7.04 (dd, 1H, J=8.3, 2.1 Hz), 7.01 (d, 1H, J=7.9 Hz), 6.99 (d, 1H, J=3.3 Hz), 6.73 (d, 1H, J=2.1 Hz), 6.65 (dd, 1H, J=7.9, 0.8 Hz), 6.51 (d, 1H, J=3.3 Hz), 3.93 (s, 2H), 3.89 (s, 3H), 2.49 (s, 3H), 2.39 (br s, 1H); APCI m/z 301.1 (M+1).

### EXAMPLE 138

### [3-(4-Chlorophenoxy)-4-methylaminomethylphenyl]-acetonitrile

¹H NMR (400 MHz, CDCl₃) δ 7.45 (d, 1H, J=7.9 Hz), 7.35-7.30 (m, 2H), 7.09 (dd, 1H, J=7.9, 1.7 Hz), 6.98-6.94 (m, 2H), 6.74 (d, 1H, J=1.7 Hz), 4.51 (br s, 1H), 3.93 (s, 2H), 3.66 (s, 2H), 2.51 (s, 3H); APCI m/z 287.2 (M+1).

### EXAMPLE 139

### 3-[3-(4-Chlorophenoxy)-4-methylaminomethylphenyl]propan-1-ol

¹H NMR (400 MHz, CDCl₃) δ 7.28-7.23 (m, 3H), 6.93 (dd, 1H, J=7.7, 1.2 Hz), 6.89-6.83 (m, 2H), 6.67 (d, 1H, J=1.2Hz), 3.75 (s, 2H), 3.57 (t, 2H, J=6.2Hz), 3.12 (br s, 2H), 2.58 (t, 2H, J=7.9 Hz), 2.40 (s, 3H), 1.79-1.72 (m, 2H); ¹³C NMR (100 MHz, CDCl₃) δ 156.0, 154.9, 144.1, 131.2, 130.1, 128.5, 126.4, 124.5, 119.7, 119.2, 119.0, 62.0, 49.9, 35.3, 34.2, 32.0; APCI m/z 306.2 (M+1).

### EXAMPLE 140

### 3-[3-(4-Chlorophenoxy)-4-methylaminomethylphenyl]propionic acid ethyl ester

¹H NMR (400 MHz, CDCl₃) δ 7.33-7.25 (m, 3H), 6.99-6.95 (m, 2H), 6.93 (dd, 1H, J=7.9, 1.2 Hz), 6.60 (d, 1H, J=1.2 Hz), 6.38 (br s, 1H), 4.04 (q, 2H, J=7.1 Hz), 3.96 (s, 2H), 2.82 (t, 2H, J=7.7 Hz), 2.50 (s, 3H), 2.49 (t, 2H, J=7.7 Hz), 1.18 (t, 3H, J=7.1 Hz); ¹³C NMR (100 MHz, CDCl₃) δ 172.7, 156.0, 154.6, 144.3, 132.2, 130.3, 129.6, 124.0, 121.0, 117.4, 60.8, 48.6, 35.6, 33.6, 30.8, 14.4; APCI m/z 348.2 (M+1).

### EXAMPLE 141

### [3-(4-Chloro-2,3-dimethylphenoxy)-4-methylaminomethylphenyl]-acetonitrile

¹H NMR (400 MHz, CDCl₃) δ 7.45 (d, 1H, J=7.9 Hz), 7.20 (d, 1H, J=8.7 Hz), 7.04 (dd, 1H, J=7.9, 1.7 Hz), 6.73 (d, 1H, J=8.7 Hz), 6.47 (d, 1H, J=1.3 Hz), 4.86 (br s, 1H), 4.03 (s, 2H), 3.61 (s, 2H), 2.55 (s, 3H), 2.38 (s, 3H), 2.15 (s, 3H); APCI m/z 315.2 (M+1).

### EXAMPLE 142

### [4-Chloro-2-(1H-indazol-5-yloxy)benzyl]methylamine

¹H NMR (400 MHz, CDCl₃) δ 8.01 (d, 1H, J=0.8 Hz), 7.43 (d, 1H, J=9.1 Hz), 7.31 (d, 1H, J=8.3 Hz), 7.27 (d, 1H, 8.3 Hz), 7.07 (dd, 1H, J=8.7, 2.1 Hz), 7.02 (dd, 1H, J=8.1, 1.9 Hz), 6.71 (d, 1H, J=1.7 Hz), 3.90 (s, 2H), 2.51 (s, 3H); ¹³C NMR (100 MHz, CDCl₃) δ 157.4, 150.3, 137.8, 134.6, 133.9, 131.6, 128.1, 123.8, 123.1, 120.9, 117.5, 111.7, 110.0, 50.4, 35.8; APCI m/z 288.3 (M+1).

### EXAMPLE 143

### [4-Chloro-2-(1-methyl-1H-indazol-5-yloxy)benzyl]methylamine

¹H NMR (400 MHz, CDCl₃) δ 7.91 (s, 1H), 7.39 (d, 1H, J=9.1 Hz), 7.31 (d, 1H, J=7.9 Hz), 7.27 (d, 1H, J=2.1 Hz), 7.13 (dd, 1H, J=9.1, 2.1 Hz), 7.02 (dd, 1H, J=7.9, 2.1 Hz), 6.69 (d, 1H, J=2.1 Hz), 4.08 (s, 3H), 3.84 (s, 2H), 2.45 (s, 3H); ¹³C NMR (100 MHz, CDCl₃) δ 157.3, 150.3, 137.5, 133.7, 132.6, 131.4, 128.6, 124.6, 123.1, 120.5, 117.5, 110.7, 110.1, 50.4, 36.0; APCI m/z 302.2 (M+1).

### EXAMPLE 144

### [2-(4-Chloro-2,3-dimethylphenoxy)-4-oxazol-5-yl-benzyl]methylamine

¹H NMR (400 MHz, CDCl₃) δ 7.82 (s, 1H), 7.45 (d, 1H, J=7.9 Hz), 7.33 (dd, 1H, J=7.9, 1.7 Hz), 7.22 (s, 1H), 7.17 (d, 1H, J=8.7 Hz), 6.86 (d, 1H, J=1.7 Hz), 6.66 (d, 1H, J=8.7 Hz), 3.88 (s, 2H), 3.04 (br s, 1H), 2.48 (s, 3H), 2.38 (s, 3H), 2.21 (s, 3H); ¹³C NMR (100 MHz, CDCl₃) δ 156.3, 152.7, 151.0, 150.7, 136.9, 131.3, 130.2, 129.6, 128.5, 127.5, 122.1, 119.2, 117.7, 112.4, 50.3, 35.8, 17.2, 13.5; APCI m/z 343.1 (M+1).

### EXAMPLE 145

### [2-(4-Chloro-2,3-dimethylphenoxy)-4-(4-ethyl-oxazol-5-yl)benzyl]methylamine

¹H NMR (400 MHz, CDCl₃) δ 7.73 (s, 1H), 7.48 (d, 1H, J=7.9 Hz), 7.24 (dd, 1H, J=7.9, 1.7 Hz), 7.19 (d, 1H, J=8.7 Hz), 6.76 (d, 1H, J=8.7 Hz), 6.71 (s, 1H), 5.75 (br s, 1H), 4.06 (s, 2H), 2.61-2.55 (m, 5H), 2.35 (s, 3H), 2.15 (s, 3H), 1.13 (t, 3H, J=7.5 Hz); APCI m/z 371.2 (M+1).

### A1 Preparations

### Preparation 1

### 4-Chloro-2,5-difluoro-benzaldehyde

4-Chloro-2,5-difluoro-benzoic acid (25.0 g, 129.84 mmol), H₂SO₄ (3.46 mL, 64.92 mmol) and EtOH (300 mL) were combined and heated at reflux for 72 h. The solution was cooled, concentrated, diluted with H₂O (1.0 L), and extracted with EtOAc (3×500 mL). The combined organic extracts were washed with saturated NaHCO₃ (3×200 mL), H₂O (200 mL), dried (MgSO₄), filtered, and concentrated to provide 28.17 g of 4-chloro-2,5-difluoro-benzoic acid ethyl ester as a golden oil.

4-Chloro-2,5-difluoro-benzoic acid ethyl ester (28.17 g, 127.69 mmol) was dissolved in EtOH (900 mL), cooled in an ice bath to 0° C., and NaBH₄ (14.49 g, 383.08 mmol) was added portion wise over 20 min. Reaction was allowed to warm to r.t. and stirred for 15 h. The mixture was carefully diluted with H₂O (1.0 L) and extracted with EtOAc (3×400 mL). The combined organic extracts were washed with H₂O (2×300 mL), dried (MgSO₄), filtered, and concentrated to provide 22.10 g of (4-chloro-2,5-difluorophenyl)-methane as a pale yellow oil.

(4-Chloro-2,5-difluorophenyl)-methane (22.10 g, 123.76 mmol) was dissolved in CHCl₃ (1.0 L) and pyridinium chlorochromate (53.36 g, 247.53 mmol) was added portion wise over 30 min and the reaction was stirred for 24 h. Decanted liquid away from black tar, concentrated, and chromatographed (eluted with 5% EtOAc in hexanes) to provide 19.02 g of 4-chloro-2,5-difluoro-benzaldehyde as an off-white solid.

### Preparation 2

### 2-Fluoro-4-(1-hydroxypropyl)benzaldehyde

4-Bromo-2-fluoro-benzaldehyde (100 g, 490.63 mmol) and p-toluenesulfonic acid monohydrate (9.33 g, 49.06 mmol) were combined in MeOH (2.0 L) and heated at reflux for 14 h. The solution was cooled, diluted with 1N Na₂CO₃ (55.0 mL) and evaporated to remove the MeOH. Mixture was diluted with H₂O (500 mL) and extracted with EtOAc (3×600 mL). The combined organic extracts were washed with brine (2×500 mL), dried (Na₂SO₄), filtered, concentrated and distilled under high vacuum at 0.25 mm Hg with fractions collected between 90-103° C. to provide 116.20 g of 4-bromo-1-(dimethoxymethyl)-2-fluorobenzene as a clear oil.

4-Bromo-1-(dimethoxymethyl)-2-fluorobenzene (87.0 g, 349.27 mmol) was dissolved in THF (700 mL) and cooled to −78° C. n-Butyl lithium (185.42 mL of a 2.5 M soln in hexanes, 461.04 mmol) was rapidly added and immediately followed by addition of DMF (40.40 mL, 523.91 mmol). Reaction was stirred in the cold for 30 min then allowed to warm to rt and stirred for 1 h. The reaction solution was diluted with H₂O (500 mL) and extracted with Et₂O (3×500 mL). The combined organic extracts were washed with brine (3×200 mL), dried (Na₂SO₄), filtered, concentrated and chromatographed (eluted with 10% EtOAc in hexanes) to provide 45.90 g of 4-(dimethoxymethyl)-3-fluorobenzaldehyde as a clear oil.

4-(Dimethoxymethyl)-3-fluorobenzaldehyde (5.97 g, 30.12 mmol) was dissolved in Et₂O (50 mL) cooled to 0° C. and ethyl magnesium bromide (12.1 mL of a 3.0 M solution in Et₂O, 36.15 mmol) was added dropwise. The reaction mixture stirred in the cold for 1 h. then allowed to warm to r.t. and stirred for 15 h. Reaction was diluted with H₂O (400 mL) and extracted with EtOAc (3×400 mL). The combined organic extracts were washed with brine (400 mL), dried (Na₂SO₄), filtered, concentrated and chromatographed (eluted with 1% MeOH in CHCl₃) to provide 4.80 g of 1-[4-(dimethoxymethyl)-3-fluorophenyl]propan-1-ol as a clear oil.

1-[4-(Dimethoxymethyl)-3-fluorophenyl]propan-1-ol (4.8 g, 21.02 mmol) was dissolved in THF (80 mL), 1N HCl (21.02 mL) was added and reaction was stirred for 2 h. Reaction was diluted with H₂O (300 mL) and extracted with EtOAc (3×150 mL). The combined organic extracts were washed with H₂O (300 mL), brine (300 mL), dried (MgSO₄), filtered, concentrated to provide 3.77 g of 2-fluoro-4-(1-hydroxypropyl)benzaldehyde, Preparation 2, as a clear oil.

### Preparation 3

### 2,5-Difluoro-4-methyl-benzaldehyde

Larson, Eric Robert; Noe, Mark Carl; Gant, Thomas George. WO9962890 A1 19991209.

### Preparation 4

### 2.5-Difluoro-4-methoxy-benzaldehyde

A mixture of 4-bromo-2,6-difluoroanisole (4.46 g, 20.0 mmol), toluene (200 mL), and THF (10 mL) was cooled to −78° C. To the mixture was added n-BuLi (8.00 mL of a 2.5 M solution in hexanes, 20 mmol). After stirring at −78° C. for 1 h, DMF (3.10 ml, 40.0 mmol) was added. The mixture was stirred at −78° C. for 2 h and then H₂O (100 mL) was added. After warming to rt, the mixture was extracted with EtOAc (2×75 mL). The combined extracts were dried (MgSO₄), filtered and concentrated to provide 3.00 g of 2,5-difluoro-4-methoxy-benzaldehyde as a solid. ¹H NMR (400 MHz, CDCl₃): δ 10.18 (d, 1, J=2.9), 7.54 (dd, 1, J=10.6, 6.4), 6.72 (dd, 1, J=11.4, 6.4), 3.95 (s, 3).

### B1 Preparations

### Preparation 5

### 2,3-Dihydrobenzo[b]thiophen-5-ol

See Synth. Commun. 1991, 21, 959-964.

B1 Preparations 6-10 were prepared according to procedures outlined in patent WO 2002018333.

### Preparation 6

### 2,3-Dihydrobenzo[b]thiophen-6-ol

### Preparation 7

### 1,3-Dihydrobenzo[c]thiophen-5-ol

### Preparation 8

### 1,3-Dihydro-isobenzofuran-5-ol

### Preparation 9

### 2,3-Dihydrobenzo[1,4]oxathiin-7-ol

### Preparation 10

### 2,3-Dihydrobenzo[1,4]oxathiin-6-ol

### Preparation 11

### 2,3-Dihydrobenzofuran-5-ol

The preparation of 2,3-dihydrobenzofuran-5-ol was previously reported. MS (M⁺)=136. ¹H NMR consistent with literature. Hammond, M. L.; Kopka, I. E.; Zambias, R. A.; Caldwell, C. G.; Boger, J.; Baker, F.; Bach, T.; Luell, S.; MacIntyre, D. E. J. Med. Chem. 1989, 32, 1006-20. Pearlman, W. M. Tetrahedron Lett. 1967, 17, 1663. Selander, H.; Nilsson, J. L. G. Acta Chem. Scand. 1972, 26, 2433.

### Preparation 12

### 2,3-Dihydrobenzofuran-6-ol

To a mixture of 6-hydroxy-benzofuran-3-one (4.50 g, 30.0 mmol) in EtOAc (25 mL) at rt was added AcCl (8.98 mL, 126 mmol). The mixture was heated at rflux for 2 d, cooled, poured into ice/water, and extracted with EtOAc (2×100 mL). The extracts were combined, washed with sat. NaHCO₃ (100 mL), dried (MgSO₄), concentrated, and chromatographed (adsorbed onto silica; eluted with 20% EtOAc in hexanes) to provide 5.20 g of acetic acid 6-acetoxy-benzofuran-3-yl ester as a light yellow solid. ¹H NMR (400 MHz, DMSO-D₆): δ 8.21 (s, 1), 7.57 (d, 1, J=8.5), 7.46 (d, 1, J=2.1), 7.08 (dd, 1, J=8.5, 2.1) 2.36 (s, 3), 2.28 (s, 3).

To a solution of acetic acid 6-acetoxy-benzofuran-3-yl ester (600 mg, 2.56 mmol) in AcOH (35 mL) at rt under a N₂ atmosphere in a Parr bottle was added 5% Pd/C (300 mg). The mixture was placed under a H₂ atmosphere (10 psi) on a Parr shaker at 65° C. for 60 min. The mixture was cooled and filtered through a plug of Celite™, rinsing with EtOH. The filtrate was concentrated and the resulting yellow gum was dissolved in EtOAc (50 mL), washed with sat. NaHCO₃, dried (MgSO₄), filtered, and concentrated to provide 270 mg of light yellow solid. To a portion of the solid (250 mg) was added 1 M NaOH (5 mL) and the slurry was refluxed for 1H, cooled, acidified with 1M HCl (10 mL), and extracted with EtOAc (2×50 mL). The combined extracts were washed with brine, dried (MgSO₄), filtered, concentrated, and chromatographed (loaded with CH₂Cl₂; eluted with 20% EtOAc in hexanes) to provide 170 mg of 2,3-dihydrobenzofuran-6-ol as a light yellow gum. ¹H NMR (400 MHz, CDCl₃): δ 6.98-7.02 (m, 1), 6.33-6.37 (m, 2), 6.13 (s, 1), 4.57 (t, 2, J=8.7), 3.11 (t, 2, J=8.6). ¹³C NMR (100 MHz, CDCl₃): δ 161.1, 156.2, 125.4, 119.1, 107.7, 98.0, 72.4, 29.2. A similar preparation was previously reported: Horning, E. C.; Reisner, D. B. J. Am. Chem. Soc. 1948, 70, 3619-20.

### Preparation 13

### Benzofuran-7-ol

To 7-methoxy benzofuran (6.3 g, 42.5 mmol) in a 0.5 M solution of CH₂Cl₂ at −78° C. is added tetrabutylammonium iodide (18.9 g, 51 mmol). To this stirred solution, BCl₃ (100 mL of a 1.0 M solution in CH₂Cl₂, 100 mmol) was added dropwise via addition funnel. The reaction mixture was stirred at −78° C. for 6 h. Water is added dropwise very slowly to quench. The resulting mixture was stirred overnight at rt, basified with 6 N NaOH (pH=10), stirred for 1 h, neutralized with 2 N HCl (pH=7), and extracted with CH₂Cl₂ (5×). The combined extracts were dried (MgSO₄), filtered, concentrated, and chromatographed (10% EtOAc in hexanes) to provide 5.3 grams of benzofuran-7-ol as an oil. ¹H NMR (400 MHz, CDCl₃): δ 7.61 (d, 1, J=2.1), 7.19 (d, 1, J=7.7), 7.13 (t, 1, J=7.7), 6.88 (d, 1, J=7.7), 6.79 (d, 1, J=2.1), 6.04 (bs, 1). Previously prepared but no data reported: Musser, J. H.; Chakraborty, U.; Bailey, K.; Sciortino, S.; Whyzmuzis, C.; Amin, D.; Sutherland, C. A. J. Med. Chem. 1987, 30, 62-67.

### Preparation 14

### 2,3-Dihydrobenzofuran-7-ol

To a Parr flask charged with benzofuran-7-ol (1.12 g, 8.35 mmol) and EtOH (38 mL) was added 10% Pd/C (1.12 g). The Parr flask was placed under an H₂ atmosphere (50 psi) and shaken for 20 h at rt and then purged with N₂, filtered through a plug of Celite™, concentrated, and chromatographed (20% EtOAc in hexanes) to provide 0.7 g of 2,3-dihydrobenzofuran-7-ol as an oil. ¹H NMR (400 MHz, CDCl₃): δ 6.74-6.79 (m, 3), 5.38-5.45 (m, 1), 4.63 (t, 2, J=8.7), 3.25 (t, 2, J=8.7).

### Preparation 15

### Benzofuran-5-ol

To a mixture of 5-methoxybenzofuran (10.0 g, 67.5 mmol) and tetrabutylammonium iodide (31.2 g, 84.4 mmol) in CH₂Cl₂ (100 mL) at −78° C. was added BCl₃ (169 mL of a 1 M solution in CH₂Cl₂, 169 mmol) dropwise over 1 h. The reaction mixture was stirred for 1 h, warmed to rt, stirred for 14 h, poured slowly into sat. NaHCO₃ and ice, and extracted with CH₂Cl₂ (2×). The combined extracts were dried (MgSO₄), filtered, concentrated, and chromatographed (20% EtOAc in hexanes) to provide 9.16 g of benzofuran-5-ol as an orange solid. ¹H NMR (400 MHz, CDCl₃): δ 7.57 (d, 1, J=2.1), 7.34 (d, 1, J=8.7), 7.03 (d, 1, J=2.7), 6.84 (dd, 1, J=8.8, 2.6), 6.64 (dd, 1, J=2.2, 0.9), 6.20 (s, 1). Previously synthesized but data are not provided. Rene, L.; Royer, R. Bull. Soc. Chim. France, 1973, 7-8 (Pt. 2), 2355-6. The TBAl/BCl₃ procedural reference is Brooks, P. R.; Wirtz, M. C.; Vetelino, M. G.; Rescek, D. M.; Woodworth, G. F.; Morgan, B. P.; Coe, J. W. J. Org. Chem. 1999, 64, 9719-9721.

### Preparation 16

### 4-Methyl-2,3-dihydrobenzofuran-5-ol

To a solution of benzofuran-5-ol (9.10 g, 67.9 mmol) in MeOH (1.2 L) at −30° C. was added a solution of Br₂ (3.48 mL, 67.9 mmol) in MeOH (68 mL) dropwise via addition funnel over 30 min. After 1 h, sat. NaHCO₃ (150 mL) was added to the reaction mixture, which was then warmed to rt and concentrated. The resulting mixture was diluted with EtOAc, filtered to remove solids, then washed with sat. NaHCO₃ and brine, dried (MgSO₄), filtered, concentrated, and chromatographed (10% EtOAc in hexanes) to provide 10.6 g of 4-bromo-benzofuran-5-ol as a yellow solid. ¹H NMR (400 MHz, CDCl₃): δ 7.64 (d, 1, J=2.3), 7.36 (dd, 1, J=8.9, 0.8), 7.00 (d, 1, J=8.9), 6.72 (dd, 1, J=2.1, 0.8), 5.34 (bs, 1).

A mixture of 4-bromo-benzofuran-5-ol (7.90 g, 37.1 mmol), K₂CO₃ (15.4 g, 111 mmol), Nal (278 mg, 1.85 mmol), and BnBr (4.41 mL, 37.1 mmol) in acetone (250 mL) was refluxed for 14 h. The resulting solution was filtered, concentrated, dissolved in EtOAc, and washed with H₂O, 1 N NaOH, and brine. The EtOAc layer was dried (MgSO₄), filtered, concentrated and chromatographed (5% EtOAc in hexanes) to provide 9.74 g of 5-benzyloxy-4-bromo-benzofuran as a yellow gum. ¹H NMR (400 MHz, CDCl₃): δ 7.65 (d, 1, J=2.1), 7.51-7.54 (m, 1), 7.32-7.47 (m, 5), 6.98 (d, 1, J=8.9), 6.82 (dd, 1, J=2.1, 0.8), 5.18 (s, 2).

To a solution of 5-benzyloxy-4-bromo-benzofuran (9.70 g, 32.0 mmol) and Mel (9.96 mL, 160 mmol) in TBME (320 mL) at 0° C. was added n-BuLi (17.9 mL of a 2.5 M solution in hexanes, 44.8 mmol) dropwise over 5 min. After 1 hour, 3 M NH₄Cl (200 mL) was added slowly and the reaction mixture was warmed to rt and continued stirring for 14 h. H₂O was added to the resulting mixture, which was extracted with EtOAc (2×). The combined extracts were washed with brine, dried (MgSO₄), filtered, concentrated and chromatographed (5% EtOAc in hexanes) to provide 7.40 g of 5-benzyloxy-4-methyl-benzofuran as a yellow oil. ¹H NMR (400 MHz, CDCl₃): δ 7.60 (d, 1, J=2.3), 7.33-7.55 (m, 5), 7.29 (d, 1, J=8.7), 6.97 (d, 1, J=8.9), 6.77 (dd, 1, J=2.3, 0.8), 5.11 (s, 2), 2.46 (s, 3).

A mixture of 5-benzyloxy-4-methyl-benzofuran (7.40 g, 24.4 mmol) and 10% Pd/C (7.50 g) in EtOH under N₂ was placed under an H₂ atmosphere (50 psi) on a Parr shaker for 14 h. The Parr bottle was purged with N₂, filtered through a plug of Celite™, concentrated, and chromatographed (10% EtOAc in hexanes) to provide 2.25 g of 4-methyl-2,3-dihydrobenzofuran-5-ol as a white solid. ¹H NMR (400 MHz, CDCl₃): δ 6.53 (d, 1, J=8.3), 6.48 (d, 1, J=8.5), 4.55 (t, 2, J=8.6), 4.40 (bs, 1), 3.11 (t, 2, J=8.6), 2.16 (s, 3). ¹H NMR data consistent with literature: Hammond, M. L. et al. J. Med. Chem. 1989, 32,1006-20.

### Preparation 17

### 4-Chloro-benzofuran-5-ol

To benzofuran-5-ol (5.0 g, 37 mmol) in CH₂Cl₂ (74 mL) at rt under N₂ was added thionyl chloride (2.75 mL, 33 mmol) followed by addition of Et₂O (3.54 mL, 33 mmol). After 10 min, additional thionyl chloride (0.31 mL, 3.7 mmol) and Et₂O (0.40 mL, 3.7 mmol) were added. The reaction mixture was stirred for another 10 min, concentrated, and chromatographed (10% EtOAc in hexanes) to provide 4.03 g of 4-chloro-benzofuran-5-ol. GC-MS 168 at 1.71 min. ¹H NMR (400 MHz, CDCl₃): δ 7.60 (d, 1, J=2.3), 7.30 (d, 1, J=8.7), 6.97 (d, 1, J=8.9), 6.76 (d, 1, J=2.1), 5.37 (bs, 1).

### Preparation 18

### 4-Chloro-2,3-dihydrobenzofuran-5-ol

A mixture of 4-chloro-benzofuran-5-ol (84.3 mg, 0.500 mmol), 5% Pt/C (85 mg) and MeOH (10 mL) were placed under H₂ atmosphere (50 psi) at rt for 19 h. The Parr bottle was purged with N₂ and more 5% Pt/C (340 mg) was added. The Parr bottle was placed back under H₂ atmosphere (50 psi) for 20 h, purged again with N₂, charged with more MeOH (10 mL) and 5% Pt/C (250 mg), and placed back under H₂ atmosphere (50 psi) for 36 h. After the final N₂ purge, the reaction mixture was filtered through a plug of Celite™ and concentrated. The crude material was purified by chromatography (1:1 EtOAc/hexanes) to provide 54 mg of 4-chloro-2,3-dihydrobenzofuran-5-ol as a light tan gum. MS (M⁺)=170, 172. ¹H NMR (400 MHz, CDCl₃): δ 6.70 (d, 1, J=8.5), 6.52 (d, 1, J=8.5), 4.52 (t, 2, J=8.7), 3.16 (t, 2, J=8.7).

### Preparation 19

### 1H-indazol-5-ol

Fuming sulfuric acid (1.5 mL) was added to a slurry of 1H-indazol-5-ylamine (750 mg, 5.6 mmol) in water (3.5 mL). This mixture was heated to completely dissolve the starting amine and was then capped and placed in the microwave at 180 C for 15 hours. The reaction mixture was poured onto 50 mL of ice/water. Solid sodium hydroxide was added until pH>10. This mixture was extracted with ethyl acetate (4×20 mL) and the combined organic phases were washed with brine, dried over sodium sulfate, filtered and concentrated to give 682 mg of the title compound as a white solid.

### Preparation 20

### 1-Methyl-1H-indol-5-ol and 1-Methyl-2,3-dihydro-1H-indol-5-ol

Sodium hydride (60% in oil, 2.0 g, 52 mmol) was added to a solution of 5-benzyloxy-1H-indole (10 g, 45 mmol) in DMF (60 mL) at 0 C. After 1 h, methyl iodide (3.3 ml, 52 mmol) was added. After 90 min, the reaction mixture was poured into ice/water, the resulting solid was collected by filtration and air dried to give 6.0 g of 5-benzyloxy-1-methyl-1H-indole. This material was dissolved in ethyl acetate (125 mL) and acetic acid (25 mL). Next add 5% Pd/C and shake under an atmosphere of hydrogen at 45 psi for 66 h at rt. The resulting mixture was filtered through a pad of Celite™ and concentrated. The residue was purified by flash chromatography eluting with hexanes, then 5/1 hexanes/ethyl acetate to afford 2.4 g of 1-methyl-1H-indole-5-ol and 0.55 g of 1-methyl-2,3-dihydro-1H-indol-5-ol.

### Preparation 21

### 1,3-Dimethyl-1H-indol-5-ol

The title compound was prepared using the methods analogous to those described in Preparation 20 starting with 5-benzyloxy-3-methyl-1H-indole (prepared as described by Marino, J. P., et al J. Amer. Chem. Soc. 1992, 114, 5566).

### Preparation 22

### 5-Hydroxy-3-methyl-indole-1-carboxylic acid tert-butyl ester

5-benzyloxy-3-methyl-1H-indole (1.0 g, 4.2 mmol, prepared as described by Marino, J. P., et al. J. Amer. Chem. Soc. 1992, 114, 5566) was dissolved in acetonitrile (15 mL) and DMAP (50 mg, 0.4 mmol), then (Boc)₂O (1.3 g, 6.0 mmol) were added and the reaction mixture stirred at rt for 18 h. Dilute the mixture with EtOAc and wash with water then brine. The organic layer was concentrated to and oil to give 5-benzyloxy-3-methyl-indole-1-carboxylic acid tert-butyl ester (1.2 g) which was converted to the title compound using an analogous method to that described in Preparation 20.

### Preparation 23

### 4-Fluoro-1-methyl-1H-indol-5-ol

4-Fluoro-5-methoxy-1H-indole (see: Laban, U. et al Bioorg. Med. Chem. Lett. 2001, 11, 793) was converted to 4-fluoro-5-methoxy-1-methyl-1H-indole using the method described in Preparation 20. This material (0.8 g, 4.5 mmol) was dissolved in methylene chloride (50 mL) and 1 M boron tribromide (in CH₂Cl₂, 13.5 ml, 13.5 mmol) was added at 0 C. After 2 h the mixture was quenched with sat NaHCO₃ until the mixture was basic. The organic phase was concentrated to give the title compound.

### Preparation 24

### 1,3-Dimethyl-2,3-dihydro-1H-indol-5-ol

1,3-Dimethyl-1H-indol-5-ol (preparation 21) was subjected to hydogenolysis conditions analogous to those described in Preparation 20 to provide the title compound.

### Preparation 25

### 1-Methyl-1H-benzoimidazol-5-ol

4-Methoxy-2-nitrophenylamine (5.0 g, 30 mmol) was dissolved in DMF (50 mL) and cooled to 10 C. NaH (63% in oil, 1.3 g, 33 mmol) was added portionwise and the mixture stirred for 30 min. Methyl iodide (2.1 mL, 33 mmol) was added and after 30 min the mixture was quenched with water. The resulting solid was collected by filtration and diluted with ethyl acetate (200 mL). 5% Pd/C (1.0 g) was added and the mixture was shaken under an atmosphere of hydrogen at 50 psi for 3 h. The reaction mixture was filtered through a pad of Celite™ and concentrated to give 4-Methoxy-N¹-methyl-benzene-1,2-diamine (1.0 g). This material was heated in formic acid (2 mL) at 100 C for 90 min. The mixture was cooled to rt, diluted with EtOAc and quenched with sat. NaHCO₃ until basic. The organic phase was collected, dried and concentrated to give 5-methoxy-1-methyl-1H-benzoimidazole (0.9 g) which was converted to the title compounds using the method described in Preparation 23.

### Preparation 26

### 1,4-Dimethyl-1H-indol-5-ol

Pyrrolidine (3.8 mL, 45 mmol) and DMF-DMA (4.3 mL, 32 mmol) were added to a solution of 1-methoxy-2,3-dimethyl-4-nitro-benzene (5.0 g, 28 mmol) in DMF (25 mL) and the mixture was heated at 135 C for 2 h. After cooling to rt the mixture was partitioned between EtOAc and water. The organic phase was collected, dried and concentrated to give 1-[2-(3-methoxy-2-methyl-6-nitrophenyl)-vinyl]-pyrrolidine (˜5 g). This material was dissolved in EtOAc (100 mL) and 10% Pd/C (0.5 g) was added and the mixture was shaken under an atmosphere of hydrogen at 50 psi at rt for 3 h. The resulting mixture was filtered through a pad of Celite™ and purified by chromatography (elution with 10% EtOAc/hexanes) to afford 5-methoxy-4-methyl-1H-indole (2.2 g). This material was converted to the title compound using the procedures described in Preparation 20 and Preparation 23.

### Preparation 27

### 1,4-Dimethyl-2,3-dihydro-1H-indol-5-ol

The title compound was prepared from 1,4-dimethyl-1H-indol-5-ol (preparation 26) using the method described in Preparation 20.

### Preparation 28

### 1-Methyl-1,2,3,4-tetrahydro-quinolin-6-ol

10% Pd/C (600 mg) was added to a solution of 6-methoxy-quinoline (6.0 g, 38 mmol) in ethanol (100 mL). The resulting mixture was shaken under an atmosphere of hydrogen at 45 psi at rt for 6 days. The mixture was filtered through a pad of Celite™ to remove the catalyst and concentrated to afford 6-methoxy-1,2,3,4-tetrahydro-quinoline (6.0 g). This material (1.0 g, 6 mmol) was dissolved in acetonitrile (50 mL) and an aqueous solution of formaldehyde (37%, 4.5 mL, 60 mmol), acetic acid (1 mL) and sodium triacetoxyborohydride (3.0 g, 15 mmol) were added and the mixture was stirred for 2 h at rt. The acetonitrile was evaporated and the residue was partitioned between EtOAc and water. This mixture was made basic with saturated NaHCO₃. The organic phase was collected, dried and concentrated to afford 6-methoxy-1-methyl-1,2,3,4-tetrahydro-quinoline (1.0 g). This material was dissolved in acetic acid (10 mL) and a 48% HBr solution (5.0 mL) was added and the resulting mixture was stirred at 100 C for 3 days. The solvent was removed by evaporation and the resulting residue was partitioned between EtOAc and water. Saturated aqueous NaHCO₃ was added until the pH=7.5. The organic phase was collected, dried and concentrated and the crude residue was purified by chromatography (elution with 10:1 hexanes/EtOAc) to afford the title compound (0.54 g).

### Preparation 29

### 6-Hydroxy-4H-benzo[1,4]oxazin-3-one

Lanthanum nitrate hexahydrate (1.6 g, 5 mmol) and sodium nitrate (4.25 g, 50 mmol) were added to a solution of 4-benzyloxy-phenol (10 g, 50 mmol) in 6 N HCl (110 mL) and ether (400 mL). The mixture was stirred 20 h at rt. The mixture was filtered and the organic phase was collected, dried and concentrated. The crude residue was purified by chromatography (elution with 20:1 hexanes/EtOAc) to afford 4-benzyloxy-2-nitrophenol (6.2 g). Methyl bromoacetate (1.1 mL, 12 mmol), potassium carbonate (3.3 g, 24 mmol) and sodium iodide (10 mg) were added to a solution of this material (2.5 g, 10 mmol) in acetone (50 mL). The resulting mixture was stirred at 50 C for 4 h, then at rt for 18 h. The solvent was evaporated and the residue was partitioned between EtOAc and water. The organic phase was collected, dried and concentrated to a dark oil that slowly crystallized. The resulting solid was triturated with isopropyl ether and the resulting solid was collected to afford (4-benzyloxy-2-nitrophenoxy)-acetic acid methyl ester (2.7 g). This material was dissolved methanol (150 mL) and ammonium chloride (4.5 g, 85 mmol) and zinc (3.3 g, 51 mmol) were added. This mixture was stirred for 1 h at rt and then heated to 55 C for 1.5 h. The resulting mixture was filtered and concentrated and the residue was partitioned between EtOAc and water. The organic phase was collected, dried and concentrated to afford 6-benzyloxy-4H-benzo[1,4]oxazin-3-one (1.8 g). This material was converted to the title compound using an method analogous to that described in Preparation 20 to remove the benzyl protecting group.

### Preparation 30

### 7-Hydroxy-4H-benzo[1,4]oxazin-3-one

Benzyl bromide (22 mL, 180 mmol), potassium carbonate (50 g, 360 mmol) and sodium iodide (1 g) were added to a solution of benzene-1,3-diol (20 g, 180 mmol) in acetone (200 mL) and the resulting mixture was stirred at rt for 2 days. The solvent was removed by filtration and the residue was partitioned between EtOAc and water. The organic phase was collected, dried and concentrated to an oil which eventually crystallized upon standing. The solid was purified by chromatography (elution with 10:1 hexanes/EtOAc then 3:1 hexanes/EtOAc) to afford 3-benzyloxy-phenol (15 g). This material was nitrated using the method described in Preparation 29 to afford 5-benzyloxy-2-nitrophenol (4.4 g) and 3-benzyloxy-4-nitrophenol (5.2 g). The 5-benzyloxy-2-nitrophenol was converted to the title compound using methods analogous to that described in Preparation 29.

### Preparation 31

### 1-Methyl-1H-indol-7-ol

Bromodiphenylmethane (8.9 g, 36 mmol), potassium carbonate (14 g, 100 mmol), and sodium iodide (500 mg) were added to a solution of 2-nitrophenol (5.0 g, 36 mmol) in acetone (100 mL). The resulting mixture was heated at 50 C for 24 h, filtered and concentrated. The residue was partitioned between EtOAc and water and the organic phase was collected, dried and concentrated. The crude residue was purified by chromatography (elution with 20:1 hexanes/EtOAc to 10:1 hexanes/EtOAc) to afford 2-nitro-diphenylmethyoxybenzene (7.5 g). This material (6.5 g, 21.3 mmol) was dissolved in THF (200 mL) and cooled to −70 C. Vinyl magnesium bromide (1M, 70 mL, 70 mmol) was slowly added and the mixture stirred 60 min at −70 C and was then allowed to warm to rt at which time it was poured into saturated aqueous ammonium chloride solution (200 mL). EtOAc was added and the layers were separated. The organic layer was washed with brine, dried, filtered and concentrated. The crude residue was triturated with 10:1 hexanes/EtOAc to afford 7-diphenylmethyloxy-1H-indole. This material was converted to the title compound using methods analogous to those described in Preparation 20.

### Preparation 32

### 1-Methyl-1H-indol-4-ol

The title compound was prepared from 4-benzyloxy-1H-indole according to the methods described in Preparation 20.

### Preparation 33

### 4-Hydroxy-indole-1-carboxylic acid tert-butyl ester

The title compound was prepared from 4-benzyloxy-1H-indole according to the methods described in Preparation 22.

### Preparation 34

### 6-Hydroxy-1H-quinolin-2-one

Acetic anhydride (0.94 mL, 10 mmol) was added to a solution of 6-methoxyquinoline N-oxide (1.75 g, 10 mmol, see: Dimsdale, M. J. J. Het. Chem. 1979, 16, 1209) in tertiary butanol (10 mL). The resulting mixture was heated at 80 C for 18 h then additional acetic anhydride (0.94 mL, 10 mmol) was added. The mixture was heated at 80 C for an additional 2 days. The reaction mixture cooled to rt and was diluted with EtOAc and water. The organic phase was collected and washed with sat. NaHCO₃, dried and concentrated to afford 6-methoxy-1H-quinolin-2-one (0.45 g). This material was converted to the title compound using the method described in Preparation 23.

### Preparation 35

### 2,3-Dihydrobenzofuran-4-ol

To a solution of 2′,6′-dihydroxyacetophenone (3.04 g, 20.0 mmol) in THF (40 mL) at −78° C. under N₂ atmosphere was added LiHMDS (66 mL of a 1.0 M soln in hexanes) over 15 min. The resulting yellow slurry was stirred at −78° C. for 1 h. TMSCl (12.2 mL, 120 mmol) was added quickly. After 10 min the reaction mixture was placed in a 0° C. bath, stirred for 1 h, and concentrated. The resulting material was diluted with hexanes (100 mL), filtered to remove solids, and concentrated. The yellow oil was dissolved in THF (100 mL) and cooled to 0° C. NBS (3.92 g, 22.0 mmol) was added and the reaction mixture was warmed to rt, stirred for 14 h and then refluxed for 2 h. After cooling the reaction mixture, 1 M NaOH (100 mL) was added and the mixture was heat at 65° C. for 14 h. After cooling, the mixture was concentrated to remove most of the THF, acidified with 6 M HCl (17 mL), and extracted with EtOAc (2×). The combined extracts were washed with brine, dried (MgSO₄), filtered, concentrated, and chromatographed (preabsorbed to SiO₂; 40% EtOAc in hexanes) to provide 1.80 g of 4-hydroxy-benzofuran-3-one as a pale orange solid. ¹H NMR (400 MHz, DMSO-D₆): δ 10.85 (bs, 1), 7.47 (t, 1, J=8.1), 6.60 (d, 1, J=8.1), 6.48 (d, 1, J=8.1), 4.68 (s, 2).

To a solution of 4-hydroxy-benzofuran-3-one (1.80 g, 12.0 mmol) in 1,2-dichloroethane (100 mL) at 0° C. was added Et₃N (13.7 mL, 98.5 mmol) followed by the slow addition of acetyl chloride (6.67 mL, 93.8 mmol). After 10 min, the slurry was warmed to rt over 1 h, poured into H₂O (200 mL), and extracted with CH₂Cl₂ (5×75 mL). The combined extracts were dried (MgSO₄), filtered, concentrated and chromatographed (10-20% EtOAc in hexanes) to provide 2.58 g of acetic acid 3-acetoxy-benzofuran-4-yl ester as a solid. MS (M⁺)=235. ¹H NMR (400 MHz, CDCl₃): δ 8.03 (s, 1), 7.36 (dd, 1, J=8.4, 0.9), 7.31 (d, 1, J=7.9), 6.96 (dd, 1, J=7.7, 0.8), 2.38 (s, 3), 2.32 (s, 3).

To a Parr flask charged with acetic acid 3-acetoxy-benzofuran-4-yl ester (2.58 g, 11.0 mmol) and glacial AcOH (100 mL) under N₂ was added 5% Pd/C (2.6 g of 50% wet). The Parr flask was placed under an H₂ atmosphere (10 psi), heated to 65° C., shaken for 4 h, and then purged with N₂. The resulting mixture was cooled, filtered through a plug of Celite™, and concentrated. The crude material was dissolved in MeOH (50 mL) and 6 M NaOH (18 mL) was added. The resulting dark solution was stirred overnight, concentrated, poured into 1 M HCl (150 mL), and extracted with CH₂Cl₂ (2×125 mL). The combined extracts were dried (Na₂SO₄), filtered, concentrated, and chromatographed (15-20% EtOAc in hexanes) to provide 875 mg of 2,3-dihydrobenzofuran-4-ol. MS (M⁺)=137. ¹H NMR (400 MHz, CDCl₃): δ 6.98 (t, 1, J=8.0), 6.41 (d, 1, J=8.1), 6.31 (d, 1, J=8.1), 4.60 (t, 2, J=8.7), 3.16 (t, 2, J=8.7). Previously prepared but data were not reported: Li, W-S. et al. Tetrahedron, Lett. 2002, 43, 1923-1925.

Radioligand binding assays were performed according to Pazos et al., Eur. J. Pharm., 1984, 106, 539-546, with some modifications. Frozen cell paste expressing 5-HT2A or 5-HT2C receptors was homogenized using a Brinkman Polytron model PT3000 (setting 15,000 rpm, 15 seconds) in 50 mM Tris HCl buffer pH 7.4 containing 2 mM MgCl2. The homogenate was centrifuged for ten minutes at 40,000 g, washed and recentrifuged. The 5-HT2A final pellet was resuspended in 50 mM Tris HCl buffer pH 7.4 at 37° C. and 5-HT2C final pellet was resuspended in 50 mM Tris HCl buffer pH 7.4 at 37° C. containing 4 mM CaCl2, 0.1% ascorbic acid and 100 μM pargyline. Incubations were initiated by the addition of tissue homogenate to wells of 96 well plates containing radioligand (5-HT2A: ³H-ketanserin, 0.7 nM final concentration; 5-HT2C: ³H-5-HT, 1 nM final concentration) and varying concentrations of test compound in a final volume of 250 μl. Non-specific binding was defined as the radioactivity remaining in the presence of a saturating concentration of cinanserin (5-HT2A assays) or mianserin (5-HT2C assays). Incubations were ended by rapid filtration (5-HT2A: 15 minute incubation at 37° C., 5-HT2C: 30 minute incubation at 37° C.) onto GF/B filtermats presoaked in 0.5% polyethylenimine, using a Skatron cell harvester (Molecular Devices) and washed with ice-cold 50 mM Tris buffer pH 7.4 at 4° C. Radioactivity was quantified by liquid scintillation counting (Betaplate, Wallac Instruments). The IC50 value (concentration at which 50% inhibition of specific binding occurs) was calculated by linear regression of the concentration-response data. Ki values were calculated according to Cheng & Prusoff, where Ki=IC50/(1+(L/Kd)), where L is the concentration of the radioligand used in the experiment and the Kd value is the dissociation constant for the radioligand (determined previously by saturation analysis).

In the following Examples, the term ‘active compound’ or ‘active ingredient’ refers to a suitable combination or individual element of a compound of Formula Ia, Ib, or Ic and an SSRI and/or NRI, or mixtures thereof and/or a pharmaceutically acceptable salt or solvate, according to the present invention.

(i) Tablet Compositions

The following compositions A and B can be prepared by wet granulation of ingredients (a) to (c) and (a) to (d) with a solution of povidone, followed by addition of the magnesium stearate and compression.

| | mg/tablet | mg/tablet |
|---|---|---|
| Composition A | | |
| (a) Active ingredient | 250 | 250 |
| (b) Lactose B.P. | 210 | 26 |
| (c) Sodium Starch Glycollate | 20 | 12 |
| (d) Povidone B.P. | 15 | 9 |
| (e) Magnesium Stearate | 5 | 3 |
| | 500 | 300 |
| Composition B | | |
| (a) Active ingredient | 250 | 250 |
| (b) Lactose | 150 | 150 |
| (c) Avicel PH 101 | 60 | 26 |
| (d) Sodium Starch Glycollate | 20 | 12 |
| (e) Povidone B.P. | 15 | 9 |
| (f) Magnesium Stearate | 5 | 3 |
| | 500 | 300 |
| Composition C | | |
| Active ingredient | 100 | |
| Lactose | 200 | |
| Starch | 50 | |
| Povidone | 5 | |
| Magnesium Stearate | 4 | |
| | 359 | |

The following compositions D and E can be prepared by direct compression of the admixed ingredients. The lactose used in formulation E is of the direct compression type.

| | | mg/tablet |
|---|---|---|
| Composition D | | |
| Active ingredient | | 250 |
| Magnesium Stearate | | 4 |
| Pregelatinised Starch NF15 | | 146 |
| | | 400 |
| Composition E | | |
| Active ingredient | | 250 |
| Magnesium Stearate | | 5 |
| Lactose | | 145 |
| Avicel | | 100 |
| | | 500 |
| Composition F | | |
| (Controlled release composition) | | |
| (a) | Active ingredient | 500 |
| (b) | Hydroxypropylmethylcellulose | 112 |
| | (Methocel K4M Premium) | |
| (c) | Lactose B.P. | 53 |
| (d) | Povidone B.P.C. | 28 |
| (e) | Magnesium Stearate | 7 |

700

The composition can be prepared by wet granulation of ingredients (a) to (c) with a solution of povidone, followed by addition of the magnesium stearate and compression.

Composition G (Enteric-Coated Tablet)

Enteric-coated tablets of Composition C can be prepared by coating the tablets with 25 mg/tablet of an enteric polymer such as cellulose acetate phthalate, polyvinylacetate phthalate, hydroxypropylmethyl-cellulose phthalate, or anionic polymers of methacrylic acid and methacrylic acid methyl ester (Eudragit L). Except for Eudragit L, these polymers should also include 10% (by weight of the quantity of polymer used) of a plasticizer to prevent membrane cracking during application or on storage. Suitable plasticizers include diethyl phthalate, tributyl citrate and triacetin.

Composition H (Enteric-Coated Controlled Release Tablet)

Enteric-coated tablets of Composition F can be prepared by coating the tablets with 50 mg/tablet of an enteric polymer such as cellulose acetate phthalate, polyvinylacetate phthalate, hydroxypropylmethyl-cellulose phthalate, or anionic polymers of methacrylic acid and methacrylic acid methyl ester (Eudgragit L). Except for Eudgragit L, these polymers should also include 10% (by weight of the quantity of polymer used) of a plasticizer to prevent membrane cracking during application or on storage. Suitable plasticizers include diethyl phthalate, tributyl citrate and triacetin.

(ii) Capsule Compositions

Composition A

Capsules can be prepared by admixing the ingredients of Composition D above and filling two-part hard gelatin capsules with the resulting mixture. Composition B (infra) may be prepared in a similar manner.

| | mg/capsule |
|---|---|
| Composition B | |
| (a) Active ingredient | 250 |
| (b) Lactose B.P. | 143 |
| (c) Sodium Starch Glycollate | 25 |
| (d) Magnesium Stearate | 2 |
| | 420 |
| Composition C | |
| (a) Active ingredient | 250 |
| (b) Macrogol 4000 BP | 350 |
| | 600 |

Capsules can be prepared by melting the Macrogol 4000 BP, dispersing the active ingredient in the melt and filling two-part hard gelatin capsules therewith.

Composition D

| | mg/capsule |
|---|---|
| Active ingredient | 250 |
| Lecithin | 100 |
| Arachis Oil | 100 |
| | 450 |

Capsules can be prepared by dispersing the active ingredient in the lecithin and arachis oil and filling soft, elastic gelatin capsules with the dispersion.

Composition E (Controlled release capsule)

| | mg/capsule |
|---|---|
| (a) Active ingredient | 250 |
| (b) Microcrystalline Cellulose | 125 |
| (c) Lactose BP | 125 |
| (d) Ethyl Cellulose | 13 |
| | 513 |

The controlled release capsule formulation can be prepared by extruding mixed ingredients (a) to (c) using an extruder, then spheronising and drying the extrudate. The dried pellets are coated with a release controlling membrane (d) and filled into two-part, hard gelatin capsules.

Composition F (Enteric capsule)

| | mg/capsule |
|---|---|
| (a) Active ingredient | 250 |
| (b) Microcrystalline Cellulose | 125 |
| (c) Lactose BP | 125 |
| (d) Cellulose Acetate Phthalate | 50 |
| (e) Diethyl Phthalat | 5 |
| | 555 |

The enteric capsule composition can be prepared by extruding mixed ingredients (a) to (c) using an extruder, then spheronising and drying the extrudate. The dried pellets are coated with an enteric membrane (d) containing a plasticizer (e) and filled into two-part, hard gelatin capsules.

Composition G (Enteric-Coated Controlled Release Capsule)

Enteric capsules of Composition E can be prepared by coating the controlled-release pellets with 50 mg/capsule of an enteric polymer such as cellulose acetate phthalate, polyvinylacetate phthalate, hydroxypropylmethylcellulose phthalate, or anionic polymers of methacrylic acid and methacrylic acid methyl ester (Eudragit L). Except for Eudragit L, these polymers should also include 10% (by weight of the quantity of polymer used) or a plasticizer to prevent membrane cracking during application or on storage. Suitable plasticizers include diethyl phthalate, tributyl citrate and triacetin.

(iii) Intravenous injection composition

| | | |
|---|---|---|
| Active ingredient | 0.200 | g |
| Sterile, pyrogen-free phosphate buffer (pH 9.0) to | 10 | ml |

The active ingredient is dissolved in most of the phosphate buffer at 35-40° C., then made up to volume and filtered through a sterile micropore filter into sterile 10 ml glass vials (Type 1) which are sealed with sterile closures and overseals.

(iv) Intramuscular injection composition

| | | |
|---|---|---|
| Active ingredient | 0.20 | g |
| Benzyl Alcohol | 0.10 | g |
| Glycofurol 75 | 1.45 | g |
| Water for Injection q.s. to | 3.00 | ml |

The active ingredient is dissolved in the glycofurol. The benzyl alcohol is then added and dissolved, and water added to 3 ml. The mixture is then filtered through a sterile micropore filter and sealed in sterile 3 ml glass vials (Type 1).

(v) Syrup composition

| | | |
|---|---|---|
| Active ingredient | 0.25 | g |
| Sorbitol Solution | 1.50 | g |
| Glycerol | 1.00 | g |
| Sodium Benzoate | 0.005 | g |
| Flavour | 0.0125 | ml |
| Purified Water q.s. to | 5.0 | ml |

The sodium benzoate is dissolved in a portion of the purified water and the sorbitol solution added. The active ingredient is added and dissolved. The resulting solution is mixed with the glycerol and then made up to the required volume with the purified water.

(vi) Suppository composition

| | mg/suppository |
|---|---|
| Active ingredient | 250 |
| Hard Fat, BP (Witepsol H15 - Dynamit NoBel) | 1770 |
| | 2020 |

One-fifth of the Witepsol H15 is melted in a steam-jacketed pan at 45° C. maximum. The active ingredient is sifted through a 200 lm sieve and added to the molten base with mixing, using a Silverson fitted with a cutting head, until a smooth dispersion is achieved. Maintaining the mixture at 45° C., the remaining Witepsol H15 is added to the suspension which is stirred to ensure a homogenous mix. The entire suspension is then passed through a 250 lm stainless steel screen and, with continuous stirring, allowed to cool to 40° C. At a temperature of 38-40° C., 2.02 g aliquots of the mixture are filled into suitable plastic moulds and the suppositories allowed to cool to room temperature.

(vii) Pessary composition

| | mg/pessary |
|---|---|
| Active ingredient (63\|m) | 250 |
| Anhydrous Dextrose | 380 |
| Potato Starch | 363 |
| Magnesium Stearate | 7 |
| | 1000 |

The above ingredients are mixed directly and pessaries prepared by compression of the resulting mixture.

(viii) Transdermal composition

| | | |
|---|---|---|
| Active ingredient | 200 | mg |
| Alcohol USP | 0.1 | ml |
| Hydroxyethyl cellulose | | |

The active ingredient and alcohol USP are gelled with hydroxyethyl cellulose and packed in a transdermal device with a surface area of 10 cm².

What is claimed is:

1. A 5HT2 antagonist having the formula Ia, Ib or Ic: 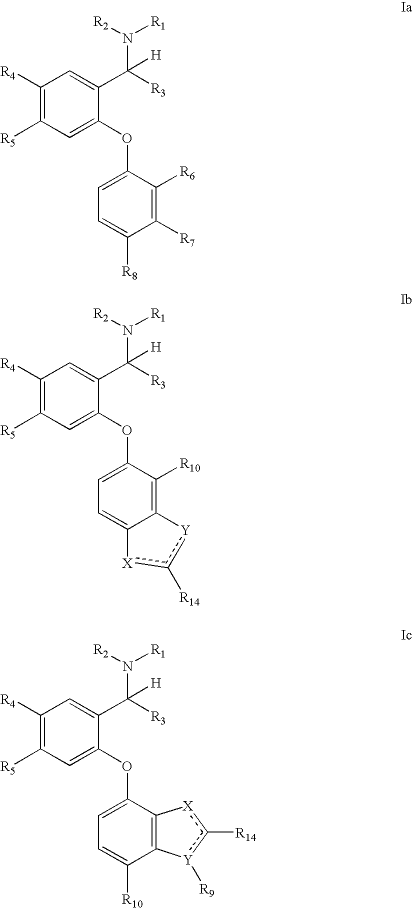
wherein X and Y are independently O, O(CH₂)ₙ, S, S(CH₂)ₙ, N, NR₁₈, NR₁₈N, NR₁₈(CH₂)ₙ, CR₁₈R₁₉(CH₂)ₙ or (CR₁₈R₁₉)ₖ, where R₁₈ and R₁₉ are independently H, straight chain or branched C₁-C₆ alkyl, CF₃, CN;
R₁, R₂ and R₃are, independently, H or CH₃;
R₄ is H, F, Cl or CH₃;
R₅ is F, Cl, Br, C₁-C₆ alkyl, (CH₂)ₙCN, (CH₂)ₙOH, (CH₂)ₙCO₂Et, C₁-C₆ cycloalkyl, oxazolyl or substituted oxazolyl, CR₁₁R₁₂—(CH₂)ₙCH₃, or S(O)ₘ—(CH₂)ₚCH₃;
R₆ is H, F, Cl, Br, O(CH₂)ᵣCH₃, C₁-C₆ alkyl, or CN;
R₇ is H, F, Cl, Br, C₁-C₆ alkyl, O—(CH₂)ₛCH₃, Cl, CN, N(R₁₃)(R₁₅), or OH;
R₈ is H, F, Cl, Br, C₁-C₆ alkyl, O—(C₁-C₆ alkyl), S—(C₁-C₆ alkyl), OH, NH—R₁₆, or S(O)ₜ—(C₁-C₆) alkyl;
R₉ is H, CH₃, OH, or, if Y is C, R₉ is ═O;
R₁₀ is H, Cl, F, Br, C₁-C₆ alkyl, O—(C₁-C₆ alkyl), S—(C₁-C₆ alkyl), OH, NH—R₁₇, or S(O)ᵤ—(C₁-C₆ alkyl); or, R₆ and R₇, or R₇ and R₈, or R₉ and R₁₀, together with the atoms to which they are attached, form a 5- to 8-membered ring containing one or more heteroatoms selected from the group consisting of N, O, and S;
R₁₁ and R₁₂ are, independently, H, OH, O—(C₁-C₆ alkyl), C₁-C₆ alkyl, S(O)ᵥ—(C₁-C₆ alkyl), CO—NH—(C₁-C₆ alkyl), O—(C₁-C₆ alkyl), (CH₂)ₙ—S(O)ₘ—(C₁-C₆ alkyl), or CO—NH-aryl;
R₁₃, R₁₅, R₁₆, and R₁₇ are, independently, H, or C₁-C₆ alkyl;
R₁₄ is H, CH₃, Cl, OH, O, O—(C₁-C₆ alkyl), NH₂, NHCH₃, or ═O;
k is 1 or 2;
m, u, and v are, independently, 0, 1, or 2;
n, p, q, r, s, and t are, independently, 0, 1, 2, 3, 4, 5, or 6; and,
the dashed line represents an optional double bond; or,
a pharmaceutically acceptable salt thereof.

2. The 5HT2 antagonist of claim 1, wherein R₁, R₂, and R₃ are, independently, hydrogen or methyl.

3. The 5HT2 antagonist of claim 1, wherein R₁, R₂, and R₃ are, independently, hydrogen or methyl; and, R₇ is O(CH₂)ᵣCH₃.

4. The 5HT2 antagonist of claim 2, wherein R₄ is hydrogen, fluoro, or methyl; and R₅ is CR₁₁R₁₂—(CH₂)ₙCH₃.

5. The 5HT2 antagonist of claim 3, wherein R₁ and R₃ are both hydrogen; and, R₂ is methyl.

6. The 5HT2 antagonist of claim 4, wherein R₁₁, and R₁₂ are, independently, H, OH, CO—NH—(C₁-C₆ alkyl), CO—NH-aryl, or O—(C₁-C₆ alkyl).

7. The 5HT2 antagonist of claim 6, wherein R₈ is fluoro.

8. The 5HT2 antagonist of claim 2, wherein R₇ is OCH₃.

9. The 5HT2 antagonist of claim 8, wherein R₁ and R₃ are both hydrogen, and R₂ is methyl.

10. The 5HT2 antagonist of claim 1, wherein R₄ is fluoro and R₅ is chloro.

11. The 5HT2 antagonist of claim 1, wherein said 5HT2 antagonist is a 5HT2A or 5HT2C antagonist.

12. The 5HT2 antagonist of claim 1, selected from the group consisting of:
[4-Chloro-5-fluoro-2-(3-methoxy-2-methylphenoxy)benzyl]methylamine, hydrochloride salt;
3-(5-Chloro-4-fluoro-2-methylaminomethylphenoxy)-2-methylphenol;
[4-Bromo-2-(3-methoxy-2-methylphenoxy)benzyl]methylamine;
2-[3-(3-Methoxy-2-methylphenoxy)-4-methylaminomethylphenyl]propan-2-ol;
[4-Chloro-2-(3-methoxy-2-methylphenoxy)-5-methylbenzyl]methylamine;
[2-(4-Chlorophenoxy)-4-methanesulfonylbenzyl]methylamine;
1-[3-(4-Chloro-3-methoxy-2-methylphenoxy)-4-methylaminomethylphenyl]propan-1-ol;
[4-Chloro-2-(1-methyl-1H-indol-4-yloxy)benzyl]methylamine;
[4-Chloro-2-(4-chloro-3-methoxy-2-methylphenoxy)-5-fluorobenzyl]methylamine;
[4-Bromo-2-(4-chloro-3-methoxy-2-methylphenoxy)benzyl]methylamine;
6-Chloro-3-(5-chloro-4-fluoro-2-methylaminomethylphenoxy)-2-methylphenol;
[4-Chloro-2-(3-methoxy-2-methylphenoxy)benzyl]methylamine;
[4-Bromo-2-(3-methoxy-2-methylphenoxy)-5-methylbenzyl]methylamine;
1-[3-(4-Chloro-2,3-dimethylphenoxy)-4-methylaminomethylphenyl]propan-1-ol;
[4-Chloro-2-(4-chloro-2,3-dimethylphenoxy)-5-fluorobenzyl]methylamine;
[2-(4-Chloro-2,3-dimethylphenoxy)-4-methanesulfonylbenzyl]methylamine;
[4-Chloro-2-(4-chloro-2,3-dimethylphenoxy)-5-methylbenzyl]methylamine;
[4-Bromo-2-(4-chloro-2,3-dimethylphenoxy)benzyl]methylamine;
[2-(4-Chloro-2,3-dimethylphenoxy)-4-methanesulfonyl-5-methylbenzyl]methylamine;
[2-(4-Chloro-2,3-dimethylphenoxy)-4-methanesulfinyl-5-methylbenzyl]methylamine;
[4-Chloro-2-(4-chloro-2,3-dimethylphenoxy)benzyl]methylamine;
1-[3-(4-Chloro-2,3-dimethylphenoxy)-4-methylaminomethylphenyl]ethanol;
[3-(4-Chloro-2,3-dimethylphenoxy)-4-methylaminomethylphenyl]methanol;
[2-(4-Chloro-2,3-dimethylphenoxy)-4-(pyrrolidine-1-sulfonyl)benzyl]methylamine;
[2-(4-Chloro-2,3-dimethylphenoxy)-4-methoxymethylbenzyl]methylamine;
[2-(4-Chloro-2,3-dimethylphenoxy)-4-(1-methoxypropyl)benzyl]methylamine;
[2-(4-Chloro-2,3-dimethylphenoxy)-4-(1-methoxyethyl)benzyl]methylamine;
[4-Chloro-2-(4-chloro-2,3-dimethylphenoxy)-5-fluorobenzyl]methylamine;
1-[3-(4-Chloro-2,3-dimethylphenoxy)-4-methylaminomethylphenyl]-prop-2-yn-1-ol;
1-[3-(4-Chloro-2,3-dimethylphenoxy)-4-methylaminomethylphenyl]-pent-4-en-1-ol;
1-[3-(4-Chloro-2,3-dimethylphenoxy)-4-methylaminomethylphenyl]-3-phenyl-prop-2-yn-1-ol; and,
[3-(4-Chloro-2,3-dimethylphenoxy)-4-methylaminomethylphenyl]-(2-methoxy-phenyl)-methanol.

13. The 5HT2 antagonist of claim 1, selected from the group consisting of:
(S)-1-[2-Fluoro-5-(7-fluoroindan-4-yloxy)-4-methylaminomethylphenyl]propan-1-ol;
1-[3-(2-Chloro-4-fluoro-3-methylphenoxy)-4-methylaminomethylphenyl]propan-1-ol;
1-[3-(2-Chloro-4-fluoro-3-methylphenoxy)-4-methylaminomethylphenyl]propan-1-ol;
1-[3-(4-Chloro-2,3-dimethylphenoxy)-4-methylaminomethylphenyl]-2-methylprop-2-en-1-ol;
1-[3-(4-Chloro-2,3-dimethylphenoxy)-4-methylaminomethylphenyl]-but-3-en-1-ol;
[3-(4-Chloro-2,3-dimethylphenoxy)-4-methylaminomethylphenyl]-(3-fluorophenyl)methanol;
1-[3-(3-Methoxy-2-methylphenoxy)-4-methylaminomethylphenyl]propan-1-ol;
[2-(3-Methoxy-2-methylphenoxy)-4-(1-methoxypropyl)benzyl]methylamine;
[2-(4-Chloro-3-methoxy-2-methylphenoxy)-4-(1-methoxypropyl)benzyl]methylamine;
[4-Chloro-2-(4-chloro-2-fluorophenoxy)benzyl]methylamine;
[4-Chloro-2-(4-chloro-2-fluorophenoxy)-5-fluorobenzyl]methylamine;
[4-Chloro-2-(4-chloro-2-fluorophenoxy)-5-fluorobenzyl]dimethylamine;
[2-(4-Chloro-2-fluorophenoxy)-5-fluoro-4-methylbenzyl]methylamine;
{1-[4-Chloro-2-(4-chloro-2-fluorophenoxy)-5-fluorophenyl]ethyl}methylamine;
{1-[4-Chloro-2-(4-chloro-2-fluorophenoxy)-5-fluorophenyl]ethyl}dimethylamine;
[4-Chloro-2-(4-chloro-2-fluorophenoxy)-5-methylbenzyl]methylamine;
[5-Chloro-2-(4-chloro-2-fluorophenoxy)-4-methylbenzyl]methylamine;
[2-(4-Chloro-2-fluorophenoxy)-4,5-dimethylbenzyl]methylamine;
[2-(4-Chloro-2-fluorophenoxy)-4-methoxybenzyl]methylamine;
[2-(4-Chloro-2-fluorophenoxy)-4-methylbenzyl]methylamine;
[2-(4-Chloro-2-fluorophenoxy)-5-fluoro-4-propylsulfanylbenzyl]methylamine;
[2-(4-Chloro-2-fluorophenoxy)-5-fluoro-4-isopropylsulfanylbenzyl]methylamine;
[4-Bromo-2-(4-chloro-2-fluorophenoxy)benzyl]methylamine;
[4-Chloro-2-(4-chloro-2-fluoro-3-methylphenoxy)-5-fluorobenzyl]methylamine;
[2-(4-Chloro-2-fluorophenoxy)-5-fluoro-4-methanesulfonylbenzyl]methylamine;
[4-(Butane-1-sulfonyl)-2-(4-chloro-2-fluorophenoxy)-5-fluorobenzyl]methylamine;
[2-(4-Chloro-2-fluorophenoxy)-5-fluoro-4-(propane-1-sulfonyl)benzyl]methylamine;
[2-(4-Chloro-2-fluoro-3-methylphenoxy)-4-methanesulfonylbenzyl]methylamine;
1-[3-(4-Chloro-2-fluorophenoxy)-4-methylaminomethylphenyl]propan-1-ol;
[4-Chloro-2-(1,3-dihydrobenzo[c]thiophen-5-yloxy)benzyl]methylamine;
[4-Chloro-2-(2,3-dihydrobenzo[b]thiophen-6-yloxy)benzyl]methylamine;
[4-Chloro-2-(2,3-dihydrobenzo[b]thiophen-5-yloxy)benzyl]methylamine;
[4-Chloro-2-(1,3-dihydroisobenzofuran-5-yloxy)benzyl]methylamine;
[4-Chloro-2-(7-fluoroindan-4-yloxy)benzyl]methylamine;
[2-(Indan-5-yloxy)-4-methanesulfonylbenzyl]methylamine;
[4-Methanesulfonyl-2-(naphthalen-2-yloxy)benzyl]methylamine;
[4-Chloro-2-(1-methyl-1,2,3,4-tetrahydroquinolin-6-yloxy)benzyl]methylamine;
[2-(4-Chlorophenoxy)-4-ethylsulfanylbenzyl]methylamine;
4-(5-Chloro-4-fluoro-2-methylaminomethylphenoxy)-2-methylphenol;
[4-Bromo-2-(1,4-dimethyl-1H-indol-5-yloxy)benzyl]methylamine;
[4-Chloro-2-(1,4-dimethyl-2,3-dihydro-1H-indol-5-yloxy)-5-fluorobenzyl]methylamine; and,
[4-Chloro-2-(1,4-dimethyl-1H-indol-5-yloxy)-5-methylbenzyl]methylamine.

14. A pharmaceutical composition for use in treating a disorder or condition in a mammal selected from depression, anxiety, depression with concomitant anxiety, dysthymia, post traumatic stress disorder, panic phobias, obsessive compulsive disorder (OCD), OCD with comorbid Tourette's Syndrome, borderline personality disorder, sleep disorder, psychosis, seizures, dyskinesis, symptoms of Huntington's or Parkinson's diseases, spasticity, suppression of seizures resulting from epilepsy, cerebral ischemia, anorexia, faintness attacks, hypokinesia, cranial traumas, chemical dependencies, premature ejaculation, premenstrual syndrome (PMS) associated mood and appetite disorder, inflammatory bowel disease, modification of feeding behavior, blocking carbohydrate cravings, late luteal phase dysphoric disorder, tobacco withdrawal-associated symptoms, panic disorder, bipolar disorder, sleep disorders, jet lag, cognitive dysfunction, hypertension, bulimia, anorexia, obesity, cardiac arrhythmias, chemical dependencies and addictions selected from dependencies on, or addictions to nicotine or tobacco products, alcohol, benzodiazepines, barbiturates, opioids or cocaine; pathological gambling; trichotilomania; headache, stroke, traumatic brain injury (TBI), psychosis, Huntington's Chorea, tardive dyskinesia, hyperkinesia, dyslexia, schizophrenia, multi-infarct dementia, epilepsy, senile dementia of the Alzheimer's type (AD), Parkinson's disease (PD), attention deficit hyperactivity disorder (ADHD) and Tourette's Syndrome, comprising an amount of the 5HT2 antagonist of claim 1, or a pharmaceutically acceptable salt thereof, that is effective in treating such disorder or condition and a pharmaceutically acceptable carrier.

15. A method of treating a disorder or condition in a mammal selected from depression, anxiety, depression with concomitant anxiety, dysthymia, post traumatic stress disorder, panic phobias, obsessive compulsive disorder (OCD), OCD with comorbid Tourette's Syndrome, borderline personality disorder, sleep disorder, psychosis, seizures, dyskinesis, symptoms of Huntington's or Parkinson's diseases, spasticity, suppression of seizures resulting from epilepsy, cerebral ischemia, anorexia, faintness attacks, hypokinesia, cranial traumas, chemical dependencies, premature ejaculation, premenstrual syndrome (PMS) associated mood and appetite disorder, inflammatory bowel disease, modification of feeding behavior, blocking carbohydrate cravings, late luteal phase dysphoric disorder, tobacco withdrawal-associated symptoms, panic disorder, bipolar disorder, sleep disorders, jet lag, cognitive dysfunction, hypertension, bulimia, anorexia, obesity, cardiac arrhythmias, chemical dependencies and addictions selected from dependencies on, or addictions to nicotine or tobacco products, alcohol, benzodiazepines, barbiturates, opioids or cocaine; pathological gambling; trichotilomania; headache, stroke, traumatic brain injury (TBI), psychosis, Huntington's Chorea, tardive dyskinesia, hyperkinesia, dyslexia, schizophrenia, multi-infarct dementia, epilepsy, senile dementia of the Alzheimer's type (AD), Parkinson's disease (PD), attention deficit hyperactivity disorder (ADHD) and Tourette's Syndrome, comprising administering to a mammal in need of such treatment an amount of the 5HT2 antagonist of claim 1, or a pharmaceutically acceptable salt thereof, that is effective in treating such disorder or condition.

16. The method of claim 15, wherein said disease or disorder is depression.

17. The method of claim 15, wherein the 5HT2 antagonist is selected from the group consisting of:
[4-Chloro-5-fluoro-2-(3-methoxy-2-methylphenoxy)benzyl]methylamine, hydrochloride salt;
3-(5-Chloro-4-fluoro-2-methylaminomethylphenoxy)-2-methylphenol;
[4-Bromo-2-(3-methoxy-2-methylphenoxy)benzyl]methylamine;
2-[3-(3-Methoxy-2-methylphenoxy)-4-methylaminomethylphenyl]propan-2-ol;
[4-Chloro-2-(3-methoxy-2-methylphenoxy)-5-methylbenzyl]methylamine;
[2-(4-Chlorophenoxy)-4-methanesulfonylbenzyl]methylamine;
1-[3-(4-Chloro-3-methoxy-2-methylphenoxy)-4-methylaminomethylphenyl]propan-1-ol;
[4-Chloro-2-(1-methyl-1H-indol-4-yloxy)benzyl]methylamine;
[4-Chloro-2-(4-chloro-3-methoxy-2-methylphenoxy)-5fluorobenzyl]methylamine;
[4-Bromo-2-(4-chloro-3-methoxy-2-methylphenoxy)benzyl]methylamine;
6-Chloro-3-(5-chloro-4-fluoro-2-methylaminomethylphenoxy)-2-methylphenol;
[4-Chloro-2-(3-methoxy-2-methylphenoxy)benzyl]methylamine;
[4-Bromo-2-(3-methoxy-2-methylphenoxy)-5-methylbenzyl]methylamine;
1-[3-(4-Chloro-2,3-dimethylphenoxy)-4-methylaminomethylphenyl]propan-1-ol;
[4-Chloro-2-(4-chloro-2,3-dimethylphenoxy)-5-fluorobenzyl]methylamine;
[2-(4-Chloro-2,3-dimethylphenoxy)-4-methanesulfonylbenzyl]methylamine;
[4-Chloro-2-(4-chloro-2,3-dimethylphenoxy)-5-methylbenzyl]methylamine;
[4-Bromo-2-(4-chloro-2,3-dimethylphenoxy)benzyl]methylamine;
[2-(4-Chloro-2,3-dimethylphenoxy)-4-methanesulfonyl-5-methylbenzyl]methylamine;
[2-(4-Chloro-2,3-dimethylphenoxy)-4-methanesulfinyl-5-methylbenzyl]methylamine;
[4-Chloro-2-(4-chloro-2,3-dimethylphenoxy)benzyl]methylamine;
1-[3-(4-Chloro-2,3-dimethylphenoxy)-4-methylaminomethylphenyl]ethanol;
[3-(4-Chloro-2,3-dimethylphenoxy)-4-methylaminomethylphenyl]methanol;
[2-(4-Chloro-2,3-dimethylphenoxy)-4-(pyrrolidine-1-sulfonyl)benzyl]methylamine;
[2-(4-Chloro-2,3-dimethylphenoxy)-4-methoxymethylbenzyl]methylamine;
[2-(4-Chloro-2,3-dimethylphenoxy)-4-(1-methoxypropyl)benzyl]methylamine;
[2-(4-Chloro-2,3-dimethylphenoxyy-4-(1-methoxyethyl)benzyl]methylamine;
[4-Chloro-2-(4-chloro-2,3-dimethylphenoxy)-5-fluorobenzyl]methylamine;
1-[3-(4-Chloro-2,3-dimethylphenoxy)-4-methylaminomethylphenyl]prop-2-yn-1-ol;
1-[3-(4-Chloro-2,3-dimethylphenoxy)-4-methylaminomethylphenyl]-pent-4-en-1-ol;
1-[3-(4-Chloro-2,3-dimethylphenoxy)-4-methylaminomethylphenyl]-3-phenyl-prop-2-yn-1-ol; and,
[3-(4-Chloro-2,3-dimethylphenoxy)-4-methylaminomethylphenyl]-(2-methoxy-phenyl)-methanol.

18. The method of claim 15, wherein the 5HT2 antagonist is selected from the group consisting of:
1-[3-(4-Chloro-2,3-dimethylphenoxy)-4-methylaminomethylphenyl]-2-methyl-prop-2-en-1-ol;
1-[3-(4-Chloro-2,3-dimethylphenoxy)-4-methylaminomethylphenyl]-but-3-en-1-ol;
[3-(4-Chloro-2,3-dimethylphenoxy)-4-methylaminomethylphenyl]-(3-fluorophenyl)-methanol;
1-[3-(3-Methoxy-2-methylphenoxy)-4-methylaminomethylphenyl]propan-1-ol;
[2-(3-Methoxy-2-methylphenoxy)-4-(1-methoxypropyl)benzyl]methylamine;
[2-(4-Chloro-3-methoxy-2-methylphenoxy)-4-(1-methoxypropyl)benzyl]methylamine;
[4-Chloro-2-(4-chloro-2-fluorophenoxy)benzyl]methylamine;
[4-Chloro-2-(4-chloro-2-fluorophenoxy)-5-fluorobenzyl]methylamine;
[4-Chloro-2-(4-chloro-2-fluorophenoxy)-5-fluorobenzyl]-dimethylamine;
[2-(4-Chloro-2-fluorophenoxy)-5-fluoro-4-methylbenzyl]methylamine;
{1-[4-Chloro-2-(4-chloro-2-fluorophenoxy)-5-fluorophenyl]ethyl}-methylamine;
{1-[4-Chloro-2-(4-chloro-2-fluorophenoxy)-5-fluorophenyl]ethyl}-dimethylamine;
[4-Chloro-2-(4-chloro-2-fluorophenoxy)-5-methylbenzyl]methylamine;
[5-Chloro-2-(4-chloro-2-fluorophenoxy)-4-methylbenzyl]methylamine;
[2-(4-Chloro-2-fluorophenoxy)-4,5-dimethylbenzyl]methylamine;
[2-(4-Chloro-2-fluorophenoxy)-4-methoxy-benzyl]methylamine;
[2-(4-Chloro-2-fluorophenoxy)-4-methylbenzyl]methylamine;
[2-(4-Chloro-2-fluorophenoxy)-5-fluoro-4-propylsulfanylbenzyl]methylamine;
[2-(4-Chloro-2-fluorophenoxy)-5-fluoro-4-isopropylsulfanylbenzyl]methylamine;
[4-Bromo-2-(4-chloro-2-fluorophenoxy)benzyl]methylamine;
[4-Chloro-2-(4-chloro-2-fluoro-3-methylphenoxy)-5-fluorobenzyl]methylamine;
[2-(4-Chloro-2-fluorophenoxy)-5-fluoro-4-methanesulfonylbenzyl]methylamine;
[4-(Butane-1-sulfonyl)-2-(4-chloro-2-fluorophenoxy)-5-fluorobenzyl]methylamine;
[2-(4-Chloro-2-fluorophenoxy)-5-fluoro-4-(propane-1-sulfonyl)benzyl]methylamine;
[2-(4-Chloro-2-fluoro-3-methylphenoxy)-4-methanesulfonylbenzyl]methylamine;
1-[3-(4-Chloro-2-fluorophenoxy)-4-methylaminomethylphenyl]propan-1-ol;
[4-Chloro-2-(1,3-dihydrobenzo[c]thiophen-5-yloxy)benzyl]methylamine;
[4-Chloro-2-(2,3-dihydrobenzo[b]thiophen-6-yloxy)benzyl]methylamine;
[4-Chloro-2-(2,3-dihydrobenzo[b]thiophen-5-yloxy)benzyl]methylamine;
[4-Chloro-2-(1,3-dihydro-isobenzofuran-5-yloxy)benzyl]methylamine;
[4-Chloro-2-(7-fluoroindan-4-yloxy)benzyl]methylamine;
[2-(Indan-5-yloxy)-4-methanesulfonylbenzyl]methylamine;
[4-Methanesulfonyl-2-(naphthalen-2-yloxy)benzyl]methylamine;
[4-Chloro-2-(1-methyl-1,2,3,4-tetrahydro-quinolin-6-yloxy)benzyl]methylamine;
[2-(4-Chlorophenoxy)-4-ethylsulfanylbenzyl]methylamine;
4-(5-Chloro-4-fluoro-2-methylaminomethylphenoxy)-2-methylphenol;
[4-Bromo-2-(1,4-dimethyl-1H-indol-5-yloxy)benzyl]methylamine;
[4-Chloro-2-(1,4-dimethyl-2,3-dihydro-1H-indol-5-yloxy)-5-fluorobenzyl]methylamine; and,
[4-Chloro-2-(1,4-dimethyl-1H-indol-5-yloxy)-5-methylbenzyl]methylamine.

19. A compound having the formula Ia, Ib or Ic: 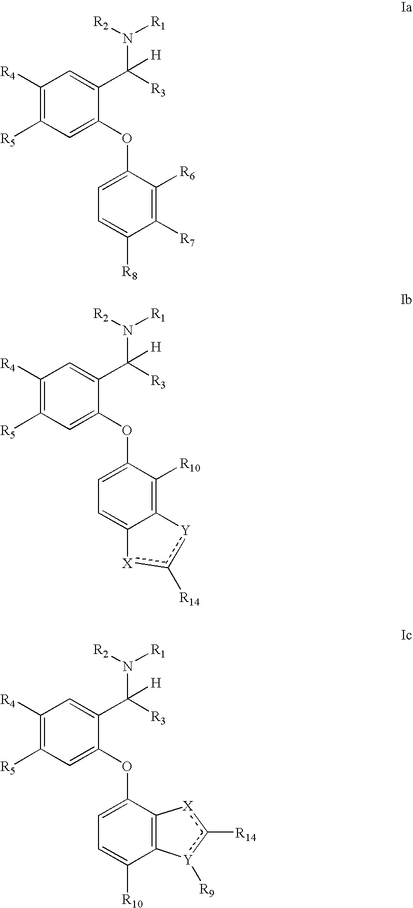
wherein X and Y are independently O, O(CH₂)ₙ, S, S(CH₂)ₙ, N, NR₁₈, NR₁₈N, NR₁₈(CH₂)ₙ, CR₁₈R₁₉(CH₂)ₙ or (CR₁₈R₁₉)ₖ, where R₁₈ and R₁₉ are independently H, straight chain or branched C₁-C₆ alkyl, CF₃, CN;
R₁, R₂ and R₃ are, independently, H or CH₃;
R₄ is H, F, Cl or CH₃; R₅ is F, Cl, Br, C₁-C₆ alkyl, (CH₂)ₙCN, (CH₂)ₙOH, (CH₂)ₙCO₂Et, C₁-C₆ cycloalkyl, oxazolyl or substituted oxazolyl, CR₁₁R₁₂—(CH₂)ₙCH₃, or S(O)ₘ—(CH₂)ₚCH₃;
R₆ is H, F, Cl, Br, O(CH₂)ᵣCH₃, C₁-C₆ alkyl, or CN;
R₇ is H, F, Cl, Br, C₁-C₆ alkyl, O—(CH₂)ₛCH₃, Cl, CN, N(R₁₃)(R₁₅), or OH;
R₈ is H, F, Cl, Br, C₁-C₆ alkyl, O—(C₁-C₆ alkyl), S—(C₁-C₆ alkyl), OH, NH—R₁₆, or S(O)ₜ—(C₁-C₆) alkyl; with the proviso that when R₄ is H, then only one of R₆, R₇ and R₈ may be H, and no two of R₆, R₇ and R₈ are the same;
R₉ is H, CH₃, OH, or, if Y is C, R₉ may alternatively be ═O;
R₁₀ is H, Cl, F, Br, C₁-C₆ alkyl, O—(C₁-C₆ alkyl), S—(C₁-C₆ alkyl), OH, NH—R₇, or S(O)ᵤ—(C₁-C₆ alkyl); or, R₆ and R₇, or R₇ and R₈, or R₉ and R₁₀, together with the atoms to which they are attached, form a 5- to 8-membered ring containing one or more heteroatoms selected from the group consisting of N, O, and S;
R₁₁ and R₁₂ are, independently, H, OH, O—(C₁-C₆ alkyl), C₁-C₆ alkyl, S(O)ᵥ—(C₁-C₆ alkyl), CO—NH—(C₁-C₆ alkyl), O—(C₁-C₆ alkyl), (CH₂)ₙ—S(O)ₘ—(C₁-C₆ alkyl), or CO—NH-aryl;
R₁₃, R₁₅, R₁₆, and R₁₇ are, independently, H, or C₁-C₆ alkyl;
R₁₄ is H, CH₃, Cl, OH, O, O—(C₁-C₆ alkyl), NH₂, NHCH₃, or ═O;
k is 1 or 2;
m, u, and v are, independently, 0, 1, or 2;
n, p, q, r, s, and t are, independently, 0, 1, 2, 3, 4, 5, or 6; and,
the dashed line represents an optional double bond; or,
a pharmaceutically acceptable salt thereof.

20. The compound of claim 19, wherein R₁, R₂, and R₃ are, independently, hydrogen or methyl.

21. The compound of claim 19, wherein said 5HT2 antagonist is a 5HT2A or 5HT2C antagonist.

22. The compound of claim 19 selected from the group consisting of:
[4-Chloro-5-fluoro-2-(3-methoxy-2-methylphenoxy)benzyl]methylamine, hydrochloride salt;
3-(5-Chloro-4-fluoro-2-methylaminomethylphenoxy)-2-methylphenol;
[4-Bromo-2-(3-methoxy-2-methylphenoxy)benzyl]methylamine;
2-[3-(3-Methoxy-2-methylphenoxy)-4-methylaminomethylphenyl]propan-2-ol;
[4-Chloro-2-(3-methoxy-2-methylphenoxy)-5-methylbenzyl]methylamine;
[2-(4-Chlorophenoxy)-4-methanesulfonylbenzyl]methylamine;
1-[3-(4-Chloro-3-methoxy-2-methylphenoxy)-4-methylaminomethylphenyl]propan-1-ol;
[4-Chloro-2-(1-methyl-1H-indol-4-yloxy)benzyl]methylamine;
[4-Chloro-2-(4-chloro-3-methoxy-2-methylphenoxy)-5-fluorobenzyl]methylamine;
[4-Bromo-2-(4-chloro-3-methoxy-2-methylphenoxy)benzyl]methylamine;
6-Chloro-3-(5-chloro-4-fluoro-2-methylaminomethylphenoxy)-2-methylphenol;
[4-Chloro-2-(3-methoxy-2-methylphenoxy)benzyl]methylamine;
[4-Bromo-2-(3-methoxy-2-methylphenoxy)-5-methylbenzyl]methylamine;
1-[3-(4-Chloro-2,3-dimethylphenoxy)-4-methylaminomethylphenyl]propan-1-ol;
[4-Chloro-2-(4-chloro-2,3-dimethylphenoxy)-5-fluorobenzyl]methylamine;
[2-(4-Chloro-2,3-dimethylphenoxy)-4-methanesulfonylbenzyl]methylamine;
[4-Chloro-2-(4-chloro-2,3-dimethylphenoxy)-5-methylbenzyl]methylamine;
[4-Bromo-2-(4-chloro-2,3-dimethylphenoxy)benzyl]methylamine;
[2-(4-Chloro-2,3-dimethylphenoxy)-4-methanesulfonyl-5-methylbenzyl]methylamine;
[2-(4-Chloro-2,3-dimethylphenoxy)-4-methanesulfinyl-5-methylbenzyl]methylamine;
[4-Chloro-2-(4-chloro-2,3-dimethylphenoxy)benzyl]methylamine;
1-[3-(4-Chloro-2,3-dimethylphenoxy)-4-methylaminomethylphenyl]ethanol;
[3-(4-Chloro-2,3-dimethylphenoxy)-4-methylaminomethylphenyl]methanol;
[2-(4-Chloro-2,3-dimethylphenoxy)-4-(pyrrolidine-1-sulfonyl)benzyl]methylamine;
[2-(4-Chloro-2,3-dimethylphenoxy)-4-methoxymethylbenzyl]methylamine;
[2-(4-Chloro-2,3-dimethylphenoxy)-4-(1-methoxypropyl)benzyl]methylamine;
[2-(4-Chloro-2,3-dimethylphenoxy)-4-(1-methoxyethyl)benzyl]methylamine;
[4-Chloro-2-(4-chloro-2,3-dimethylphenoxy)-5-fluorobenzyl]methylamine;
1-[3-(4-Chloro-2,3-dimethylphenoxy)-4-methylaminomethylphenyl]prop-2-yn-1-ol;
1-[3-(4-Chloro-2,3-dimethylphenoxy)-4-methylaminomethylphenyl]-pent-4-en-1-ol;
1-[3-(4-Chloro-2,3-dimethylphenoxy)-4-methylaminomethylphenyl]-3-phenyl-prop-2-yn-1-ol; and,
[3-(4-Chloro-2,3-dimethylphenoxy)-4-methylaminomethylphenyl]-(2-methoxy-phenyl)-methanol.

23. The compound of claim 19 selected from the group consisting of:
(S)-1-[2-Fluoro-5-(7-fluoroindan-4-yloxy)-4-methylaminomethylphenyl]propan-1-ol;
1-[3-(2-Chloro-4-fluoro-3-methylphenoxy)-4-methylaminomethylphenyl]propan-1-ol;
1-[3-(2-Chloro-4-fluoro-3-methylphenoxy)-4-methylaminomethylphenyl]propan-1-ol;
1-[3-(4-Chloro-2,3-dimethylphenoxy)-4-methylaminomethylphenyl]-2-methy-prop-2-en-1-ol;
1-[3-(4-Chloro-2,3-dimethylphenoxy)-4-methylaminomethylphenyl]-but-3-en-1-ol;
[3-(4-Chloro-2,3-dimethylphenoxy)-4-methylaminomethylphenyl]-(3-fluorophenyl)-methanol;
1-[3-(3-Methoxy-2-methylphenoxy)-4-methylaminomethylphenyl]propan-1-ol;
[2-(3-Methoxy-2-methylphenoxy)-4-(1-methoxypropyl)benzyl]methylamine;
[2-(4-Chloro-3-methoxy-2-methylphenoxy)-4-(1-methoxypropyl)benzyl]methylamine;
[4-Chloro-2-(4-chloro-2-fluorophenoxy)benzyl]methylamine;
[4-Chloro-2-(4-chloro-2-fluorophenoxy)-5-fluorobenzyl]methylamine;
[4-Chloro-2-(4-chloro-2-fluorophenoxy)-5-fluorobenzyl]-dimethylamine;
[2-(4-Chloro-2-fluorophenoxy)-5-fluoro-4-methylbenzyl]methylamine;
{1-[4-Chloro-2-(4-chloro-2-fluorophenoxy)-5-fluorophenyl]ethyl}-methylamine;
{1-[4-Chloro-2-(4-chloro-2-fluorophenoxy)-5-fluorophenyl]ethyl}-dimethylamine;
[4-Chloro-2-(4-chloro-2-fluorophenoxy)-5-methylbenzyl]methylamine;
[5-Chloro-2-(4-chloro-2-fluorophenoxy)-4-methylbenzyl]methylamine;
[2-(4-Chloro-2-fluorophenoxy)-4,5-dimethylbenzyl]methylamine;
[2-(4-Chloro-2-fluorophenoxy)-4-methoxy-benzyl]methylamine;
[2-(4-Chloro-2-fluorophenoxy)-4-methylbenzyl]methylamine;
[2-(4-Chloro-2-fluorophenoxy)-5-fluoro-4-propylsulfanylbenzyl]methylamine;
[2-(4-Chloro-2-fluorophenoxy)-5-fluoro-4-isopropylsulfanylbenzyl]methylamine;
[4-Bromo-2-(4-chloro-2-fluorophenoxy)benzyl]methylamine;
[4-Chloro-2-(4-chloro-2-fluoro-3-methylphenoxy)-5-fluorobenzyl]methylamine;
[2-(4-Chloro-2-fluorophenoxy)-5-fluoro-4-methanesulfonylbenzyl]methylamine;
[4-(Butane-1-sulfonyl)-2-(4-chloro-2-fluorophenoxy)-5-fluorobenzyl]methylamine;
[2-(4-Chloro-2-fluorophenoxy)-5-fluoro-4-(propane-1-sulfonyl)benzyl]methylamine;
[2-(4-Chloro-2-fluoro-3-methylphenoxy)-4-methanesulfonylbenzyl]methylamine;
1-[3-(4-Chloro-2-fluorophenoxy)-4-methylaminomethylphenyl]propan-1-ol;
[4-Chloro-2-(1,3-dihydrobenzo[c]thiophen-5-yloxy)benzyl]methylamine;
[4-Chloro-2-(2,3-dihydrobenzo[b]thiophen-6-yloxy)benzyl]methylamine;
[4-Chloro-2-(2,3-dihydrobenzo[b]thiophen-5-yloxy)benzyl]methylamine;
[4-Chloro-2-(1,3-dihydro-isobenzofuran-5-yloxy)benzyl]methylamine;
[4-Chloro-2-(7-fluoroindan-4-yloxy)benzyl]methylamine;
[2-(Indan-5-yloxy)-4-methanesulfonylbenzyl]methylamine;
[4-Methanesulfonyl-2-(naphthalen-2-yloxy)benzyl]methylamine;
[4-Chloro-2-(1-methyl-1,2,3,4-tetrahydro-quinolin-6-yloxy)benzyl]methylamine;
[2-(4-Chlorophenoxy)-4-ethylsulfanylbenzyl]methylamine;
4-(5-Chloro-4-fluoro-2-methylaminomethylphenoxy)-2-methylphenol;
[4-Bromo-2-(1,4-dimethyl-1H-indol-5-yloxy)benzyl]methylamine;
[4-Chloro-2-(1,4-dimethyl-2,3-dihydro-1H-indol-5-yloxy)-5-fluorobenzyl]methylamine; and,
[4-Chloro-2-(1,4-dimethyl-1H-indol-5-yloxy)-5-methylbenzyl]methylamine.

24. A pharmaceutical composition for use in treating a disorder or condition in a mammal selected from depression, anxiety, depression with concomitant anxiety, dysthymia, post traumatic stress disorder, panic phobias, obsessive compulsive disorder (OCD), OCD with comorbid Tourette's Syndrome, borderline personality disorder, sleep disorder, psychosis, seizures, dyskinesis, symptoms of Huntington's or Parkinson's diseases, spasticity, suppression of seizures resulting from epilepsy, cerebral ischemia, anorexia, faintness affacks, hypokinesia, cranial traumas, chemical dependencies, premature ejaculation, premenstrual syndrome (PMS) associated mood and appetite disorder, inflammatory bowel disease, modification of feeding behavior, blocking carbohydrate cravings, late luteal phase dysphoric disorder, tobacco withdrawal-associated symptoms, panic disorder, bipolar disorder, sleep disorders, jet lag, cognitive dysfunction, hypertension, bulimia, anorexia, obesity, cardiac arrhythmias, chemical dependencies and addictions selected from dependencies on, or addictions to nicotine or tobacco products, alcohol, benzodiazepines, barbiturates, opioids or cocaine; pathological gambling; trichotilomania; headache, stroke, traumatic brain injury (TBI), psychosis, Huntington's Chorea, tardive dyskinesia, hyperkinesia, dyslexia, schizophrenia, multi-infarct dementia, epilepsy, senile dementia of the Alzheimer's type (AD), Parkinson's disease (PD), attention deficit hyperactivity disorder (ADHD) and Tourette's Syndrome, comprising an amount of the compound of claim 19, or a pharmaceutically acceptable salt thereof, that is effective in treating such disorder or condition and a pharmaceutically acceptable carrier.

25. A method of treating a disorder or condition in a mammal selected from depression, anxiety, depression with concomitant anxiety, dysthymia, post traumatic stress disorder, panic phobias, obsessive compulsive disorder (OCD), OCD with comorbid Tourette's Syndrome, borderline personality disorder, sleep disorder, psychosis, seizures, dyskinesis, symptoms of Huntington's or Parkinson's diseases, spasticity, suppression of seizures resulting from epilepsy, cerebral ischemia, anorexia, faintness attacks, hypokinesia, cranial traumas, chemical dependencies, premature ejaculation, premenstrual syndrome (PMS) associated mood and appetite disorder, inflammatory bowel disease, modification of feeding behavior, blocking carbohydrate cravings, late luteal phase dysphoric disorder, tobacco withdrawal-associated symptoms, panic disorder, bipolar disorder, sleep disorders, jet lag, cognitive dysfunction, hypertension, bulimia, anorexia, obesity, cardiac arrhythmias, chemical dependencies and addictions selected from dependencies on, or addictions to nicotine or tobacco products, alcohol, benzodiazepines, barbiturates, opioids or cocaine; pathological gambling; trichotilomania; headache, stroke, traumatic brain injury (TBI), psychosis, Huntington's Chorea, tardive dyskinesia, hyperkinesia, dyslexia, schizophrenia, multi-infarct dementia, epilepsy, senile dementia of the Alzheimer's type (AD), Parkinson's disease (PD), attention deficit hyperactivity disorder (ADHD) and Tourette's Syndrome, comprising administering to a mammal in need of such treatment an amount of the compound of claim 19, or a pharmaceutically acceptable salt thereof, that is effective in treating such disorder or condition.

26. The method of claim 25 wherein said disease or disorder is depression.

27. The method of claim 25 wherein the compound is selected from the group consisting of:
[4-Chloro-5-fluoro-2-(3-methoxy-2-methylphenoxy)benzyl]methylamine, hydrochloride salt;
3-(5-Chloro-4-fluoro-2-methylaminomethylphenoxy)-2-methylphenol;
[4-Bromo-2-(3-methoxy-2-methylphenoxy)benzyl]methylamine;
2-[3-(3-Methoxy-2-methylphenoxy)-4-methylaminomethylphenyl]propan-2-ol;
[4-Chloro-2-(3-methoxy-2-methylphenoxy)-5-methylbenzyl]methylamine;
[2-(4-Chlorophenoxy)-4-methanesulfonylbenzyl]methylamine;
1-[3-(4-Chloro-3-methoxy-2-methylphenoxy)-4-methylaminomethylphenyl]propan-1-ol;
[4-Chloro-2-(1-methyl-1H-indol-4-yloxy)benzyl]methylamine;
[4-Chloro-2-(4-chloro-3-methoxy-2-methylphenoxy)-5-fluorobenzyl]methylamine;
[4-Bromo-2-(4-chloro-3-methoxy-2-methylphenoxy)benzyl]methylamine;
6-Chloro-3-(5-chloro-4-fluoro-2-methylaminomethylphenoxy)-2-methylphenol;
[4-Chloro-2-(3-methoxy-2-methylphenoxy)benzyl]methylamine;
[4-Bromo-2-(3-methoxy-2-methylphenoxy)-5-methylbenzyl]methylamine;
1-[3-(4-Chloro-2,3-dimethylphenoxy)-4-methylaminomethylphenyl]propan-1-ol;
[4-Chloro-2-(4-chloro-2,3-dimethylphenoxy)-5-fluorobenzyl]methylamine;
[2-(4-Chloro-2,3-dimethylphenoxy)-4-methanesulfonylbenzyl]methylamine;
[4-Chloro-2-(4-chloro-2,3-dimethylphenoxy)-5-methylbenzyl]methylamine;
[4-Bromo-2-(4-chloro-2,3-dimethylphenoxy)benzyl]methylamine;
[2-(4-Chloro-2,3-dimethylphenoxy)-4-methanesulfonyl-5-methylbenzyl]methylamine;
[2-(4-Chloro-2,3-dimethylphenoxy)-4-methanesulfinyl-5-methylbenzyl]methylamine;
[4-Chloro-2-(4-chloro-2,3-dimethylphenoxy)benzyl]methylamine;
1-[3-(4-Chloro-2,3-dimethylphenoxy)-4-methylaminomethylphenyl]ethanol;
[3-(4-Chloro-2,3-dimethylphenoxy)-4-methylaminomethylphenyl]methanol;
[2-(4-Chloro-2,3-dimethylphenoxy)-4-(pyrrolidine-1-sulfonyl)benzyl]methylamine;
[2-(4-Chloro-2,3-dimethylphenoxy)-4-methoxymethylbenzyl]methylamine;,
[2-(4-Chloro-2,3-dimethylphenoxy)-4-(1-methoxypropyl)benzyl]methylamine;
[2-(4-Chloro-2,3-dimethylphenoxy)-4-(1-methoxyethyl)benzyl]methylamine;
[4-Chloro-2-(4-chloro-2,3-dimethylphenoxy)-5-fluorobenzyl]methylamine;
1-[3-(4-Chloro-2,3-dimethylphenoxy)-4-methylaminomethylphenyl]prop-2-yn-1-ol;
1-[3-(4-Chloro-2,3-dimethylphenoxy)-4-methylaminomethylphenyl]pent-4-en-1-ol;
1-[3-(4-Chloro-2,3-dimethylphenoxy)-4-methylaminomethylphenyl]-3-phenylprop-2-yn-1-ol; and,
[3-(4-Chloro-2,3-dimethylphenoxy)-4-methylaminomethylphenyl]-(2-methoxyphenyl)-methanol.

28. The method of claim 25 wherein the compound is selected from the group consisting of:
(S)-1-[2-Fluoro-5-(7-fluoroindan-4-yloxy)-4-methylaminomethylphenyl]propan-1-ol;
1-[3-(2-Chloro-4-fluoro-3-methylphenoxy)-4-methylaminomethylphenyl]propan-1-ol;
1-[3-(2-Chloro-4-fluoro-3-methylphenoxy)-4-methylaminomethylphenyl]propan-1-ol;
1-[3-(4-Chloro-2,3-dimethylphenoxy)-4-methylaminomethylphenyl]-2-methylprop-2-en-1-ol;
1-[3-(4-Chloro-2,3-dimethylphenoxy)-4-methylaminomethylphenyl]-but-3-en-1-ol;
[3-(4-Chloro-2,3-dimethylphenoxy)-4-methylaminomethylphenyl]-(3-fluorophenyl)methanol;
1-[3-(3-Methoxy-2-methylphenoxy)-4-methylaminomethylphenyl]propan-1-ol;
[2-(3-Methoxy-2-methylphenoxy)-4-(1-methoxypropyl)benzyl]methylamine;
[2-(4-Chloro-3-methoxy-2-methylphenoxy)-4-(1-methoxypropyl)benzyl]methylamine;
[4-Chloro-2-(4-chloro-2-fluorophenoxy)benzyl]methylamine;
[4-Chloro-2-(4-chloro-2-fluorophenoxy)-5-fluorobenzyl]methylamine;
[4-Chloro-2-(4-chloro-2-fluorophenoxy)-5-fluorobenzyl]dimethylamine;
[2-(4-Chloro-2-fluorophenoxy)-5-fluoro-4-methylbenzyl]methylamine;
{1-[4-Chloro-2-(4-chloro-2-fluorophenoxy)-5-fluorophenyl]ethyl}methylamine;
{1-[4-Chloro-2-(4-chloro-2-fluorophenoxy)-5-fluorophenyl]ethyl}dimethylamine;
[4-Chloro-2-(4-chloro-2-fluorophenoxy)-5-methylbenzyl]methylamine;
[5-Chloro-2-(4-chloro-2-fluorophenoxy)-4-methylbenzyl]methylamine;
[2-(4-Chloro-2-fluorophenoxy)-4,5-dimethylbenzyl]methylamine;
[2-(4-Chloro-2-fluorophenoxy)-4-methoxybenzyl]methylamine;
[2-(4-Chloro-2-fluorophenoxy)-4-methylbenzyl]methylamine;
[2-(4-Chloro-2-fluorophenoxy)-5-fluoro-4-propylsulfanylbenzyl]methylamine;
[2-(4-Chloro-2-fluorophenoxy)-5-fluoro-4-isopropylsulfanylbenzyl]methylamine;
[4-Bromo-2-(4-chloro-2-fluorophenoxy)benzyl]methylamine;
[4-Chloro-2-(4-chloro-2-fluoro-3-methylphenoxy)-5-fluorobenzyl]methylamine;
[2-(4-Chloro-2-fluorophenoxy)-5-fluoro-4-methanesulfonylbenzyl]methylamine;
[4-(Butane-1-sulfonyl)-2-(4-chloro-2-fluorophenoxy)-5-fluorobenzyl]methylamine;
[2-(4-Chloro-2-fluorophenoxy)-5-fluoro-4-(propane-1-sulfonyl)benzyl]methylamine;
[2-(4-Chloro-2-fluoro-3-methylphenoxy)-4-methanesulfonylbenzyl]methylamine;
1-[3-(4-Chloro-2-fluorophenoxy)-4-methylaminomethylphenyl]propan-1-ol;
[4-Chloro-2-(1,3-dihydrobenzo[c]thiophen-5-yloxy)benzyl]methylamine;
[4-Chloro-2-(2,3-dihydrobenzo[b]thiophen-6-yloxy)benzyl]methylamine;
[4-Chloro-2-(2,3-dihydrobenzo[b]thiophen-5-yloxy)benzyl]methylamine;
[4-Chloro-2-(1,3-dihydroisobenzofuran-5-yloxy)benzyl]methylamine;
[4-Chloro-2-(7-fluoroindan-4-yloxy)benzyl]methylamine;
[2-(Indan-5-yloxy)-4-methanesulfonylbenzyl]methylamine;
[4-Methanesulfonyl-2-(naphthalen-2-yloxy)benzyl]methylamine;
[4-Chloro-2-(1-methyl-1,2,3,4-tetrahydroquinolin-6-yloxy)benzyl]methylamine;
[2-(4-Chlorophenoxy)-4-ethylsulfanylbenzyl]methylamine;
4-(5-Chloro-4-fluoro-2-methylaminomethylphenoxy)-2-methylphenol;
[4-Bromo-2-(1,4-dimethyl-1H-indol-5-yloxy)benzyl]methylamine;
[4-Chloro-2-(1,4-dimethyl-2,3-dihydro-1H-indol-5-yloxy)-5-fluorobenzyl]methylamine; and,
[4-Chloro-2-(1,4-dimethyl-1H-indol-5-yloxy)-5-methylbenzyl]methylamine.

29. A pharmaceutical composition for treating a condition or disorder that can be treated by enhancing serotonergic neurotransmission in a mammal, preferably a human, including:
a) a pharmaceutically acceptable carrier;
b) a compound of the formula Ia, Ib, or Ic, according to claim 1, or a pharmaceutically acceptable salt thereof; and,
c) a 5-HT re-uptake inhibitor, preferably sertraline, or a pharmaceutically acceptable salt thereof, or a norepinephrine reuptake inhibitor or pharmaceutically acceptable salt thereof, wherein the norepinephrine reuptake inhibitor is selected from the group consisting of racemic reboxetine, [S,S]-reboxetine, amoxapine, and maprotiline, wherein the amounts of the compound of formula Ia, Ib, or Ic and the 5-HT re-uptake inhibitor are such that the combination is effective in treating such disorder or condition.

30. A method for treating a disorder or condition that can be treated by enhancing serotonergic neurotransmission in a mammal, preferably a human, including administering to a mammal requiring such treatment:
a) a compound of the formula Ia, Ib, or Ic, according to claim 1, or a pharmaceutically acceptable salt thereof; and,
b) a 5-HT re-uptake inhibitor, preferably sertraline, or a pharmaceutically acceptable salt thereof, or a norepinephrine reuptake inhibitor or pharmaceutically acceptable salt thereof, wherein the norepinephrine reuptake inhibitor is selected from the group consisting of racemic reboxetine, [S,S]-reboxetine, amoxapine, and maprotiline, wherein the amounts of the compound of formula Ia, Ib, or Ic, the 5-HT re-uptake inhibitor and the norepinephrine reuptake inhibitor are such that the combination is effective in treating such disorder or condition.

31. A method for treating a disorder or condition that can be treated by enhancing serotonergic neurotransmission in a mammal, preferably a human, including administering to the mammal requiring such treatment:
a) a 5-HT_{1A} antagonist or a pharmaceutically acceptable salt thereof; and
b) a compound of formula Ia, Ib, or Ic, according to claim 1, or a pharmaceutically acceptable salt thereof, wherein the amounts of the 5-HT_{1A} antagonist and the compound of formula Ia, Ib, or Ic are such that the combination is effective in treating such disorder or condition.

32. A pharmaceutical composition for treating a disorder or condition that can be treated by enhancing serotonergic neurotransmission in a mammal, preferably a human, including:
a) a 5-HT_{1A} antagonist or a pharmaceutically acceptable salt thereof; and
b) a compound of formula Ia, Ib, or Ic, according to claim 1, or a pharmaceutically acceptable salt thereof, wherein the amounts of the 5-HT_{1A} antagonist and the compound of formula Ia, Ib, or Ic are such that the combination is effective in treating such disorder or condition.
